(19) United States
(12) Patent Application Publication
(10) Pub. No.: US 2024/0339190 A1
(43) Pub. Date: Oct. 10, 2024

(54) MANAGEMENT PROCESS FOR INSULIN THERAPY USING REINFORCEMENT LEARNING AND THERAPY ESCALATION PATHWAYS

(71) Applicant: Abbott Diabetes Care Inc., Alameda, CA (US)

(72) Inventors: Byron Paul OLSON, II, Sioux Falls, SD (US); Jeanne M. JACOBY, Belmont, MA (US); Matthew CLEMENTE, Milpitas, CA (US)

(21) Appl. No.: 18/629,078

(22) Filed: Apr. 8, 2024

Related U.S. Application Data
(60) Provisional application No. 63/494,865, filed on Apr. 7, 2023.
(60) Provisional application No. 63/560,371, filed on Mar. 1, 2024.

(51) Int. Cl. G16H 20/17; A61B 5/00; A61B 5/145

(52) U.S. Cl. CPC G16H 20/17; A61B 5/14532; A61B 5/4839

(57) ABSTRACT

A method of therapy escalation for patients with diabetes includes receiving glucose data of a user from an in vivo glucose monitoring device, receiving first therapy information of a first therapy, wherein the first therapy includes basal insulin, calculating one or more glucose metrics based on the received glucose data, titrating a dose of the basal insulin based on the one or more glucose metrics, and determining overbasalization based on one or more of the glucose data and the first therapy information. Advantageously the system can regularly monitor glucose control of a user, detect overbasalization, provide frequent therapy intervention and adjustment, decrease a duration of intervention, and increase user adherence, outcomes, and satisfaction.

### CROSS-REFERENCE TO RELATED APPLICATIONS

This application claims the benefit of U.S. Provisional Application No. 63/494,865, filed Apr. 7, 2023, and also claims the benefit of U.S. Provisional Application No. 63/560,371, filed Mar. 1, 2024, each of which applications are incorporated herein by reference in their entireties.

### FIELD

The present disclosure relates to therapy management apparatuses, systems, and methods, for example, a management process for insulin therapy using reinforcement learning and software application apparatuses, systems, and methods to detect the effectiveness of current therapy, detect overbasalization, and escalate to one or more therapy pathways.

### BACKGROUND

The detection and/or monitoring of glucose levels can be vitally important to the health of an individual having diabetes. People with diabetes (PWD) are generally required to monitor their glucose levels to ensure they are maintained within a clinically safe range, and may also use this information to determine if and/or when insulin is needed to reduce glucose levels in their bodies or when additional glucose is needed to raise glucose levels in their bodies. A number of systems allow individuals to monitor their glucose, for example, a continuous glucose monitor (CGM) to measure interstitial fluid glucose levels. Some of these systems include electrochemical biosensors, including those that use a glucose sensor adapted to be positioned in vivo, for example, with complete or partial insertion into a subcutaneous or transcutaneous site, within the body for continuous in vivo monitoring of glucose levels from bodily fluids of the site (e.g., interstitial fluid).

Diabetes mellitus is a chronic metabolic disorder caused by the inability of a person's pancreas to produce sufficient amounts of the hormone insulin, such that the metabolism is unable to provide for the proper absorption of sugar and starch. This failure can lead to hyperglycemia, e.g., the presence of an excessive amount of glucose. Persistent hyperglycemia has been associated with a variety of serious symptoms and long-term, life-threatening complications, such as dehydration, ketoacidosis, diabetic coma, cardiovascular diseases, chronic renal failure, retinal damage, and nerve damages with the risk of amputation.

A permanent therapy that provides constant glycemic control is needed in order to constantly maintain the level of glucose within acceptable limits. Such glycemic control can be achieved by regularly supplying external drugs to the body of PWD to thereby reduce the elevated levels of glucose. An external biologically effective drug (e.g., insulin or its analog) is commonly administered by means of daily injections. In some cases, multiple daily injections of a mixture of rapid-acting (RA) insulin and long-acting (LA) insulin are administered via a reusable transdermal liquid dosing device.

Glycated hemoglobin HbA1c (A1C) is a form of hemoglobin that is chemically linked to a sugar. The formation of the sugar-hemoglobin linkage can indicate the presence of excessive sugar in the bloodstream, often indicative of diabetes in high concentration. A1C is measured primarily to determine a 3-month average blood sugar level, and can be used as a diagnostic test for diabetes mellitus and as an assessment test for glycemic control in PWD. Generally, a normal A1C level is below 5.7%, a level of 5.7% to 6.4% indicates pre-diabetes, and a level of 6.5% or more indicates diabetes. Within the 5.7% to 6.4% pre-diabetes range, the higher the A1C level for PWD, the greater the risk is for developing type-2 diabetes.

Currently, methods to monitor A1C levels are limited to every 3-6 months depending on a variety of factors. This method of measuring patient progress and follow-up limited to quarterly or semi-annual visits can contribute to marked therapeutic inertia throughout the care of PWD, resulting in suboptimal outcomes and a “treat-to-failure” approach to diabetes therapy. Further, limited human resources continue to perpetuate the delay in advancing therapy according to standards of care.

### SUMMARY

Accordingly, there is a need to develop a therapy management system that can regularly monitor glucose control of a user, provide frequent therapy intervention and adjustment (e.g., on a daily or weekly basis rather than every 3-6 months), decrease a duration of intervention, and increase user adherence, outcomes, and satisfaction.

In an aspect, a non-transitory computer-readable storage medium has stored thereon instructions that when executed by a processor causes the processor to perform operations of a management process for insulin therapy, the operations comprising: performing a survey of a person with diabetes (PWD) and receiving survey information in response to the survey; performing a high-touch interaction phase with regard to the PWD, the high-touch interaction phase including repeated adjustments of the insulin therapy based on collected data and a publicly available guideline regarding the insulin therapy, the high-touch interaction phase establishing, for the PWD, at least a basal dose setting, a meal dose setting, and a correction dose setting; and performing, after termination of the high-touch interaction phase, an ongoing phase with regard to the PWD, the ongoing phase including collecting performance metrics for the PWD, applying reinforcement learning to the performance metrics, and automatically performing an action with regard to the PWD based on the reinforcement learning.

Implementations can include any or all of the following features. The high-touch interaction phase is performed for a predetermined length of time. The high-touch interaction phase has a goal-based duration. The goal-based duration depends on a determination that a blood glucose level of the PWD is within a target range, and that the PWD is taking insulin as specified in the insulin therapy. The operations further comprise evaluating each of multiple insights and determining whether the insight is triggered in view of at least the performance metrics and the collected data, wherein the action is selected based on at least one of the insights being triggered. The insights are evaluated according to priority with regard to the PWD. The insights being evaluated are selected from a set of insights based on having highest priority. Evaluating at least one of the insights comprises a multimodal evaluation. The operations further comprise performing observation with regard to a state of the PWD after automatically performing the action. The reinforcement learning comprises providing positive feedback or negative feedback to a selection of the action. The operations further comprise performing a cooldown after termination of the observation. The operations further comprise evaluating each of multiple insights and determining whether the insight is triggered in view of at least the performance metrics and the collected data, wherein the action is selected based on at least one of the insights being triggered. The operations further comprise checking, before selecting the action, whether the cooldown involves the action. If the cooldown involves the action, the operations instead evaluate a next insight of the multiple insights. The operations further comprise evaluating each of multiple insights and determining whether the insight is triggered in view of at least the performance metrics and the collected data, wherein the action is selected based on at least one of the insights being triggered. The operations further comprise checking, before selecting the action, whether the observation involves the insight. If the observation involves the insight, the operations evaluate an improvement criterion regarding the insight in view of at least the performance metrics and the collected data. Evaluating the improvement criterion comprises a multimodal evaluation. For any of the insights not being triggered in the evaluation, the operations further comprise determining whether that insight has not been triggered for at least a predetermined time, and performing a streak action in view of the determination. Automatically performing the action comprises selecting one or more goals for the PWD, and presenting the one or more goals to the PWD. The operations further comprise receiving an opt-in input or an opt-out input by the PWD.

In some aspects, a method of therapy escalation for PWD can include receiving glucose data of a user from an in vivo glucose monitoring device. In some aspects, the method can further include receiving first therapy information of a first therapy. In some aspects, the first therapy can include basal insulin. In some aspects, the method can further include calculating one or more glucose metrics based on the received glucose data. In some aspects, the method can further include titrating a dose of the basal insulin based on the one or more glucose metrics. In some aspects, the method can further include determining overbasalization based on one or more of the glucose data and the first therapy information.

In some aspects, the method can further include outputting a recommendation to add a second therapy when overbasalization is determined.

In some aspects, outputting the recommendation can include displaying the recommendation. In some aspects, displaying the recommendation can include displaying the recommendation on a display device, a remote device (e.g., a receiver, a smart phone, a computer, etc.), a medication delivery device, a pen cap, or a combination thereof.

In some aspects, outputting the recommendation can include outputting a recommended therapy. In some aspects, outputting the recommended therapy can include communicating the recommended therapy to a display device, a remote device (e.g., a receiver, a smart phone, a computer, etc.), a medication delivery device, a pen cap, or a combination thereof.

In some aspects, the method can further include administering the recommended dose and/or recommended therapy by a medication delivery device. In some aspects, administering the recommended dose and/or recommended therapy can be automatically administered or administered upon user confirmation.

In some aspects, first therapy information can include information about basal insulin recommended dose amounts, actual basal insulin dose amounts, timing of basal insulin administration, or a combination thereof.

In some aspects, alternatively, the method can further include outputting an indication that titration has stopped when overbasalization is determined and/or an indication that the patient should talk to their doctor when overbasalization is determined, rather than the recommendation to add the second therapy. For example, the indication can provide a notice to a user (e.g., “Titration has stopped as it is unable to optimize your basal insulin. Please talk to your doctor.”, “Talk to your doctor about overbasalization.”).

In some aspects, the one or more glucose metrics can include average glucose, median glucose, glucose time-in-range (TIR), glucose time below range (TBR), glucose time above range (TAR), glucose time very low (TVL), glucose management indicator (GMI), minimum morning glucose (MMG), minimum post-dose glucose (MPDG), post-prandial glucose (PPG), bedtime to AM glucose (BeAM), time in tight range (TITR), time in very tight range (TIVTR), or a combination thereof.

In some aspects, MMG can be configured to estimate a fasting glucose with continuous glucose monitoring (CGM) data. In some aspects, MMG can be calculated based on the lowest glucose value during the morning, for example, between about 4:30 AM and about 10:00 AM, at least about 30 minutes before an estimated wake time. In some aspects, MMG can be calculated based on a median MMG that omits days with overnight activity (e.g., treating hypoglycemia, taking a RA dose, missing a LA dose, etc.) from the calculation, thereby providing a more robust estimate of overnight glucose. In some aspects, MMG can be utilized as an effective metric for identifying patients in need of a LA insulin therapy adjustment (e.g., MMG 14-days prior to a LA insulin therapy setting increase and/or decrease). In some aspects, MMG can be utilized as a sensitive indicator of nocturnal hypoglycemia compared to a more traditional fasting glucose estimate (e.g., pre-breakfast glucose). In some aspects, a change in median MMG can indicate a change (e.g., increase or decrease) in LA insulin therapy. In some aspects, an increase in median MMG (e.g., 110 mg/dL to 155 mg/dL) can indicate an increase in LA insulin therapy. In some aspects, a decrease in median MMG (e.g., 155 mg/dL to 110 mg/dL) can indicate a decrease in LA insulin therapy.

In some aspects, the first therapy information can be manually entered via an input of a computing device. In some aspects, the first therapy information can be collected by a medication delivery device and can be communicated to a computing device. In some aspects, the first therapy information can be collected by a dose detection device for a medication delivery device, such as an add-on module, or smart pen cap, among others. In some aspects, the medication delivery device may include an infusion pump, a patch pump, or an injection pen, among others. In some aspects, an initial dose can also be calculated from data collected from one or more smart scales, other glucose measurement devices, or a combination thereof.

In some aspects, overbasalization can be determined based on a ratio of total daily dose (TDD) of insulin to a body weight of the user. In some aspects, overbasalization can be determined based on glucose variability. In some aspects, overbasalization can be determined based on a hypoglycemia metric. In some aspects, overbasalization can be determined based on a change in glucose level in a meal period or an overnight period.

In some aspects, the method can further include decreasing the basal insulin dose when the second therapy is added.

In some aspects, the second therapy can include a glucagon-like peptide-1 (GLP-1) receptor agonist. In some aspects, the method can further include titrating a dose of the GLP-1 receptor agonist. In some aspects, titrating the dose can include receiving a user input on side-effects of the second therapy. In some aspects, titrating the dose can include recommending an increased dose when the user input is associated with no side-effects. In some aspects, titrating the dose can include receiving a body weight measurement of the user. In some aspects, titrating the dose can include recommending an increased dose when the body weight measurement is above a target body weight.

In some aspects, the one or more glucose metrics can include a post-meal glucose rise for each of a plurality of meals. In some aspects, the method can further include recommending initiating a prandial insulin dose for a first meal when the post-meal glucose rise for one meal exceeds a threshold. In some aspects, the threshold can be defined by a total change (delta between minimum and maximum) in glucose level from a pre-meal period to a post-meal period, a rate of change (slope) in glucose level, a postprandial-preprandial differential, or a combination thereof. In some aspects, the method can further include recommending initiating a prandial insulin dose for a plurality of meals when the post-meal glucose rise for each of the plurality of meals exceeds a threshold. In some aspects, the threshold can be defined by a total change (delta between minimum and maximum) in glucose level from a pre-meal period to a post-meal period for each of the plurality of meals, a rate of change (slope) in glucose level for each of the plurality of meals, a postprandial-preprandial differential for each of the plurality of meals, or a combination thereof.

In some aspects, a system of therapy escalation for patients with diabetes can include an in vivo glucose monitoring device, a remote device, a medication delivery device, and a processor. In some aspects, the in vivo glucose monitoring device can be configured to measure glucose data of a user. In some aspects, the remote device can be in communication with the in vivo glucose monitoring device. In some aspects, the remote device can be configured to receive or retrieve glucose data from the in vivo glucose monitoring device. In some aspects, the medication delivery device can be in communication with the in vivo glucose monitoring device and the remote device. In some aspects, the medication delivery device can be configured to administer one or more dosing regimen. In some aspects, the processor can be in communication with the analyte measurement system, the remote device, and the medication delivery device. In some aspects, the processor can be coupled to a memory storing instructions that when executed cause the processor to perform operations including receiving glucose data of the user from the in vivo glucose monitoring device. In some aspects, the operations can further include receiving first therapy information of a first therapy from the remote device, the medication delivery device, or both. In some aspects, the first therapy can include basal insulin. In some aspects, the operations can further include calculating one or more glucose metrics based on the received glucose data. In some aspects, the operations can further include titrating a dose of the basal insulin based on the one or more glucose metrics. In some aspects, the operations can further include determining overbasalization based on one or more of the glucose data and the first therapy information.

In some aspects, the operations can further include outputting a recommendation to the remote device to add a second therapy when overbasalization is determined.

In some aspects, outputting the recommendation can include displaying the recommendation on the remote device (e.g., a receiver, a smart phone, a computer, etc.). In some aspects, displaying the recommendation can include displaying the recommendation on a display device, the remote device, a medication delivery device, a pen cap, or a combination thereof.

In some aspects, outputting the recommendation can include outputting a recommended therapy. In some aspects, outputting the recommended therapy can include communicating the recommended therapy to a display device, the remote device (e.g., a receiver, a smart phone, a computer, etc.), a medication delivery device, a pen cap, or a combination thereof.

In some aspects, the operations can further include administering the recommended dose and/or recommended therapy by a medication delivery device. In some aspects, administering the recommended dose and/or recommended therapy can be automatically administered or administered upon user confirmation.

In some aspects, a system for managing therapy to maintain one or more user metrics at or within a range of one or more targets can include an analyte measurement system, a remote device, a medication delivery device, and a software application. In some aspects, the analyte measurement system can be configured to measure an analyte of the user (e.g., glucose). In some aspects, the analyte measurement system can include an analyte sensor. In some aspects, the remote device can be in communication with the analyte measurement system. In some aspects, the remote device can be configured to receive or retrieve sensor data from the analyte sensor. In some aspects, the medication delivery device can be in communication with the analyte measurement system and the remote device. In some aspects, the medication delivery device can be configured to administer one or more dosing regimen. In some aspects, the software application can be in communication with the analyte measurement system, the remote device, and the medication delivery device.

In some aspects, the software application can be run on a processor coupled to a memory storing instructions that when executed cause the processor to perform operations that include, if the one or more user metrics is outside of the one or more targets, recommending a first dosing regimen (e.g., basal insulin). In some aspects, the operations can further include determining overbasalization of the first dosing regimen. In some aspects, the operations can further include, if the one or more user metrics remains outside of the one or more targets or if overbasalization is determined, adding a second dosing regimen (e.g., GLP-1, a single prandial dose, a prandial dose for each meal). In some aspects, the operations can further include determining overbasalization of the second dosing regimen. In some aspects, the user can be on an initial dosing regimen (e.g., GLP-1, etc.) prior to the first dosing regimen (e.g., basal insulin).

In some aspects, the one or more user metrics can include average glucose, median glucose, glucose time-in-range (TIR), glucose time in tight range (TITR), glucose time in very tight range (TIVTR), glucose time below range (TBR), glucose time above range (TAR), glucose time very low (TVL), glucose coefficient of variation (CV), glucose management indicator (GMI), minimum morning glucose (MMG), minimum post-dose glucose (MPDG), post-prandial glucose (PPG), basal bolus ratio (BBR), bedtime to AM glucose (BeAM), insulin sensitivity factor (ISF), post rapid-acting dose with correction glucose (PRA-CG), body weight, BMI, total daily dose (TDD) (e.g., of insulin), ratio of TDD to body weight, A1C level (e.g., percentage of glycated hemoglobin, mmol/mol), heart rate, blood pressure, or a combination thereof.

In some aspects, the one or more targets can include a target glucose level (e.g., about 110 mg/dL), a target glucose level range (e.g., about 110 mg/dL to about 155 mg/dL), a target basal state (e.g., MMG of about 100 mg/dL to about 130 mg/dL), a target meal state (e.g., MPDG of about 100 mg/dL to about 120 mg/dL), a target correction ISF state (e.g., PRA-CG of about 70 mg/dL to about 180 mg/dL), a target body weight, a target A1C level (e.g., 5.7%), a target heart rate, a target blood pressure, a target body mass, or a combination thereof. In some aspects, the one or more targets can be based at least in part on a body mass index (BMI) of the user and a mean glucose level of the user.

In some aspects, in the second dosing regimen, the operations can further include monitoring of one or more user compliance metrics for a first period of time. In some aspects, the one or more user compliance metrics can include properly wearing and using a CGM, properly and consistently administering medication, or a combination thereof. In some aspects, in the second dosing regimen, the operations can further include rapidly titrating the basal analog, GLP-1 class medication, or the mealtime RA insulin for a second period of time. In some aspects, rapidly titrating can include recommending an adjusted dose at a first interval (e.g., every day) based on the one or more user metrics for that first interval until no changes are made over a predetermined period of time (e.g., 3 days). In some aspects, in the second dosing regimen, the operations can further include monitoring of no change in dosing output states for a third period of time. In some aspects, in the second dosing regimen, the operations can further include maintaining titrating the basal analog, GLP-1 class drug, or the mealtime RA insulin for a fourth period of time. In some aspects, maintaining titrating can include recommending an adjusted dose at a second interval (e.g., several days, a week) based on the one or more user metrics for that second interval until a new issue is identified (e.g., new medication regimen, new exercise routine, new diet, etc.).

In some aspects, the software application can be configured to determine overbasalization based at least in part on medication delivery information. In some aspects, overbasalization may be determined based at least in part on a ratio of TDD of insulin to a body weight of the user exceeding a predetermined threshold. In some aspects, for example, the predetermined threshold can be about 0.5 units (U)/kg/day. In some aspects, the software application can be configured to determine overbasalization based at least in part on glucose metrics. In some aspects, overbasalization can be determined based at least in part on an increase in morning (AM)-bedtime (PM) differential and/or postprandial-preprandial differential. In some aspects, the software application can be configured to determine overbasalization based at least in part on a hypoglycemia metric succeeding a predetermined threshold. In some aspects, for example, the predetermined threshold can be a glucose concentration of about 70 mg/dL. In some aspects, the software application can be configured to determine overbasalization based at least in part on a glucose variability exceeding a predetermined threshold. In some aspects, for example, the predetermined threshold can include pre-meal rises below about 30 mg/dL, post-meal spikes below about 110 mg/dL, a total change (delta between minimum and maximum) in glucose level from a pre-meal period to a post-meal period, or a combination thereof. In some aspects, the software application can be configured to determine overbasalization based at least in part on a ratio of TDD of insulin to a body weight of the user exceeding a predetermined threshold (e.g., a basal insulin dose of greater than 0.5 U/kg/day), an increase in morning (AM)-bedtime (PM) differential and/or postprandial-preprandial differential, a hypoglycemia metric subceeding a predetermined threshold (e.g., a glucose concentration of about 70 mg/dL), an increase in variability of sensor data, or a combination thereof.

In some aspects, the operations can further include, if the one or more user metrics remains outside the one or more targets, recommending adding a second dosing regimen of glucagon-like peptide-1 (GLP-1) receptor agonists or dual gastric inhibitory peptide (GIP)/GLP-1 receptor agonists. In some aspects, the operations can further include, if overbasalization is determined, recommending adding the second dosing regimen of GLP-1 receptor agonists or dual GIP/GLP-1 receptor agonists.

In some aspects, the operations can further include, if the one or more user metrics remains outside the one or more targets, recommending adding a third dosing regimen of prandial insulin with one meal. In some aspects, the operations can further include, if overbasalization is determined, recommending adding the third dosing regimen of prandial insulin with one meal.

In some aspects, the operations can further include, if the one or more user metrics remains outside the one or more targets, recommending adding a fourth dosing regimen of multiple daily injections (MDI) of prandial insulin. In some aspects, the operations can further include, if overbasalization is determined, recommending adding the fourth dosing regimen of MDI of prandial insulin.

In some aspects, the operations can further include, if the one or more user metrics remains outside the one or more targets, recommending adding a fifth dosing regimen of basal insulin and prandial insulin with each meal. In some aspects, the operations can further include, if overbasalization is determined, recommending adding the fifth dosing regimen of basal insulin and prandial (bolus) insulin with each meal.

In some aspects, for any of the dosing regimen described above, the operations can further include performing a monitoring phase, a rapid titration phase, and a maintenance phase for the current dosing regimen. In some aspects, the monitoring phase can include monitoring one or more user compliance metrics of the current dosing regimen over a predetermined period of time (e.g., 3 days) to determine whether the user is properly wearing and using a CGM and properly and consistently administering medication. In some aspects, the one or more user compliance metrics can include frequency of glucose data (scans), gaps in glucose data, time of dose, size of dose (e.g., corresponding to recommended dose), receipt of user input, or a combination thereof. In some aspects, once the user compliance metrics are met, the rapid titration phase can include recommending an adjusted dose at a first interval (e.g., every day at midnight) based on the one or more user metrics for that first interval until no changes are made over a predetermined period of time (e.g., 3 days). In some aspects, dose adjustments can be at fixed intervals (e.g., 0.25 U increase, 10% increase, etc.) or can be proportional to the degree of glycemic dysfunction (e.g., higher dose changes if significantly outside glucose targets). In some aspects, the maintenance titration phase can include recommending an adjusted dose at a second interval (e.g., several days, a week) based on the one or more user metrics for that second interval until a new issue is identified (e.g., new medication regimen, new exercise routine, new diet, etc.)

In some aspects, the software application can be configured to adjust a dose amount of the one or more dosing regimen based at least in part on one or more metrics of the user. In some aspects, the one or more metrics can include average glucose, glucose TIR, glucose TBR, glucose TVL, GMI, MMG, PPG, BBR, BeAM, ISF, or a combination thereof.

In some aspects, the system can further include an external scale in communication with the software application. In some aspects, the external scale can be configured to measure a body weight of the user. In some aspects, the external scale can be configured to provide a body weight of the user to the software application to determine overbasalization based at least in part on a ratio of TDD of insulin to the measured body weight of the user exceeding a predetermined threshold (e.g., a basal insulin dose of greater than 0.5 U/kg/day). In some aspects, the remote device may prompt the user to enter a body weight measurement, or to weigh themselves using the external scale.

In some aspects, the system can further include an external sensor in communication with the software application. In some aspects, the external sensor can be configured to measure one or more user parameters. In some aspects, the one or more user parameters can include a meal parameter, an exercise related parameter, an activity parameter, a breathing parameter, a heart rate, a heart rate variability, temperature, blood pressure, a sleep related parameter, a nausea parameter, or a combination thereof. In some aspects, the external sensor can include an accelerometer, a gyroscope, a micro-electromechanical system (MEMS) device, a position sensor, a sound sensor, a smart device, a smart phone, a smart ring, a bed sensor, a smart pill bottle, a smart injection pen cap, or a combination thereof.

In some aspects, the operations can further include receiving an input signal from the user or a third party. In some aspects, the operations can further include sending a control signal to the software application to recommend adjusting one or more dosing regimen based at least in part on the input signal. In some aspects, the input signal can include feedback, clinician guidelines, user adherence, verbal instructions, a therapy pathway, a therapy escalation, a therapy session, historical therapy session analytics, or a combination thereof. In some aspects, the third party can include a medical professional, such as a clinician, a primary care physician (PCP), or a health care professional (HCP), among others.

In some aspects, the operations can further include performing analytics of sensor data and user data to generate a predictive model. In some aspects, the predictive model can be based on regression, model-based parameter adaptation, supervised machine learning, unsupervised machine learning, neural networks, classification models, or a combination thereof.

In some aspects, the software application can be configured to receive one or more measured parameters of the user over time from a remote server. In some aspects, the software application can be configured to recommend adjustment of one or more dosing regimen based at least in part on an algorithm or function incorporating the one or more measured parameters of the user over time. In some aspects, the software application can be configured to receive a constructed user profile. In some aspects, the constructed user profile can be generated using an artificial intelligence (AI) module trained using analytics from the remote server. In some aspects, the software application can be configured to recommend adjustment of the one or more dosing regimen based at least in part on the constructed user profile.

In some aspects, the processor can be part of the analyte measurement system, the remote device, or the medication delivery device. In some aspects, the software application can be part of an external device, a remote server, or a cloud server. In some aspects, the software application (e.g., one or more algorithms) can be executed by a combination of the devices.

In some aspects, communication between the analyte measurement system and the remote device can include wireless communication, near-field communication (NFC), Bluetooth, Bluetooth Low Energy (BLE), or a combination thereof.

In some aspects, a method of therapy escalation for patients with diabetes to detect non-adherence to a basal insulin recommendation can include receiving glucose data of a user from an in vivo glucose monitoring device. In some aspects, the method can further include recommending a first dose of basal insulin to the user. In some aspects, the method can further include calculating one or more glucose metrics based on the received glucose data. In some aspects, the one or more glucose metrics can include a minimum glucose metric. In some aspects, the method can further include titrating a recommended dose of basal insulin based on the one or more glucose metrics. In some aspects, the method can further include recommending a second dose of basal insulin to the user. In some aspects, the second dose can be different from the first dose, for example, due to titrating the recommended dose. In some aspects, the method can further include calculating a change in the minimum glucose metric from a first time period prior to recommending the second dose to a second time period after recommending the second dose. In some aspects, the method can further include determining whether the change in the minimum glucose metric is outside a predetermined metric. In some aspects, the method can further include outputting, if the change in the minimum glucose metric is outside the predetermined metric, an indication that titration of the basal insulin will stop.

In some aspects, the minimum glucose metric can include fasting glucose, daily minimum glucose (DMG), minimum morning glucose (MMG), average glucose for a predetermined time period, daily minimum hourly average glucose (DMHAG), or a combination thereof. In some aspects, for example, the minimum glucose metric can include DMHAG.

In some aspects, the predetermined metric can include the change in the minimum glucose metric being less than a predetermined percentage of an expected change of the minimum glucose metric. In some aspects, for example, the change in the minimum glucose metric can be less than 50% of an expected change of the minimum glucose metric. In some aspects, the expected change of the minimum glucose metric is based on one or more user metrics. In some aspects, the one or more user metrics can include body weight, BMI, age, insulin sensitivity factor (ISF), total daily dose (TDD), ratio of TDD to body weight, A1C level, heart rate, blood pressure, or a combination thereof. In some aspects, for example, the one or more user metrics can include ISF. In some aspects, the expected change of the minimum glucose metric can be based on historical glucose data of the user. In some aspects, the expected change of the minimum glucose metric can be proportional to a change between the first dose and the second dose.

In some aspects, the predetermined metric can include the change in the minimum glucose metric is less than a predetermined change in the glucose level. In some aspects, the predetermined metric can include the change in the minimum glucose metric is less than 5 mg/dL. In some aspects, the predetermined metric can include the change in the minimum glucose metric is less than 15 mg/dL. In some aspects, the predetermined metric can include the change in the minimum glucose metric is less than 10 mg/dL. In some aspects, the predetermined metric can include the change in the minimum glucose metric is less than 3 mg/dL. In some aspects, the predetermined metric can include the change in the minimum glucose metric is less than 1 mg/dL. In some aspects, the predetermined metric can include the change in the minimum glucose metric is less than 0.1 mg/dL. In some aspects, the predetermined metric can include the change in the minimum glucose metric is less than a range of about 0.1 mg/dL to about 15 mg/dL.

In some aspects, the predetermined metric can include determining a change in the minimum glucose metric based on a statistical method. In some aspects, the predetermined metric can include a statistical test of the minimum glucose metric that does not support a hypothesis that the minimum glucose metric has changed. In some aspects, the statistical test can include an insulin sensitivity test (IST), an insulin tolerance test (ITT), an oral glucose tolerance test (OGTT), a fasting plasma glucose (FPG) test, a random plasma glucose test, or a combination thereof.

In some aspects, the method can further include prompting the user to confirm administration of the second dose. In some aspects, the method can further include, if the user confirms administration of the second dose, resuming titration. In some aspects, the method can further include recommending, if the user does not confirm administration of the second dose, a third dose based on titrating the second dose.

In some aspects, the method can further include recommending limiting a change in dose amount of basal insulin if there is no change in the minimum glucose metric after titrating the recommended dose for a predetermined time period. In some aspects, limiting a change in dose amount can include capping the change in dose amount to no more than a predetermined percentage of the previous dose amount, for example, no more than 1%, 2%, 5%, 10%, etc. In some aspects, limiting a change in the dose amount can include stopping any change in the dose amount. In some aspects, the predetermined time period can include at least 3 days.

In some aspects, the method can further include stopping upward titration of basal insulin if the minimum glucose metric does not decrease after titrating the recommended dose for a predetermined time period. In some aspects, the method can further include stopping downward titration of basal insulin if the minimum glucose metric does not increase after titrating the recommended dose for a predetermined time period.

In some aspects, the method can further include activating a blinded mode such that glucose data is not displayed to the user.

In some aspects, the method can further include initiating, if the change in the minimum glucose metric is outside the predetermined metric, a counter configured to monitor a number of titration cycles. In some aspects, the method can further include stopping titration of basal insulin if the change in the minimum glucose metric remains outside the predetermined metric after a predetermined number of titration cycles with increasing or decreasing doses of basal insulin.

In some aspects, the method can further include receiving basal insulin dose administration time data. In some aspects, titrating the recommended dose is not based on basal insulin dose amount data.

In some aspects, the method can further include outputting a prompt to the user to confirm the user is following the recommended dose. In some aspects, the method can further include outputting a prompt to the user to have the user seek guidance from a health care professional. In some aspects, the method can further include outputting a quiz to the user to verify the user is following the recommended dose.

In some aspects, a method of detecting non-adherence to a dose recommendation can include receiving glucose data of a user in a first time period from an in vivo glucose monitoring device. In some aspects, the method can further include determining a first minimum glucose metric for the first time period. In some aspects, the method can further include providing a dose recommendation to the user based on the glucose data in the first time period. In some aspects, the method can further include receiving glucose data of the user in a second time period following the dose recommendation. In some aspects, the method can further include determining a second minimum glucose metric for the second time period. In some aspects, the method can further include determining non-adherence to the dose recommendation based on a comparison of the first and second minimum glucose metrics. In some aspects, the method can further include outputting an indication of non-adherence.

In some aspects, the first and second minimum glucose metrics can include daily minimum hourly average glucose (DMHAG).

In some aspects, the comparison can include a change between the first and second minimum glucose metrics. In some aspects, determining non-adherence comprises comparing the change between the first and second minimum glucose metrics to a predetermined percentage change threshold. In some aspects, determining non-adherence comprises comparing the change between the first and second minimum glucose metrics to a predetermined change in the glucose level.

In some aspects, the comparison can include a direction of the first and second minimum glucose metrics. In some aspects, the direction is inversely proportional to the dose recommendation. In some aspects, for example, non-adherence of the user can be determined by the direction (e.g., trend) of first and second minimum glucose metrics increasing (not decreasing) in response to an increase in the dose recommendation. In some aspects, for example, non-adherence of the user can be determined by the direction (e.g., trend) of first and second minimum glucose metrics decreasing (not increasing) in response to a decrease in the dose recommendation.

Implementations of any of the techniques described above may include a system, a method, a process, a device, and/or an apparatus. The details of one or more implementations are set forth in the accompanying drawings and the description below. Other features will be apparent from the description and drawings, and from the claims.

Further features and exemplary aspects of the present disclosure, as well as the structure and operation of various aspects, are described in detail below with reference to the accompanying drawings. It is noted that the aspects are not limited to the specific aspects described herein. Such aspects are presented herein for illustrative purposes only. Additional aspects will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate the aspects and, together with the description, further serve to explain the principles of the aspects and to enable a person skilled in the relevant art(s) to make and use the aspects.

FIG. 1 shows an example of a process for guiding a PWD in managing their insulin therapy.

FIG. 2 shows an example of the pre-training survey of the process of FIG. 1.

FIG. 3 shows an example of a patient messaging profile that can be created in the process of FIG. 1.

FIG. 4 shows an example of a system for diabetes management.

FIG. 5 shows an example of a system for guiding a PWD in managing their insulin therapy.

FIG. 6 shows an example of a state diagram that can be used by the insight generation engine of FIG. 5.

FIG. 7 schematically shows a prioritization of insights that can be performed by the insight generation engine of FIG. 5.

FIGS. 8A-8B show an example of a process that can be performed by the insight generation engine of FIG. 5.

FIG. 9 shows an example of multimodal insight triggering.

FIG. 10 shows an example of multimodal insight satisfaction evaluation.

FIG. 11 shows an example of actions relating to an insight.

FIG. 12 shows an example of a computing device that can be used to implement the techniques described here.

FIG. 13 is a schematic illustration of a therapy management system, according to an exemplary aspect.

FIG. 14A is a schematic control diagram for therapy escalation pathways, according to an exemplary aspect.

FIG. 14B is a schematic therapy assessment for therapy escalation pathways, according to an exemplary aspect.

FIG. 14C is a schematic control diagram for therapy escalation pathways, according to an exemplary aspect.

FIG. 15 is a schematic control diagram for a first dosing regimen, according to an exemplary aspect.

FIG. 16 is a schematic control diagram for a second dosing regimen, according to an exemplary aspect.

FIG. 17 is a schematic control diagram for a third dosing regimen, according to an exemplary aspect.

FIG. 18 is a schematic control diagram for a fourth dosing regimen, according to an exemplary aspect.

FIG. 19A is a schematic illustration of basal states, according to an exemplary aspect.

FIG. 19B is a schematic illustration of meal states, according to an exemplary aspect.

FIG. 19C is a schematic illustration of correction insulin sensitivity factor (ISF) states, according to an exemplary aspect.

FIG. 20 illustrates a flow diagram for therapy escalation for patients with diabetes, according to an exemplary aspect.

FIG. 21 illustrates a flow diagram to detect non-adherence to a dose recommendation, according to an exemplary aspect.

FIG. 22 illustrates a flow diagram to detect non-adherence to a dose recommendation, according to an exemplary aspect.

FIG. 23 is a schematic illustration of a computing device that can be used to implement the operations described herein, according to an exemplary aspect.

The features and exemplary aspects of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements. Additionally, generally, the left-most digit(s) of a reference number identifies the drawing in which the reference number first appears. Unless otherwise indicated, the drawings provided throughout the disclosure should not be interpreted as to-scale drawings.

### DETAILED DESCRIPTION

This document describes examples of systems and techniques that use reinforcement learning as a way of applying and obtaining the benefits of machine learning technology in managing insulin therapy for a PWD. Generally, the reinforcement learning can be applied to aspects of the insulin-therapy management process where the algorithm does not have a prespecified action to perform, so that indirect feedback can be taken into account in planning the action(s). A system according to the present disclosure is better at communicating with the PWD over the course of the insulin therapy because it takes into account a broad scope of the PWD's current physiological and psychological being (sometimes referred to as considering the complete state of the PWD.)

Provided herein are system, apparatus, device, method, and/or computer program product aspects, and/or combinations and sub-combinations thereof, for therapy escalation pathways to manage diabetes and maintain euglycemia, such as by maintaining one or more glucose metrics at a target level or in a target range.

A system as described below can continuously monitor a glucose level of a user, determine one or more glucose metrics, implement one or more dosing regimen, confirm user adherence to the therapy, determine if any overbasalization exists for the therapy, monitor the therapy over discrete periods of time, and adjust and/or escalate the therapy based on one or more measured user metrics in response to the one or more dosing regimen.

This specification discloses one or more aspects that incorporate the features of this present disclosure.

The aspect(s) described, and references in the specification to “one aspect,” “an aspect,” “an example aspect,” “an exemplary aspect,” etc., indicate that the aspect(s) described may include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it is understood that it is within the knowledge of one skilled in the art to effect such feature, structure, or characteristic in connection with other aspects whether or not explicitly described.

The term “about” or “substantially” or “approximately” as used herein indicates the value of a given quantity that can vary based on a particular technology. Based on the particular technology, the term “about” or “substantially” or “approximately” can indicate a value of a given quantity that varies within, for example, 1-15% of the value (e.g., ±1%, ±2%, ±5%, ±10%, or ±15% of the value). In some aspects, the terms “substantially” and “about” used herein are used to describe and account for small fluctuations, such as due to variations in processing. For example, the terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. Also, when used herein, an indefinite article such as “a” or “an” means “at least one.”

Numerical values, including endpoints of ranges, can be expressed herein as approximations preceded by the term “about,” “substantially,” “approximately,” or the like. In such cases, other aspects include the particular numerical value. Regardless of whether a numerical value is expressed as an approximation, two aspects are included in this disclosure: one expressed as an approximation, and another not expressed as an approximation. It will be further understood that an endpoint of each range is significant both in relation to another endpoint, and independently of another endpoint.

Aspects of the disclosure may be implemented in hardware, firmware, software, or any combination thereof. Aspects of the disclosure may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, and/or instructions may be described herein as performing certain actions. However, it should be appreciated that such descriptions are merely for convenience and that such actions in fact result from computing devices, processors, controllers, or other devices executing the firmware, software, routines, instructions, etc.

The term “overbasalization” as used herein indicates the titration of basal insulin beyond an appropriate dose in an attempt to achieve glycemic targets. In some aspects, the appropriate dose is one that allows users to reach glucose targets.

The terms “long-acting (LA) insulin” and “basal insulin” as used herein are interchangeable terms and indicate a dose of insulin associated with non-meal times as well as overnight.

The terms “rapid-acting (RA) insulin”, “prandial insulin,” and “bolus insulin” as used herein are interchangeable terms and indicate a dose of insulin associated with a meal.

The term “therapy intervention” as used herein may refer to initiating or modifying a therapy regimen or recommending initiating or modifying a therapy (e.g., adjusting a dose amount, adjusting a type of dose, etc.) over a time period (e.g., adjustment on a daily and/or weekly basis).

The term “duration of intervention” may refer to a time between therapy interventions. For example, a duration of intervention can be over a predetermined period of time (e.g., daily, two days, a week, two weeks, etc.).

The term “monitoring phase” may refer to monitoring one or more user compliance metrics of a current therapy over a predetermined period of time (e.g., daily, two days, three days, etc.).

The term “titrating a dose” as used herein may refer to determining to maintain or adjust a dose amount (e.g., increasing or decreasing) to improve glucose control.

The term “upward titration” as used herein refers to increasing a dose amount.

The term “downward titration” as used herein refers to decreasing a dose amount.

The term “titration cycle” as used herein may refer to a cycle of recommending a first dose amount, calculating one or more glucose metrics, and maintaining or adjusting the administered first dose amount based on the one or more glucose metrics.

The term “add a second therapy” or “adding a second therapy” as used herein may refer to a medication therapy regimen including one or more therapies, and a second therapy can be added or incorporated into the medication therapy regimen such that the medication therapy regimen includes first and second therapies (e.g., basal insulin therapy and GLP-1 therapy, basal insulin therapy and prandial insulin therapy, among others).

The term “inversely proportional” as used herein may refer to two quantities that behave oppositely such that a first quantity increases with respect to a decrease in a second quantity or vice-versa. For example, a direction (e.g., trend) of first and second minimum glucose metrics decreases in response to an increase in a dose recommendation or vice-versa.

### Exemplary Management Process for Insulin Therapy

As discussed above, reinforcement learning can be applied to aspects of an insulin-therapy management process where the algorithm does not have a prespecified action to perform, so that indirect feedback can be taken into account in planning the action(s). A system according to the present disclosure is better at communicating with the PWD over the course of the insulin therapy because it takes into account a broad scope of the PWD's current physiological and psychological being (sometimes referred to as considering the complete state of the PWD.)

In some implementations, an insight engine driven by reinforcement learning can be provided that seeks to optimally guide the PWD in their insulin injection journey. The insight engine can use PWD preferences, health care provider direction, physiological data, behavioral data, drug dosing data, and measurements of outcomes for the PWD and other similar PWDs to select coaching messages and therapy adjustments. For example, a coaching message can instruct the PWD on how a change in their behavior could benefit their diabetes journey. As another example, a therapy adjustment can include any change in insulin therapy, such as to improve glucose performance (e.g., changing the type of insulin, changing the timing of taking insulin, changing a mental model for dosing of a type of insulin (e.g., long-acting insulin), changing a dose of insulin (e.g., long-acting or rapid-acting insulin), or changing a correction table for insulin). The primary goal of the insight engine can be to understand each patient and determine how best to help them achieve control of their diabetes. In the goal of meeting the PWD where they are in their journey, the system can leverage different approaches to assess their current state and provide clinically relevant recommendations whether they are initiating a new therapy, optimizing an existing therapy or driving recommendations for a change in therapy. The insight engine can use generalized reinforcement learning in guiding the PWD toward better therapy, including, but not limited to, with regard to insulin settings, measuring blood glucose levels, taking injections, and/or controlling the time of injections. System components can include, but are not limited to, blood glucose meters or monitors, insulin pumps, smart caps on injection pens, an insulin-therapy application, and/or cloud infrastructure.

The present disclosure can provide a management process for insulin therapy that includes initial interactions with the PWD, posing questions to the PWD and/or a health care provider to obtain preferences, training the PWD, subjecting the PWD to a high-touch interaction phase where insulin settings for the PWD can be rapidly adjusted based on standard guidelines, and finally conducting an ongoing phase that seeks to optimize the insulin therapy using reinforcement learning. The high-touch interaction phase and one or more other initial phases can provide the preparation for later optimization. In such a management process, the system or technique can take into account an overall view of the general state of the PWD, as opposed to only a specific measure. From such a state, the system/technique can probabilistically determine what action(s) to perform next (e.g., increase, decrease, or maintain the dose, and/or make another recommendation or perform coaching). After performing the action(s), the system/technique can observe one or more aspects regarding the PWD to evaluate whether it took the right action(s), thereby gaining the feedback as part of the reinforcement learning. In the future, if/when a similar situation occurs, the system/technique can take such feedback into account. For example, the feedback can make it more (or less) likely that the same action(s) be performed again.

Insulin delivery devices include, but are not limited to, insulin injection pens, insulin inhalers, insulin pumps, and insulin syringes. The improper dosing of insulin, whether due to human error, malfunction of an insulin pen, skipping doses, double dosing, or incorrect dosing, is always a concern. Methods, devices, and systems provided herein are described for the delivery of insulin, collection of blood glucose data, and/or the treatment of diabetes. Moreover, methods, devices, and systems provided herein may be adapted for the delivery of other medications, the collection of other analyte data, and/or the treatment of other diseases. Methods, devices, and systems provided herein are described by exemplifying features and functionalities of a number of illustrative embodiments. Other implementations are also possible.

Some examples herein refer to an insulin injection pen. An insulin injection pen includes at least one container holding insulin (e.g., an insulin cartridge), a dial or other mechanism to specify a dose, and a pen needle for transcutaneous delivery of the insulin into the tissue or vasculature of the person with diabetes. In a reusable insulin injection pen, the insulin container (e.g., the cartridge) is replaceable or refillable. A prefilled insulin injection pen is intended for use during a limited time. The dose-specifying mechanism can include a rotatable wheel coupled to mechanics and/or electronics for capping the administered amount of insulin at the volume of the specified dose (e.g., in terms of a number of units of insulin). The dose-specifying mechanism can have a mechanical and/or electronic display that reflects the current setting of the mechanism. The pen needle can be permanently attached to the housing of the insulin injection pen (e.g., as in the case with a disposable pen), or it can be removable (e.g., so that a new needle can be applied when needed). For example, a replaceable pen needle can include a hollow needle affixed to a fitting configured for removeable attachment to an end of the insulin injection pen.

Some examples herein refer to a mobile communication device. As used herein a mobile communication device includes, but is not limited to, a mobile phone, a smartphone, a wearable electronic device (e.g., a smart watch), a tablet, a laptop computer, a portable computer, and similar devices. A mobile communication device includes one or more processors, a non-transitory storage device (e.g., a memory and/or a hard drive) holding executable instructions for operating the mobile communication device, wireless communication components, and one or more input and/or output devices (e.g., a touchscreen, display, or keyboard). The mobile communication device can operate according to one or more application programs stored locally on the mobile communication device, or remotely (e.g., when using cloud computing), or combinations thereof. The mobile communication device can execute at least one operating system in order to perform functions and services.

Some examples herein refer to a blood glucose meter (BGM). A BGM is an electronic device configured to receive a sample from the PWD (e.g., a blood sample) and analyze the sample to estimate the blood glucose level of the person with diabetes. The BGM may be configured to have a new test strip partially inserted into the BGM for each sample, and the person then places a drop of blood onto the end of the test strip extending from the BGM. The BGM performs testing on the drop of blood through the test strip.

Some examples herein refer to a continuous glucose monitor (CGM). A CGM is an electronic device configured to take readings of glucose values on an ongoing basis or at regular intervals in order to estimate the blood glucose level of the PWD. A CGM can have an electrode placed under the skin that transmits its output to a receiver (e.g., via a transmitter), such as a handheld device. Another type of CGM can be fully implantable under the skin of the PWD. Yet another type of CGM can be non-invasive and avoid puncturing the skin, such as by analyzing the PWD's breath or by impinging light on the PWD's skin. Some CGMs determine blood glucose values periodically (e.g., after a certain number of seconds or minutes) and output the information automatically or upon being prompted. The CGM may include wireless communication components for one or more types of signaling, including, but not limited to, Near-Field Communication (NFC) and/or Bluetooth communication.

In some embodiments, the systems, devices, and/or methods provided herein can recommend insulin doses (e.g., dosages of long-acting and/or rapid-acting insulin) using any suitable technique. In some embodiments, recommended insulin dosages may be based upon blood glucose data (e.g., current estimated glucose value (EGV) from a CGM, flash glucose monitor, blood glucose meter, or any other sensor, blood glucose trend data, etc.), insulin administration data (bolus dosage amounts of rapid-acting insulin, dosages of long-acting insulin, dosage times, calculation of Insulin-on-Board (IOB) and/or active insulin, etc.), meal data (mealtimes, user estimated carbohydrates, user estimated meal categorizations, user estimated glycemic impact of meal user meal history, user meal trends, etc.), and/or one or more insulin delivery parameters (e.g., total daily dose of basal insulin or long-acting insulin, carbohydrate-to-insulin ratio (CR), insulin sensitivity factor (ISF), etc.). Methods, devices and systems provided herein can, in some embodiments, adjust insulin delivery parameters over time based on glucose data and/or insulin administration data.

Some examples herein refer to long-acting insulin and rapid-acting insulin, or in some cases more generally to first and second types of insulin. Insulin used for therapeutic treatment is often synthesized human insulin. Moreover, different insulins can be characterized in how quickly they typically begin to work in the body of the person with diabetes after administration, and/or how long they typically remain active in the body of the person with diabetes. Rapid-acting insulin can be used to dose for meals or to correct high blood sugar. There is more than one type of insulin that can be considered a rapid-acting insulin. Many, but not necessary all, rapid-acting insulins begin working within about one hour after administration. Similarly, there is more than one type of insulin that can be considered a long-acting insulin, and many, but not necessarily all, long-acting insulins begin working about one hour or longer after administration. Long-acting insulin is often referred to as basal insulin (e.g., insulin used to support basic metabolic needs). Generally, a long-acting insulin has a greater active time (i.e., the length of time that the insulin continues to be active in the body of the person with diabetes after administration) than a rapid-acting insulin. As such, a long-acting insulin is an example of a type of insulin having an active time that is longer than an active time for a type of insulin such as a rapid-acting insulin.

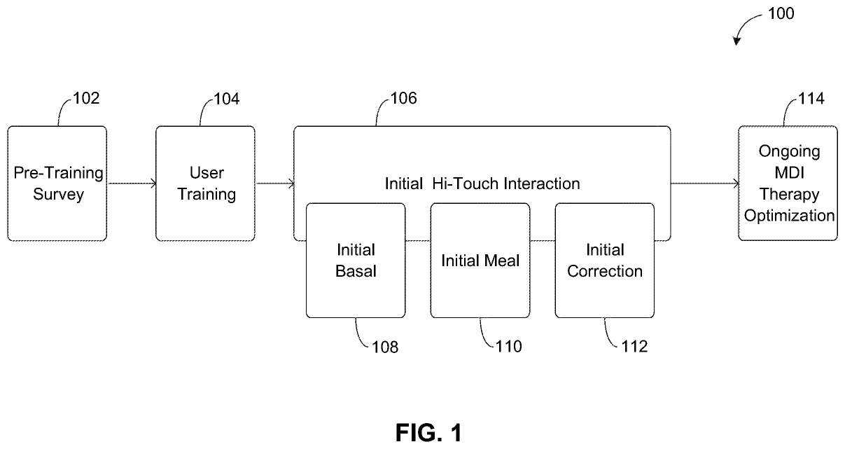

FIG. 1 shows an example of a process 100 for guiding a PWD in managing their insulin therapy. The process 100 can be used in combination with one or more other examples described elsewhere herein. The process 100 is schematically shown as multiple boxes. More or fewer operations than shown can be performed in some implementations.

A pre-training survey stage 102 involves collecting data from and/or about the PWD. The data can be collected by administering one or more surveys to the PWD. For example, the survey can be performed in person and/or presented on a display or using a speaker of a computer system (e.g., on a mobile electronic device). The survey can inquire about the goals of the PWD with regard to managing the insulin therapy and other aspects of the PWD's person or style. For example, the survey can probe how to communicate with PWD during the insulin therapy. The pre-training survey stage 102 can collect information from a health-care provider (HCP) of the PWD. For example, the HCP can be a physician, nurse, other health professional, or a family member or other caretaker. Information collected from the HCP can include information about the PWD's health or how the PWD should be treated. For example, the HCP can recommend pushing the PWD's health state to an optimal blood glucose level or modifying one or more aspects of the process 100. As another example, the pre-training survey stage 102 can seek to determine whether to apply a generic start regimen for the insulin therapy with the PWD, from a perspective of therapy sufficiency or messaging receptibility.

The process 100 can include a user training stage 104 that can be directed at the PWD and/or the PWD's HCP. In some implementations, the user training stage 104 provides education about diabetes and/or insulin therapy, and about other aspects of the process 100. The user training stage 104 can be performed in person and/or presented on a display or using a speaker of a computer system (e.g., on a mobile electronic device). The format and/or content of the user training stage 104 can be tailored using data or other information collected in the pre-training survey stage 102.

The process 100 can include a high-touch interaction phase 106. For example, “high-touch” can signify that the high-touch interaction phase 106 involves relative intensive, frequent, or otherwise substantial engagement with the PWD. In some implementations, the high-touch interaction phase 106 can be characterized as an initial phase, in that it precedes a subsequent phase of less intensive/frequent/substantial interaction with the PWD (to be described bow). The information collected in the pre-training survey stage 102 can change the contents of the high-touch interaction phase 106 or how the high-touch interaction phase 106 is conducted. For example, based on the collected information the process 100 may identify other PWDs who have medical and/or physiological characteristics similar to those of the present PWD and use their settings or other process configurations as starting points for the PWD. As another example, in the absence of other PWDs with similar characteristics the process 100 may use data obtained frow/for the PWD as the initial settings.

In some implementations, some or all aspects of the high-touch interaction phase 106 can be characterized as a titration with regard to the PWD and the PWD's insulin therapy. The titration can involve the use of multiple insights (to be exemplified below) where the intended action to be performed is the adjustment of the insulin therapy. Initially (e.g., within the high-touch interaction phase 106), the titration can involve a set of high-touch interactions with regard to the PWD within a relatively short period of time (e.g., days or weeks) after the beginning of the high-touch interaction phase 106 designed to establish the proper data and create conditions under which insulin therapy can be rapidly adjusted. An insight engine (to be exemplified below) can be designed to operate mostly on the available data, however the high-touch interaction phase 106 can be an exception. Rather, in the high-touch interaction phase 106, the PWD may be asked to log meals, wake up times, or whether they missed a dose. The process 100 can also or instead use other sources of information to augment these observations. For example, a wearable device can provide information for data collection.

The interactions during the high-touch interaction phase 106 can include the establishment of one or more insulin dose settings by repeated adjustment of the insulin therapy. For example, these can be considered initial settings for the purpose beginning an optimization procedure where the settings can be modified one or more times. This can be characterized as part of a titration with regard to the insulin dosage amounts of the PWB. Insulin settings can include, but are not limited to, a basal dose setting 108, a meal dose setting 110, and a correction dose setting 112.

In some implementations, the basal dose setting 108 of insulin for the PWD can be established by the broadly accepted and utilized American Diabetes Association (ADA) guidance, as an example of a publicly available guideline regarding insulin therapy. Another example of a guideline is the recommendations from the American Association of Clinical Endocrinologists (AACE). The guideline can be applied by directly utilizing blood glucose values from a blood glucose meter to understand the PWD's fasting plasma glucose (FPG) levels. The guideline can be applied by interpreting CGM values to reduce the burden on the user at this critical time in starting a new therapy. In some implementations, CGM can be used for assessing the minimum morning glucose (MMG) of the PWD. For example, MMG can be defined to be the lowest glucose value between 4:00 a.m. and 30 minutes prior to the first activity of the day. These values can be processed in one or more ways. For example, highs from treatments of overnight lows (via eating, etc.) can be excluded. As another example, mornings with overnight lows resulting in morning rebound highs can be excluded. As another example, adjustments can be made for having nonstandard awake hours (e.g., for shift workers). A basic, consistent and flexible framework for initiation can be created that is independent of the given glucose measurement tool. Building from the guidelines a PWD can be initiated at a dose of a predefined number of units, or of the specific amount indicated by the HCP, daily at bedtime. The system can recommend an increase of a specific number of units, or a certain percentage, at regular intervals (e.g., once every fixed number of days) until the FPG and/or the MMG falls within a target range. Other adjustments can be made. For example, the system can evaluate for overbasalization and can consider adjunctive therapies (e.g., if a basal dose is more than a predefined number of units/kg/day, elevated bedtime-morning (BeAM) glucose difference and/or post-preprandial differential, hypoglycemia (aware or unaware), and/or high variability (e.g., a coefficient of variation above a prespecified number).

In some implementations, the meal dose setting 110 of insulin for the PWD can be established in a step approach that focuses on one meal at a time. The meal dose setting 110 can take into account average measures of blood glucose levels (e.g., the A1C test), the basal dose setting 108, the FPG, and/or the MMG. Pre-meal glucose levels can be considered. The system can recommend an increase of a specific number of units, or a certain percentage, at regular intervals (e.g., once every fixed number of days) until the next meal's pre-meal glucose level falls within a target range. If hypoglycemia occurs, the system can review the blood glucose patterns and decrease the dose of insulin by a predefined amount.

In some implementations, the correction dose setting 112 of insulin for the PWD can be established to compensate for the average glucose level (e.g., AIC) not being within target range. The system can take into account an insulin sensitivity factor for the PWD.

As mentioned, some or all settings in the high-touch interaction phase 106 (e.g., the basal dose setting 108, meal dose setting 110, and/or correction dose setting 112) can be established based on a publicly available guideline regarding insulin therapy. This can be done after a HCP for the PWD confirms that there is no need for a personalized glycemic target for the PWD. Such guidelines can specify one or more target ranges for A1C (e.g., taking into account that the PWD is nonpregnant and does not have significant hypoglycemia), time in range target can be evaluated using an ambulatory glucose profile and/or a glucose management indicator, the system can accept AIC levels lower than goal based on HCP judgment and PWD preference while monitoring for significant hypoglycemia or other adverse effects, the system can apply less stringent goals (e.g., for AIC) when the harms of treatment are greater than the benefits. While the system can set the glycemic targets based on individual criteria, the overarching clinical targets can be defined as defaults. For example, this can include A IC levels, preprandial capillary plasma glucose levels, and/or peak postprandial capillary plasma glucose levels. More or less stringent glycemic goals may be appropriate for individual patients. A CGM can be used to assess glycemic targets.

The duration of the high-touch interaction phase 106 can be defined in any of multiple ways. In some implementations, a goal-based duration (e.g., a results-based duration) can be used. The system can determine that it has sufficient interactions with, and data about, the PWD so that the dose is the correct one. For example, the dose can be close enough to a correct level that a less interactive phase that relies on reinforcement learning can be initiated. A goal-based evaluation by the system can take into account that the blood glucose stays within target for at least a certain percentage of time, and that the PWD is taking insulin as recommended. In some implementations, a predetermined length of time (e.g., a fixed number of days) can be used.

The process 100 can include an ongoing phase 114. The ongoing phase 114 includes collecting performance metrics for the PWD, applying reinforcement learning to the performance metrics, and automatically performing an action with regard to the PWD based on the reinforcement learning. The ongoing phase 114 can be performed based on the survey information from the pre-training survey stage 102. For example, the specific insulin therapy recommendations, and/or ways of communicating with the PWD, can affect whether to continue monitoring the diabetes and watch for new developments, whether to intensively manage the diabetes, whether to minimize cost and expenses for the PWD, whether to maximize health benefits for the PWD, and how to talk to the PWD. In the ongoing phase 114, the system can look for behaviors and insulin therapy settings, and for the most impactful thing(s) to tell the PWD. For example, that message can be that the PWD is not taking enough doses, in lieu of recommending what each dose should be. As another example, the PWD can be recommended to take doses earlier or later. In some implementations, one or more insights can be evaluated and lead to performance of at least one action. The action can include a communication to the PWD (or to a HCP), and/or a change in a setting regarding the insulin therapy. What message to generate to the PWD can be chosen in view of their preferences. After acting on an insight, the insight can be placed in monitoring for a period of time. If the condition(s) should improve during that time, the PWD can be informed and the monitoring terminated. If there is no improvement, other remediation can be considered.

The ongoing phase 114 can include one or more ongoing therapy adjustment periods. An ongoing therapy adjustment period can be intended to adapt to the needs of the PWD through the rest of their journey with multiple daily injections (MDI) of insulin. The ongoing therapy adjustment period(s) can be built around examination of performance metrics that are created to understand the behavioral, physiological, and drug-dosing behavior of the PWDs. These metrics can be as current as possible, such as being updated between once a day and once an hour. For example, the performance metrics are not intended to be real-time, and many observations are looking for trends over the recent past, not sudden or real-time changes. An insights generation engine (to be exemplified below) can evaluate insights sequentially to determine which insights, if any, should be acted upon. The most significant negative insights can be considered, and only the most relevant (i.e., likely to produce positive results) insights may be presented to the PWD. The most positive celebrations can be considered. The insights are triggered and actions may be taken (e.g., a coaching message can be sent to the PWD or the therapy can be adjusted); then, observation periods can begin and a cooldown period can begin. The observation period is a period during which the system can look for improvements. The criteria to know that an insight has improved can be, but are not necessarily, the same as the one or more criterion to trigger the insight in the first place. The system can evaluate other criteria or use purpose-built classification methods to determine if the PWD has improved based on the actions taken. The cooldown period can be used to ensure that the system does not act on insights that were recently acted upon. Safety-related insights (e.g., involving hypoglycemia) can be the highest priority and have very low to no cooldowns. Insights may not coach safety issues, but rather may coach non-urgent issues that tend to reduce insulin based on excessive hypoglycemia observed despite normal or low insulin delivery.

Titration and coaching can both be included in this framework. For example, titration has an action that primarily changes insulin therapy settings. As another example, coaching primarily has an action that instructs PWDs in how to improve their behavior (or instructs them in where they can get sensors or insulin if that is their problem).

The action/coaching steps may push questions to PWDs that help the system better coach or instruct. For example, a communication can state “We've noticed you haven't been wearing your sensor. Do you need help with (a) an Rx (b) affording sensors (c) dealing with sensor failures/sensors being knocked off (d) sensor adhesive allergic reaction (d) I'm taking a break from sensors.” The answer can help craft and shape the coaching. “We've noticed you haven't been taking your <long acting insulin brand>? How can we help you (a) an Rx (b) obtaining affordable insulin.” The PWD can make inputs into the system (e.g., using a device to enter text or choose between options, or by speaking into a microphone).

As actions are taken, an observation period can be implemented. The observation period can facilitate evaluation of how effective the action taken was in improving the PWDs journey (PWD user state). By evaluating how effective messaging/actions were, the system can change how likely the system will be to take a given action in the future. For example, changes to messaging preference (for example, the PWD responds to messages about cost) will cause future messages to be more likely to focus on those areas that worked in the past. Similarly, the thresholds, cooldown periods, and observation period parameters can be adaptive in response to successful actions.

Parameter adjustments can also or instead be adapted based on similar patients. In some implementations, a given user may not need many insulin titration events after the initial titration period. However, if similar patients are seeing success from titration actions, the system may adjust the parameters for all similar patients to take advantage of this learning. Other aspects than titration can also or instead be learned from the data of another patient. This can include, but is not limited to, coaching messages and/or the balance between basal and bolus doses.

The ongoing phase 114 can include one or more adjustments of one or more of the basal dose setting 108, the meal dose setting 110, and/or the correction dose setting 112. In some implementations, the process 100 can recommend an increase, decrease, or no change in the basal dose setting 108 during the ongoing phase 114 considering one or more of: a mean glucose value during a specified time interval; a percentage of time in hypoglycemia and hyperglycemia during a specified time interval; a glucose rate of change calculated during a specified time interval; a hypoglycemia treatment occurring during a specified time interval; or hypoglycemia occurring more than a specified fraction of the time. The reinforcement learning can define positive and/or negative rewards for one or more circumstances. A goal state can be taken into account in whether to recommend an increase, decrease, or no change in the basal dose setting 108. For example, the goal state can be defined based on a mean night glucose level and no hypoglycemia or hyperglycemia. If more than a fixed percentage of days have hypoglycemia, then the system may only consider hypoglycemia days to find recommendations. If hypoglycemia is found to be triggered by dinner or late night snack bolus, then the system may ignore the hypoglycemia of that day in its evaluation. In some implementations, a positive reward can be generated to the system if the PWD reaches a goal state and/or has less or no change in hypoglycemia, hyperglycemia, or mean glucose error.

In some implementations, the process 100 can recommend an increase, decrease, or no change in the meal dose setting 110 during the ongoing phase 114 considering one or more of: a minimum glucose level during a specified time interval; a target glucose level; a glucose rate of change calculated during a specified time interval; hypoglycemia occurring more than a specified fraction of the time; no hypoglycemia occurring. The reinforcement learning can define positive and/or negative rewards for one or more circumstances. A goal state can be defined. For example, the goal state can include a postprandial error within a specified range and no hypoglycemia. In some implementations, a positive reward can be generated to the system if the PWD reaches a goal state and/or has less or no change in rate of change and postprandial error. In some implementations, a negative reward can be generated to the system if the hypoglycemia falls between a specified level. If more than a fixed percentage of days have hypoglycemia, then the system may only consider hypoglycemia days to find recommendations.

In some implementations, the process 100 can recommend an increase, decrease, or no change in the correction dose setting 112 during the ongoing phase 114 considering one or more of: a minimum glucose level during a specified time interval; a glucose rate of change calculated during a specified time interval; or a glycemic risk index. The reinforcement learning can define positive and/or negative rewards for one or more circumstances. A goal state can be defined. For example, the goal state can include a post-correction error within a specified range and no hypoglycemia. In some implementations, a positive reward can be generated to the system if the PWD reaches a goal state and/or has less or no change in rate of change and post-correction error. In some implementations, a negative reward can be generated to the system if the hypoglycemia falls between a specified level. If more than a fixed percentage of days have hypoglycemia, then the system may only consider hypoglycemia days to find recommendations.

The system can use various statistical tools in the evaluation during the ongoing phase 114. For example, a mean value and/or a median value can be used for one or more measured entities.

One or more considerations can be taken into account in evaluating whether to adjust overarching therapies. Considerations for escalation or less likely regression between therapies can be defined. For example, this can involve an escalation from basal-only to MDI. As another example, this can involve an escalation from use of a particular medication (e.g., glucagon-like peptide-1) to use of the medication with a basal dose. Considerations for sharing parameters (e.g., insulin sensitivity factor) between algorithms can be defined.

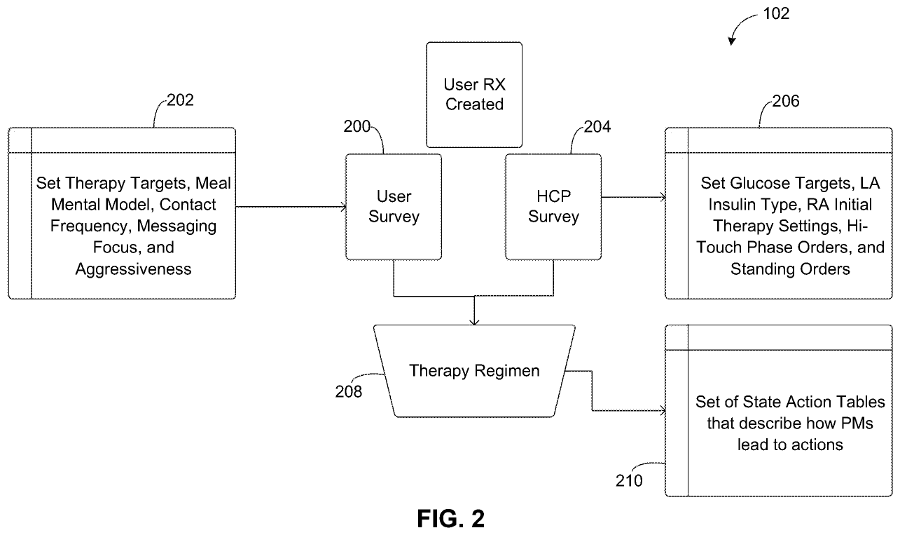

FIG. 2 shows an example of the pre-training survey stage 102 of the process 100 of FIG. 1. The pre-training survey stage 102 can include a user survey 200. For example, the user can make input to the user survey 200 by typing and/or speaking. In some implementations, the PWD is asked a number of questions to determine their thoughts about their current therapy. The answers to these questions can be used to determine a psychographic profile of the PWD. The system can determine how much the PWD is afraid of hypoglycemia and what drives their decisions about their therapy (e.g., cost, time, help from family, understanding how to treat their diabetes, etc.). This can facilitate making one or more settings 202. For example, the settings 202 can include setting therapy targets, a meal mental model, defining contact frequency, defining a messaging focus, and/or specifying an aggressiveness for communications/actions.

The pre-training survey stage 102 can include a HCP survey 204. The HCP survey 204 can ask the HCP to set therapy parameters. This can facilitate making one or more settings 206. For example, the settings 206 can include setting glucose targets, a long-acting (LA) insulin type, a rapid-acting (RA) insulin type, a RA initial therapy setting, a high-touch phase order, and/or a standing order.

The pre-training survey stage 102 can include defining a therapy regimen 208. The therapy regimen 208 can include a set of parameters that define a therapy. These can include insulin dose and mental model, and/or therapy adjustment aggressiveness, target glucose, contact frequency, method of communication, and coaching focus. This can facilitate creating one or more state action tables 210. For example, the state action tables 210 can define how performance metrics lead to actions/recommendations.

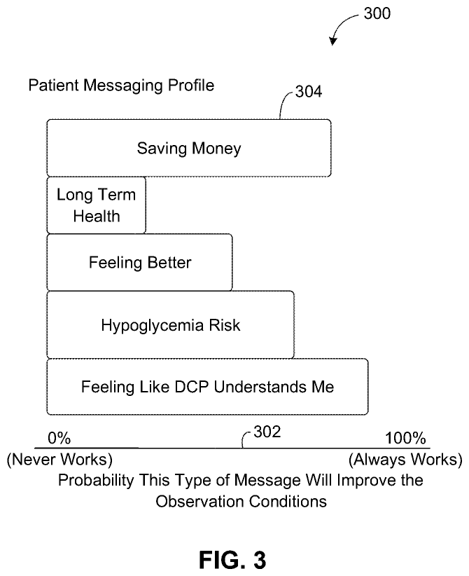

FIG. 3 shows an example of a patient messaging profile 300 that can be created in the process 100 of FIG. 1. The patient messaging profile 300 can be used in combination with one or more other examples described elsewhere herein. The patient messaging profile 300 includes a scale 302 against which profile contents can be defined. The scale 302 can specify a probability. For example, the probability can reflect the likelihood that a particular type of message will improve an observed condition. The patient messaging profile 300 can include one or more entries 304 measured against the scale 302. For example, the entries 304 can specify one or more of: saving money, long-term health, feeling better, hypoglycemia risk, and/or feeling like the diabetes care provider (DCP) understands the PWD. Other approaches can be used.

Based on the assessment reflected by the patient messaging profile 300 a recommended goal would be provided to a PWD. The PWD may be able to change the recommended goal (e.g., within an app) as their situation may shift over time. While the PWD's goal may not change the type of or approach to the clinical insights, it may alter the messaging in how the clinical insights are surfaced. A variety of different goal statements can be used. Goals can be individualized based on duration of diabetes, age/life expectancy, comorbid conditions, known cardiovascular disease or advanced microvascular complications, hypoglycemia unawareness, and individual patient considerations. Examples of goals include but are not limited to: improve my overall long term health; reduce hypoglycemia and manage related stress levels; feeling better to be more energetic and focused; learn more about my diabetes and have a more personalized therapy; or reduce overall healthcare costs. Based on the glycemic targets, the system then leverages standard guidelines (e.g., from ADA or AACE) for titration during initiation of therapy, starting with basal therapy.

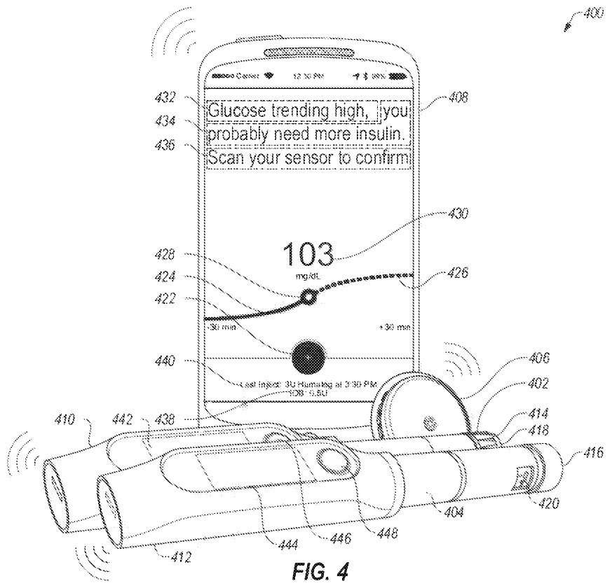

FIG. 4 shows an example of a system 400 for diabetes management. The system 400 can be used in combination with, or can include aspects of, one or more other examples described elsewhere herein. Here, the system 400 includes an insulin injection pen 402. In some implementations, the insulin injection pen 402 is considered an insulin injection pen for a certain type of insulin, such as a rapid-acting insulin pen. For example, the insulin injection pen 402 is a Humalog™ pen, a Novolog™ pen, or an Apidra™ pen. Here, the system 400 includes an insulin injection pen 404. In some implementations, the insulin injection pen 404 is considered an insulin injection pen for a certain other type of insulin having an active time that is longer than an active time for the certain type of insulin, such as a long-acting insulin pen. For example, the insulin injection pen 404 is a Lantus™ pen, a Levemir™ pen, a Toujeo™ pen, or a Tresiba™ pen. Here, the system 400 includes a glucose monitor 406 (e.g., a CGM) or another glucose sensor, and a remote user interface device 408. As shown, each insulin injection pen 402 and 404 includes a corresponding pen cap 410 and 412, respectively, which are in wireless communication with other components of the system 400. As shown, the insulin injection pens 402 and 404 can include dials 414 and 416, respectively, for a user to set a dosage to be delivered, and a respective dose indicator window 418 and 420. In some implementations, one or more of the insulin injection pens 402 and/or 404 may include dose-capture technology and/or be in wireless communication with other components of the system 400. Additional details about possible insulin pens and/or insulin pen caps are exemplified below.

Glucose monitor 406 or another glucose sensor can include any suitable sensor device and/or monitoring system capable of providing data that can be used to estimate one or more blood glucose values. As shown, glucose monitor 406 or another glucose sensor can be a sensor configured to transmit blood glucose data wirelessly. For example, the glucose monitor 406 or another glucose sensor can include an optical communication device, an infrared communication device, a wireless communication device (such as an antenna), and/or chipset (such as a Bluetooth device (e.g., Bluetooth Low Energy (BLE), Classic Bluetooth, etc.), an NFC device, an 802.6 device (e.g., Metropolitan Area Network (MAN), a Zigbee device, etc.), a WiFi device, a WiMax device, a Long Term Evolution (LTE) device, cellular communication facilities, etc.), and/or the like. The glucose monitor 406 or another glucose sensor may exchange data with a network and/or any other devices or systems described in the present disclosure. In some cases, the glucose monitor 406 or another glucose sensor can be interrogated with an NFC device by the user moving one or more components of the system 400 near the glucose monitor 406 or another glucose sensor to power the glucose monitor 406 or another glucose sensor, and/or to transmit blood glucose data from the glucose monitor 406 or another glucose sensor, to other components of system 400. For example, the pen cap 410 and/or 412 can exchange data with (e.g., obtain glucose values from) the glucose monitor 406 or another glucose sensor by being brought in close proximity thereof.

As shown, remote user interface device 408 is a mobile electronic device (e.g., a smartphone) in some examples. In some implementations, any suitable remote user interface device can be used, including, but not limited to, a computer tablet, a smartphone, a wearable computing device, a smartwatch, a fitness tracker, a laptop computer, a desktop computer, a smart insulin pen (e.g., the pen caps 410 and/or 412), and/or other appropriate computing devices. As shown in the exemplary user interface of the exemplary mobile app running on the depicted smartphone, the user interface can include a bolus calculator button 422 and optionally other buttons for the user to enter data or to request recommendations. The exemplary user interface can also or instead include a display of blood glucose data (e.g., past, present, and/or predicted). As shown, the user interface includes a graph 424 of historical data (e.g., from the previous 30 minutes), a continuation 426 of that graph having projected data, a point indicator 428 showing the current (or most recent) estimated blood glucose value, and a display 430 of the current (or most recent) estimated blood glucose value. The user interface can also or instead include text 432 explaining the glucose data, text 434 and/or text 436 providing suggested actions. For example, text 434 can provide insulin, carbohydrates, or other therapy suggestions. For example, text 436 can suggest that the user obtain blood glucose data. In some cases, the user interface can permit the user to click on the glucose data or otherwise navigate in the mobile app to obtain more detailed or more complete blood glucose data. In some implementations, the user interface can also or instead provide one or more insights for the benefit of the person with diabetes. For example, an insight can communicate a message along the lines of “You normally have a meal bolus between noon and 2 PM, but didn't bolus today and your blood glucose levels are rising. Did you forget to bolus?”

The user interface can depict insulin data. In some cases, the user interface can indicate an amount 438 of IOB, which may be only for a particular type of insulin (including, but not limited to, rapid-acting insulin). In some implementations, IOB (sometimes referred to as active insulin) can be defined as the amount of insulin that has been delivered and is still active in the person's body based on estimated duration of insulin action. In some cases, an IOB calculation may be for both rapid-acting and long-acting insulin. In some cases, the user interface can display information 440, including, but not limited to, the time and/or amounts of the most recent doses of rapid-acting and/or long-acting insulins. In some cases, the user interface can permit the user to click on the insulin data or otherwise navigate in the mobile app to more detailed and/or more complete insulin delivery data. In some cases, a user interface can overlay blood glucose data and insulin delivery data in any suitable format, such as a graphical display of the timing of blood glucose data vs. the timing of insulin delivery data.

In use, a user (e.g., the PWD and/or a HCP) can use the system 400 to get recommendations regarding an appropriate insulin dosage. In the case of an upcoming need to deliver long-acting insulin, the text 434 may be changed to provide a recommended long-acting insulin dosage. In some cases, a recommended dosage may appear on pen cap 410 and/or 412. In the case of the user wanting to deliver a bolus of rapid-acting insulin, the user may press bolus calculator button 422 to enter into a bolus calculator. Any suitable bolus calculator could be used in systems, methods, and devices provided herein. For example, the bolus calculator can provide a user interface for a user to enter a meal announcement as either a correction only, a small meal, a normal sized meal, or a large meal. Upon selecting the meal size, the user interface can provide a recommended bolus dosage based on a number of carbohydrates associated with the corresponding button and optionally based upon blood glucose data. Additionally, or alternatively, dose capture pen caps for the insulin injection pen 402 and/or 404 can include a user interface that permits the user to obtain a meal bolus recommendation for different types of meals (small, medium, large; breakfast, lunch, dinner; 10 grams carbs, 20 grams carbs, 45 grams carbs; etc.) and/or announce a meal size, including, but not limited to, a small meal, medium meal, or large meal. In some implementations, the text 432, 434, and/or 436 is presented elsewhere than on the user interface device 408. For example, presentation can be done on the pen cap 410 and/or 412.

The pen cap 410 and/or 412 can include at least one display. In some implementations, the pen cap 410 includes a display 442. In some implementations, the pen cap 412 includes a display 444. The display 442 and/or 444 can comprise any suitable type of display technology, including, but not limited to, a dynamic electronic display (e.g., a light-emitting diode display) or a static electronic display (e.g., an E-Ink display). The display 442 and/or 444 can present information to a user, including, but not limited to, any information output that is described elsewhere herein. For example, an insulin dosage suggestion and/or an alert or other communication can be presented.

The pen cap 410 and/or 412 can include at least one input control. In some implementations, the pen cap 410 includes a button 446. In some implementations, the pen cap 412 includes a button 448. The button 446 and/or 448 can comprise any suitable type of input technology, including, but not limited to, an electronic switch. The button 446 and/or 448 can trigger the corresponding pen cap 410 and/or 412 to perform one or more operations, including, but not limited to, any operation that is described elsewhere herein.

The pen cap 410 and/or 412 can record and/or convey one or more types of pen capping information. Pen capping information (e.g., information about when the pen cap is secured to and/or released from the injection pen) can include information about a current capping period (e.g., the time since the last capping), information about a duration of one or more uncappings (which may also be referred to herein as “decapping(s)”), and/or the timing (e.g., time-of-day or time elapsed since) of each uncapping and each capping. For example, capping information can include data reflecting when the pen cap was placed onto the insulin injection pen, data reflecting when the pen cap was removed from the insulin injection pen, or both. In some embodiments, pen capping information may be presented to a user on a display of the pen cap. In some embodiments, pen capping information may be presented by a speaker in the pen cap. For example, in some embodiments, a pen cap may provide a timer clock that counts up from the last time the pen cap was secured to the injection pen. In some embodiments, a pen cap accessory can wirelessly communicate pen capping information to a remote computing device (e.g., the user interface device 408 and/or a smartphone, tablet, etc.). In some embodiments, one or more accessories or smart delivery devices can detect other events associated with medication delivery actions and use that information in ways that pen capping information is described herein. For example, in some cases an injection pen accessory may be secured to an injection pen such that it can detect the mechanical movement of the dosing mechanism to determine a time of a dose of medication (and optionally but not necessarily an amount of medication delivered at that time).

Pen capping information may be stored, displayed, and/or analyzed in combination with glucose data to determine user behaviors, such as, for example, whether the person is appropriately dosing insulin for meals and/or to correct elevated blood glucose levels. In some embodiments, pen capping information may be presented on a graphical representation of blood glucose data for the user and presented to a user and/or to a healthcare professional. In some embodiments, blood glucose data from a period of time after each capping event may be evaluated to determine whether the user appropriately dosed insulin for that capping event, e.g., appropriate dose, under dose, or over dose.

In some embodiments, a pen capping event may be disregarded where other information indicates that a dose was not provided. For example, where no change in the dosage selection of the insulin pen (e.g., a dial) was detected, the event may be disregarded. In some embodiments, a pen uncapping and recapping event may be disregarded if the total uncapping time is less than a first threshold (e.g., 4-6 seconds). For example, the threshold may be determined by setting it at an amount of time too short to permit for an injection, but long enough to allow a user to check the end of the pen to see if there is insulin remaining or if there is a needle attached to the pen. In some cases, the total decapping time (the time between an uncapping event and the subsequent recapping) for a decapping event may be analyzed in combination with blood glucose data to determine if there was an injection during that decapping event. In some cases, if the total decapping time exceeds a second threshold period of time (e.g., at least 15 minutes, at least 30 minutes, etc.), blood glucose data and the methods described herein for detecting a meal timing may be used to determine an approximate time of an injection.

Some or all components of the system 400 can perform one or more operations or functions described elsewhere herein. In some implementations, the system 400 can evaluate insights based on reinforcement learning and perform one or more actions for a triggered insight. The system 400 can determine insulin use by detecting removal and/or replacement of the pen cap 410 and/or 412. The system 400 can detect an amount of insulin that is injected.

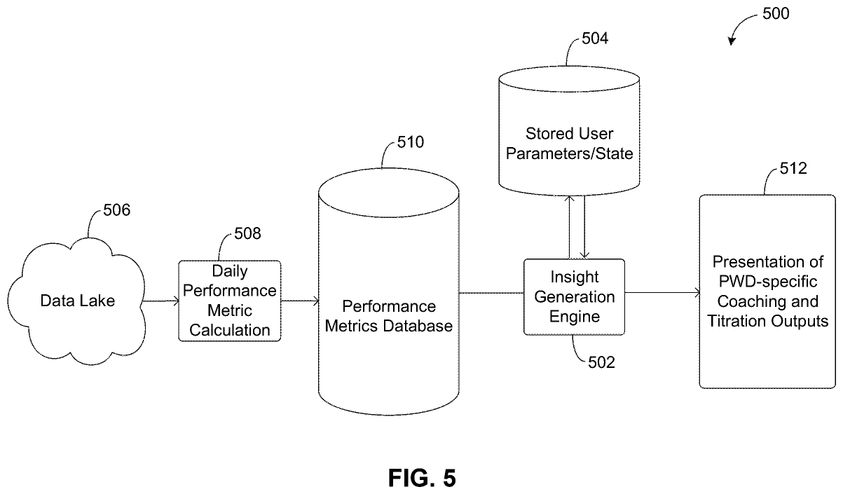

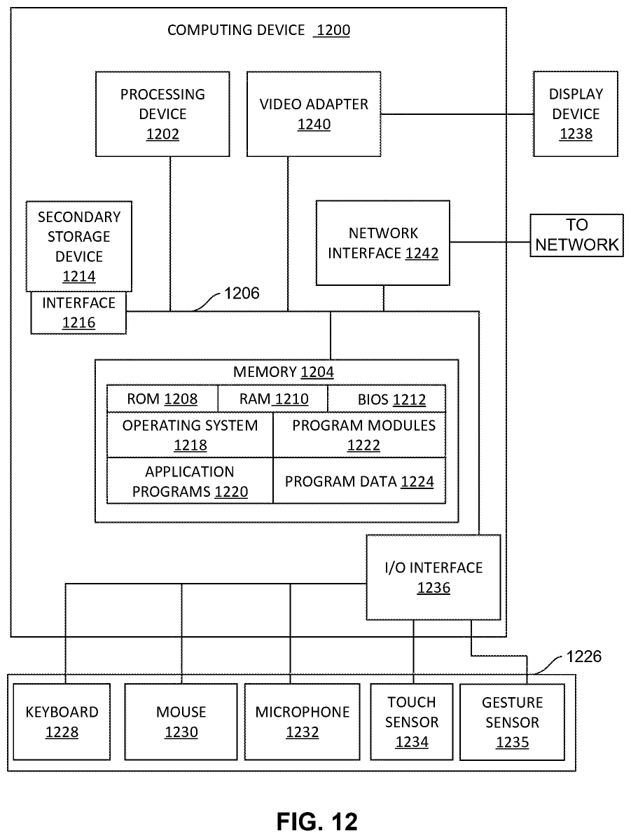

FIG. 5 shows an example of a system 500 for guiding a person with diabetes in managing their insulin therapy. The system 500 can be used in combination with, or can include aspects of, one or more other examples described elsewhere herein. The system 500 is shown as including multiple separate components. The functionality of the system 500 can be implemented using more or fewer components. The system 500 includes an insight generation engine 502 that can be implemented using components described below with reference to FIG. 12. The insight generation engine 502 evaluates multiple insights based on reinforcement learning and decides, for each of the insights, whether to generate that insight. Each insight can be associated with performing one or more actions. For example, performing an action can include providing a message to the PWD and/or automatically changing the insulin therapy. The insights can be designed so as to cover the PWD's entire journey with diabetes. The insights can involve observations from data that should be called out so that actions can be taken. Insights should be relevant enough that if appropriate actions are taken the PWD's MDI therapy can be significantly improved.

Insights can describe conditions of sub-optimal MDI therapy which should be followed by an action. The insight generation engine 502 can evaluate the insight conditions and determine which insights meet the triggering criteria. Triggered insights are followed up with actions. To evaluate if the action was successful, the system 500 can monitor the PWD during an observation period. If the conditions improved, the system 500 can give a positive reward to the insight generation engine 502 and update the parameters so the insight generation engine 502 would be more likely to take such an action in the future. If the conditions fail to improve, the system 500 can give a negative reward to the insight generation engine 502 and update the parameters so that the insight generation engine 502 would be less likely to take such an action in the future. After the observation period, the system 500 may not allow the insight to be triggered for some time, a scenario that can be referred to as a cooldown period. This ensures that the system 500 does not overwhelm the PWD with too many actions. If the PWD's state does not trigger the insight for a long period of time, the insight generation engine 502 can trigger a streak reward to celebrate their accomplishment.

An insight can have multiple parameters in a store 504 accessible to the insight generation engine 502. The parameters can include, but are not limited to: trigger conditions, such as a set of conditions based on performance metrics that indicate a coaching action should be taken, for example when fewer than a fixed amount of RA doses are taken per time period and the PWD spends less than a threshold amount of time within blood glucose level range; coaching actions, such as a set of possible actions that can be taken to help the PWD improve, for example a message or a therapy adjustment, perhaps to present a card with the PWDs postprandial glucose when the PWD takes a RA dose; observation window, for measuring how effective the actions are, so the system 500 can adjust the approach(es) over time, for example an observation window specifying how long to observe before determining whether the intervention was successful (some interventions may take longer to see in the performance metric data, so this parameter can allow the system 500 to look longer before determining if the intervention worked); cooldown period, to avoid telling the PWD over and over that they are not doing something right, a cooldown period can be defined to ensure that an insight is not triggered too frequently (for therapy adjustments, the system 500 may limit the rate of adjustment as well, such as to set a two-week cooldown period to ensure that the system 500 does not consider another therapy adjustment too soon), wherein two or more cooldown periods can be linked (e.g., triggering a specific insight might start a cooldown for multiple insights); celebration conditions, such as conditions under which the system 500 would celebrate the lack of an insight triggering over some period of time, for example by presenting the message “Congratulations, you've taken your <long acting dose brand> on time every day for the last 60 days!” The store 504 can reflect a state of the PWD with regard to insights. Because insight generation is based on knowing the observation and cooldown periods, a user state is considered before an insight is determined.

The system 500 can make use of data about, or that otherwise relates to, the PWD. The data can be furnished in any of multiple ways, such as by a data lake 506. In some implementations, the data lake 506 can be a centralized repository that stores, processes, and secure large amounts of structured, semi-structured, and/or unstructured data. For example, the data lake 506 can be provided by cloud infrastructure that collects sensor readings, smart cap removals/replacements, user inputs, and/or other information about the PWD. At least one calculation 508 can be performed in the system 500 based on information from the data lake 506. In some implementations, a performance metric calculation can be performed. The performance metric can be calculated at regular intervals, such as on a daily basis. Examples of performance metrics include, but are not limited to: number of removals or replacements of a smart cap per day for a first injection pen (e.g., with long-acting insulin); number of removals or replacements of a smart cap per day for a second injection pen (e.g., with rapid-acting insulin); time in range; or level of postprandial glucose. The performance metrics can be stored in a performance metric database 510 that is accessible to the insight generation engine 502.

The system 500 can include a component 512 presenting output from the insight generation engine 502. In some implementations, the output of the component 512 is specific to the PWD. For example, the output can include PWD-specific coaching and/or titration outputs.

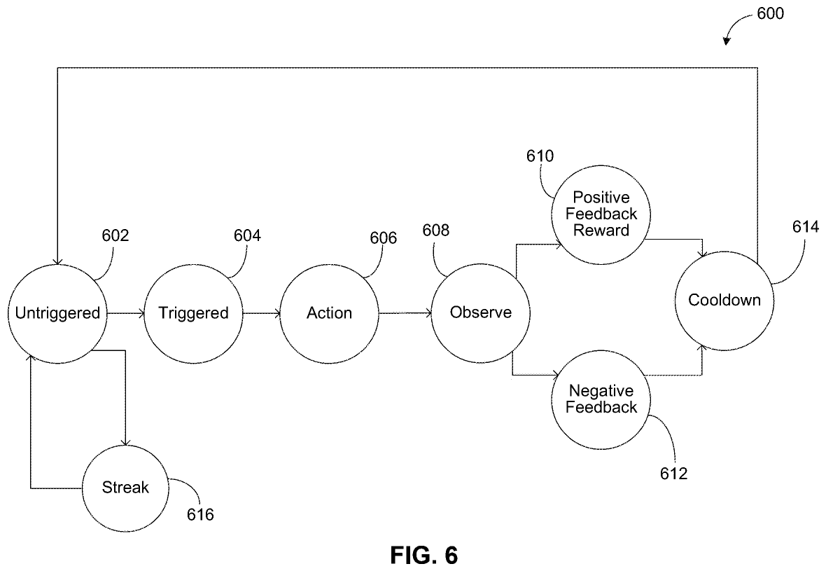

FIG. 6 shows an example of a state diagram 600 that can be used by the insight generation engine 502 of FIG. 5. The state diagram 600 can also or instead be used in combination with one or more other examples described elsewhere herein. The state diagram 600 represents different states for at least one insight, each of which states is schematically represented as a circle, with arrows indicating possible state transitions. The state diagram 600 includes an untriggered state 602. For example, this can be the default initial state of each insight. A triggered state 604 indicates that a determination to trigger the insight has been made. For example, the insight is triggered based on an evaluation of performance metrics indicating that the criteria for the insight are satisfied. An action state 606 indicates that at least one action associated with the triggered insight is performed in response to the triggering. An observe state 608 indicates that the triggered insight is under observation after being triggered. The observation period can be used for gathering information to be used for reinforcement learning. A positive feedback reward state 610 indicates that they system is given positive feedback based on the observation(s) relating to the triggered insight. A negative feedback reward state 612 indicates that they system is given negative feedback based on the observation(s) relating to the triggered insight. During the observe state 608, the positive feedback reward state 610, and the negative feedback reward state 612 the triggered insight may not be eligible to be triggered. The observe state 608 includes time after an insight is triggered where the effect of the action is observed. With some insights, the system may look for performance metrics to change to acceptable levels. With other insights, the system could use specialized machine learning methods to determine the success of the action. As an example, updates to long-acting insulin can be determined using a machine learning method that examines glucose patterns to confirm that insulin dosing has been changed.

A cooldown state 614 indicates that the triggered insight is in a cooldown period after the observe state 608. During the cooldown state 614 the triggered insight may not be eligible to be triggered. After the cooldown state 614 the insight may again assume the untriggered state 602. During the untriggered state 602, an evaluation can be made, for each insight, whether the insight has not been triggered for at least a threshold amount of time. Such a period can be considered a favorable or desirable circumstance for the PWD in that their diabetes has been managed so that this particular coaching or change in therapy have not been needed during that period. A streak state 616 indicates that the insight has not been triggered for at least a threshold amount of time, amounting to a streak of success. For example, the streak state 616 can include presenting a celebratory message to the PWD.

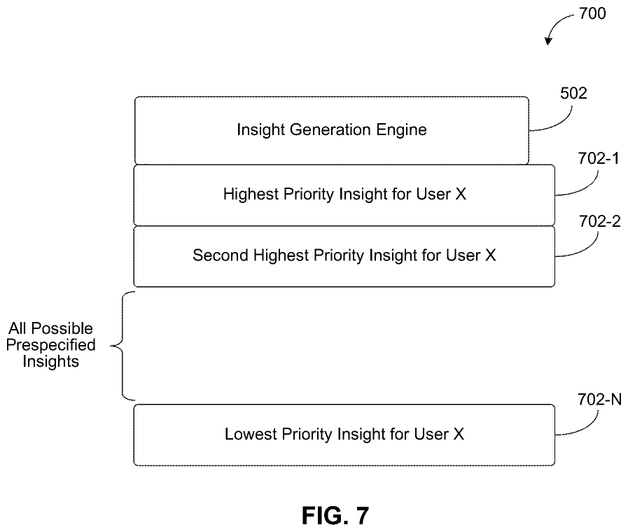

FIG. 7 schematically shows a prioritization 700 of insights that can be performed by the insight generation engine 502 of FIG. 5. The prioritization 700 can also or instead be used in combination with one or more other examples described elsewhere herein. The prioritization 700 is performed for insights 702-1 through 702-N, where N=2, 3, . . . . The PWD is here referred to as user X. For example, insights potentially affecting the life of the PWD can be given the highest priority. As another example, insights potentially affecting the health of the PWD can be given a priority less than the life-affecting insights (e.g., a second priority) and higher than at least one other insight. As another example, insights relating to the improvement or optimization of the PWD's diabetes care that do not affecting the life or health of the PWD can be given a priority less than that of the health-affecting insights. Based on the prioritization 700, the insight 702-1 can be deemed the highest priority insight. An insight 702-2 can be deemed the second highest priority insight. The insight 702-N can be deemed the lowest priority insight. As such, multiple insights can be ranked in the prioritization 700 and evaluated in that order.

Competition between insights can occur. An insight competition can be a process by which the system (e.g., the insight generation engine 502 of FIG. 5) determines the most relevant insights. That is, the insights may have a natural precedence compared to each other (e.g., a described above). Nevertheless, multiple positive actions (e.g., celebrations and streaks) and negative actions (coaching or therapy adjustment needed) can be possible given the current state of the PWD and the data. The insight competition can choose the correct insight(s) to present to the PWD. This can ensure a balance in the number and type of insights that are presented at one time.

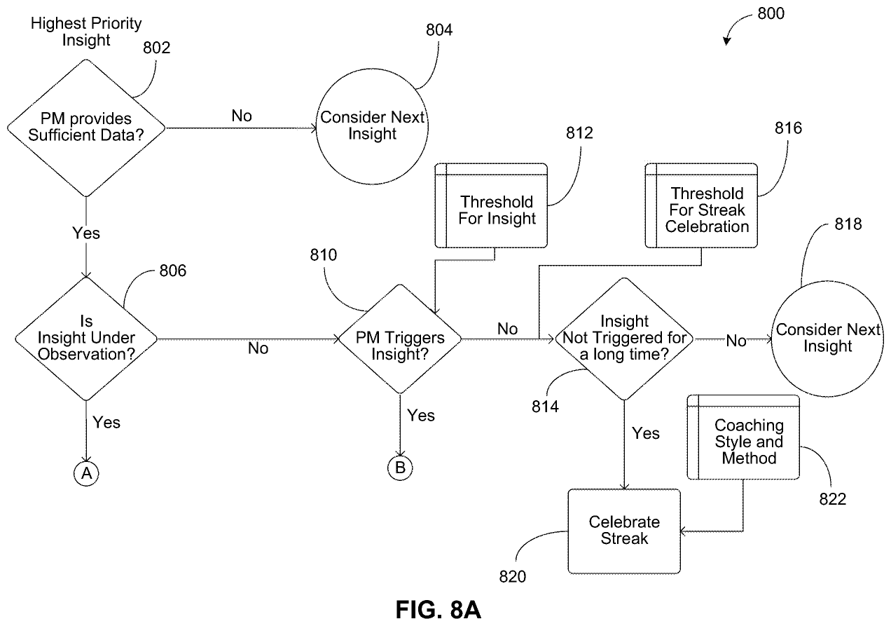

FIGS. 8A-8B show an example of a process 800 that can be performed by the insight generation engine of FIG. 5. The process 800 can also or instead be used in one or more other examples described elsewhere herein. More or fewer operations than shown can be performed. Two or more operations can be performed in a different order unless otherwise indicated. The process 800 can be performed repeatedly to evaluate respective ones of multiple insights in order of their prioritization. Here, the process 800 is labeled “highest priority insight” to indicate that the insight currently being evaluated is the highest-priority insight that has not already been evaluated in a previous iteration of the process 800.

In operation 802, a determination is made whether the performance metrics (e.g., in the performance metric database 510 of FIG. 5) provide sufficient data. The sufficiency criterion can vary depending on the insight. For example, if the system has not seen a sufficient number of lunch events for the PWD over the past period of time to make a firm assessment of what occurs at lunchtime for the PWD, an insight regarding lunch can be skipped. If the outcome of the operation 802 is no, the process at operation 804 can proceed to a next insight in the order of priority. For at least one insight, the dearth or lack of performance metrics can satisfy the condition for triggering the insight. For example, if the system has too little data, a coaching message can be generated that encourages the PWD to use the system more so that data about their diabetes management is obtained.

If the outcome of the operation 802 is yes, then in operation 806 a determination can be made whether the current insight is under observation (e.g., the observe state 608 of FIG. 6). If the insight is not under observation, then the insight is eligible for trigger evaluation (but may not be triggered depending on circumstances). Accordingly, if the outcome of the operation 806 is no, a determination can be made in an operation 810 of whether the performance metrics trigger the insight. The determination can take into account settings 812, such as one or more thresholds for the insight.

If the insight is not triggered by the current state of the PWD, then it is possible that the PWD is on a streak with regard to not triggering this particular insight. If the outcome of the operation 810 is no, then in operation 814 a determination can be made whether the insight has not been triggered for a specified length of time. The determination can take into account settings 816, such as one or more thresholds for streak celebration. If the outcome of the operation 814 is no, then in operation 818 the process 800 can proceed to a next insight in the order of priority. If the outcome of the operation 814 is yes, then in operation 820 a streak celebration can be performed. The streak celebration can be performed based on settings 822, such as one or more styles or methods for coaching.

If the outcome of the operation 810 is yes, then in operation 824 a determination can be made whether the insight is currently in a cooldown period (e.g., the cooldown state 614 of FIG. 6). The determination can take into account settings 826, such as one or more thresholds for the insight. If the outcome of the operation 824 is yes, then at operation 828 the process 800 can proceed to a next insight in the order of priority. If the outcome of the operation 824 is no, then at operation 830 the process 800 can perform one or more actions associated with the triggered insight. For example, the action can involve coaching and/or therapy adjustment. The action can take into account settings 832, such as one or more styles or methods for coaching. After performing the action(s), observation for the insight can begin at an operation 834 (and the process 800 can proceed to evaluate a next insight in the order of priority).

Returning now to the operation 806, if the outcome of this determination is yes, then in operation 836 a determination can be made whether one or more improvement criteria have been satisfied. The determination can take into account settings 838, such as one or more improvement thresholds for the insight. If the outcome of the operation 836 is no, then in operation 840 a determination can be made whether the end of the observation window has been reached. The determination can take into account settings 842, such as a definition of the observation window for the insight. If the outcome of the operation 840 is no, then in operation 844 the observation regarding the insight can continue (and the process 800 can proceed to evaluate a next insight in the order of priority). If the outcome of the operation 840 is yes, then in operation 846 the parameters of the reinforcement learning can be updated with negative feedback regarding the action(s) previously performed for this insight (and the process 800 can proceed to evaluate a next insight in the order of priority).

If the outcome of the operation 836 is yes, then in operation 848 an improvement celebration can be performed. The improvement celebration can take into account settings 850, such as one or more styles or methods for coaching. After the operation 848, the parameters of the reinforcement learning can be updated in an operation 852 with positive feedback regarding the action(s) previously performed for this insight (and the process 800 can proceed to evaluate a next insight in the order of priority).

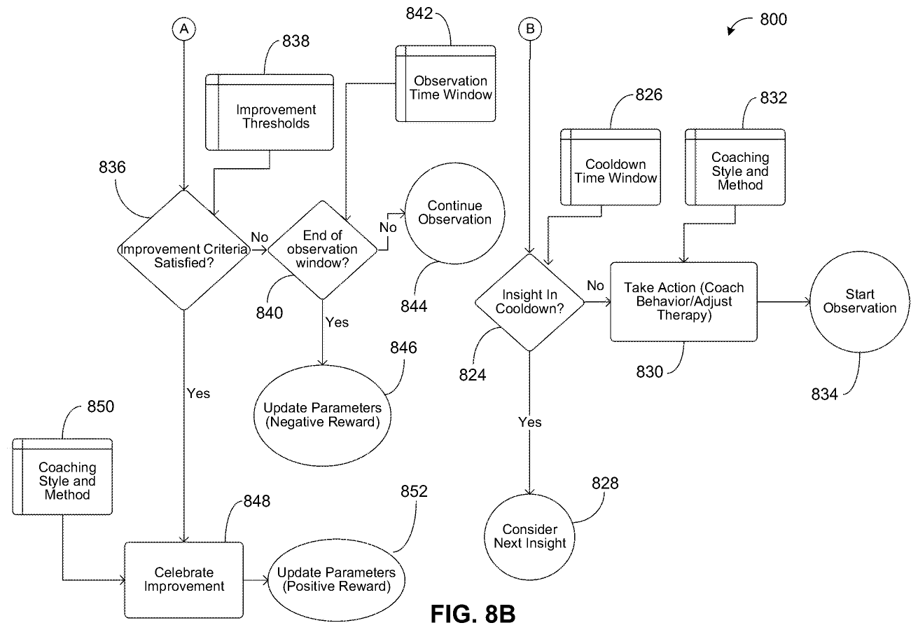

FIG. 9 shows an example of multimodal insight triggering 900. The multimodal insight triggering 900 can be used in combination with one or more other examples described elsewhere herein. The multimodal insight triggering 900 here relates to an insight 902. For example, the insight 902 can relate to high morning glucose and whether to increase a dose of long-acting insulin. The insight 902 can be triggered based on two or more criteria 904 being satisfied. Each of the criteria can specify one or more circumstances that the performance metrics can reflect. In some implementations, N number of the criteria 904 is used, where N=1, 2, 3, . . . .

The multimodal insight triggering 900 can be multifaceted and can span more than one type of performance metrics, including but not limited to behavioral metrics, physiological metrics, and/or therapy metrics. Examples of the criteria 904 include, but are not limited to: that the PWD has more than a predefined number of days per week with a long-acting dose (e.g., a behavioral metric); that the PWD has less than a predefined amount of time below range (e.g., a physiological metric); that the PWD has more than a predefined amount of coverage in glucose data (e.g., a behavioral metric); that the PWD has treated overnight low glucose less than a predefined number of times per month; that the PWD has less than a predefined amount of time with overnight low glucose; that the PWD has less than a predefined level of median minimum morning glucose; or that the PWD's daily ratio between doses of long-acting and rapid-acting insulin is less than a predefined value (e.g., a therapy metric). Accordingly, the operation 810 can take into account one or more of the criteria 904 in the determination whether the insight 902 is triggered.

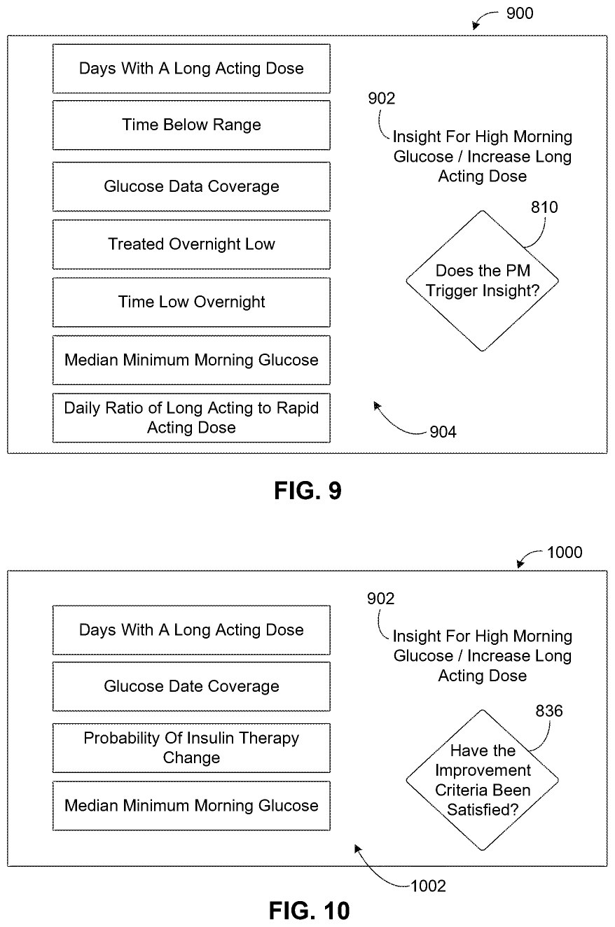

FIG. 10 shows an example of multimodal insight satisfaction evaluation 1000. The multimodal insight satisfaction evaluation 1000 can be used in combination with one or more other examples described elsewhere herein. The multimodal insight satisfaction evaluation 1000 here relates to the insight 902 of the example described above. The insight 902 can have two or more criteria 1002 for being satisfied. Each of the criteria 1002 can specify one or more circumstances that the performance metrics can reflect. In some implementations, M number of the criteria 1002 is used, where M=1, 2, 3, . . . . The number M of satisfaction criteria can be the same number as the number N of triggering criteria, or can be a higher or lower number.

The multimodal insight satisfaction evaluation 1000 can be multifaceted and can span more than one type of performance metrics, including but not limited to behavioral metrics, physiological metrics, and therapy metrics. Examples of the criteria 1002 include, but are not limited to: that the PWD has more than a predefined number of days per week with a long-acting dose (e.g., a behavioral metric); that the PWD has more than a predefined amount of coverage in glucose data (e.g., a behavioral metric); that the PWD has a probability of insulin therapy change greater than a predefined number; or that the PWD has less than a predefined level of median minimum morning glucose. Accordingly, the operation 836 can take into account one or more of the criteria 1002 in the determination whether the improvement criteria of the insight 902 are satisfied. That is, satisfaction (or not) of the criteria 1002 determines whether the reinforcement learning generates positive (or negative) feedback for the action(s) taken with regard to the insight 902.

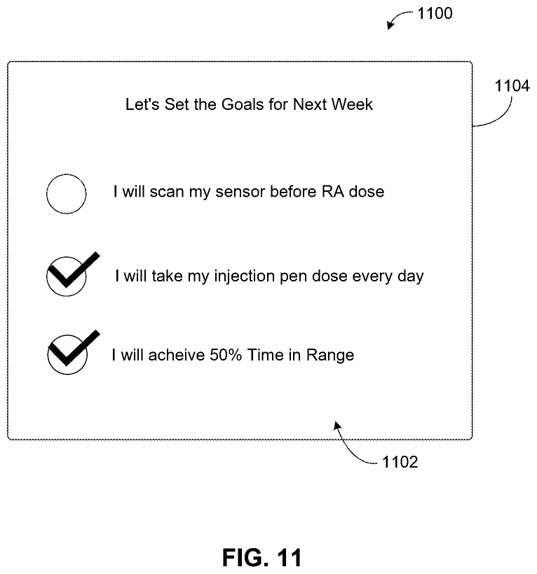

FIG. 11 shows an example 1100 of actions 1102 relating to an insight. The actions 1102 can be used in combination with one or more other examples described elsewhere herein. The actions 1102 can be presented on a display device 1104 (e.g., in an app on a mobile electronic device). The PWD can opt in to (or opt out of) any of the actions 1102. For example, an opt-in input and/or an opt-out input can be received. The actions 1102 can be presented to encourage the PWD to set goals for managing their diabetes therapy.

FIG. 12 illustrates an example architecture of a computing device 1200 that can be used to implement aspects of the present disclosure, including any of the systems, apparatuses, and/or techniques described herein, or any other systems, apparatuses, and/or techniques that may be utilized in the various possible embodiments.

The computing device illustrated in FIG. 12 can be used to execute the operating system, application programs, and/or software modules (including the software engines) described herein. The computing device can make use of at least one non-transitory computer-readable storage medium.

The computing device 1200 includes, in some embodiments, at least one processing device 1202 (e.g., a processor), such as a central processing unit (CPU). A variety of processing devices are available from a variety of manufacturers, for example, Intel or Advanced Micro Devices. In this example, the computing device 1200 also includes a system memory 1204, and a system bus 1206 that couples various system components including the system memory 1204 to the processing device 1202. The system bus 1206 is one of any number of types of bus structures that can be used, including, but not limited to, a memory bus, or memory controller; a peripheral bus; and a local bus using any of a variety of bus architectures.

Examples of computing devices that can be implemented using the computing device 1200 include a desktop computer, a laptop computer, a tablet computer, a mobile computing device (such as a smart phone, a touchpad mobile digital device, or other mobile devices), or other devices configured to process digital instructions.

The system memory 1204 includes read only memory 1208 and random access memory 1210. A basic input/output system 1212 containing the basic routines that act to transfer information within computing device 1200, such as during start up, can be stored in the read only memory 1208.

The computing device 1200 also includes a secondary storage device 1214 in some embodiments, such as a hard disk drive, for storing digital data. The secondary storage device 1214 is connected to the system bus 1206 by a secondary storage interface 1216. The secondary storage device 1214 and its associated computer readable media provide nonvolatile and non-transitory storage of computer readable instructions (including application programs and program modules), data structures, and other data for the computing device 1200.

Although the example environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media. For example, a computer program product can be tangibly embodied in a non-transitory storage medium. Additionally, such computer readable storage media can include local storage or cloud-based storage.

A number of program modules can be stored in secondary storage device 1214 and/or system memory 1204, including an operating system 1218, one or more application programs 1220, other program modules 1222 (such as the software engines described herein), and program data 1224. The computing device 1200 can utilize any suitable operating system, such as Microsoft Windows™, Google Chrome™ OS, Apple OS, Unix, or Linux and variants and any other operating system suitable for a computing device. Other examples can include Microsoft, Google, or Apple operating systems, or any other suitable operating system used in tablet computing devices.

In some embodiments, a user provides inputs to the computing device 1200 through one or more input devices 1226. Examples of input devices 1226 include a keyboard 1228, mouse 1230, microphone 1232 (e.g., for voice and/or other audio input), touch sensor 1234 (such as a touchpad or touch sensitive display), and gesture sensor 1235 (e.g., for gestural input). In some implementations, the input device(s) 1226 provide detection based on presence, proximity, and/or motion. In some implementations, a user may walk into their home, and this may trigger an input into a processing device. For example, the input device(s) 1226 may then facilitate an automated experience for the user. Other embodiments include other input devices 1226. The input devices can be connected to the processing device 1202 through an input/output interface 1236 that is coupled to the system bus 1206. These input devices 1226 can be connected by any number of input/output interfaces, such as a parallel port, serial port, game port, or a universal serial bus. Wireless communication between input devices 1226 and the input/output interface 1236 is possible as well, and includes infrared, BLUETOOTH® wireless technology, 802.11a/b/g/n, cellular, ultra-wideband (UWB), ZigBee, or other radio frequency communication systems in some possible embodiments, to name just a few examples.

In this example embodiment, a display device 1238, such as a monitor, liquid crystal display device, light-emitting diode display device, projector, or touch sensitive display device, is also connected to the system bus 1206 via an interface, such as a video adapter 1240. In addition to the display device 1238, the computing device 1200 can include various other peripheral devices (not shown), such as speakers or a printer.

The computing device 1200 can be connected to one or more networks through a network interface 1242. The network interface 1242 can provide for wired and/or wireless communication. In some implementations, the network interface 1242 can include one or more antennas for transmitting and/or receiving wireless signals. When used in a local area networking environment or a wide area networking environment (such as the Internet), the network interface 1242 can include an Ethernet interface. Other possible embodiments use other communication devices. For example, some embodiments of the computing device 1200 include a modem for communicating across the network.

The computing device 1200 can include at least some form of computer readable media. Computer readable media includes any available media that can be accessed by the computing device 1200. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 1200.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 12 is also an example of programmable electronics, which may include one or more such computing devices, and when multiple computing devices are included, such computing devices can be coupled together with a suitable data communication network so as to collectively perform the various functions, methods, or operations disclosed herein.

### Exemplary Therapy Management System

As discussed above, a permanent therapy that provides constant glycemic control is needed in order to constantly maintain the level of glucose within acceptable limits. Such glycemic control can be achieved by regularly supplying external drugs to the body of PWD to thereby reduce the elevated levels of glucose. An external biologically effective drug (e.g., insulin or its analog) is commonly administered by means of daily injections. In some cases, multiple daily injections of a mixture of RA insulin and LA insulin are administered via a reusable transdermal liquid dosing device.

Currently, methods to monitor A1C levels are limited to every 3-6 months depending on a variety of factors. This method of measuring patient progress and follow-up limited to quarterly or semi-annual visits can contribute to marked therapeutic inertia throughout the care of PWD, resulting in suboptimal outcomes and a “treat-to-failure” approach to diabetes therapy. Further, limited human resources continue to perpetuate the delay in advancing therapy according to standards of care.

Aspects of therapy management apparatuses, systems, and methods as discussed below can regularly maintain one or more glucose metrics of a user at a target level or in a target range, provide frequent therapy intervention and adjustment (e.g., on a daily or weekly basis rather than every 3-6 months), decrease a duration of intervention, and increase user adherence, outcomes, and satisfaction. System components can include, but are not limited to, glucose meters or monitors, insulin pumps, injection pens, smart caps on injection pens, an insulin-therapy software application (app), and/or cloud infrastructure.

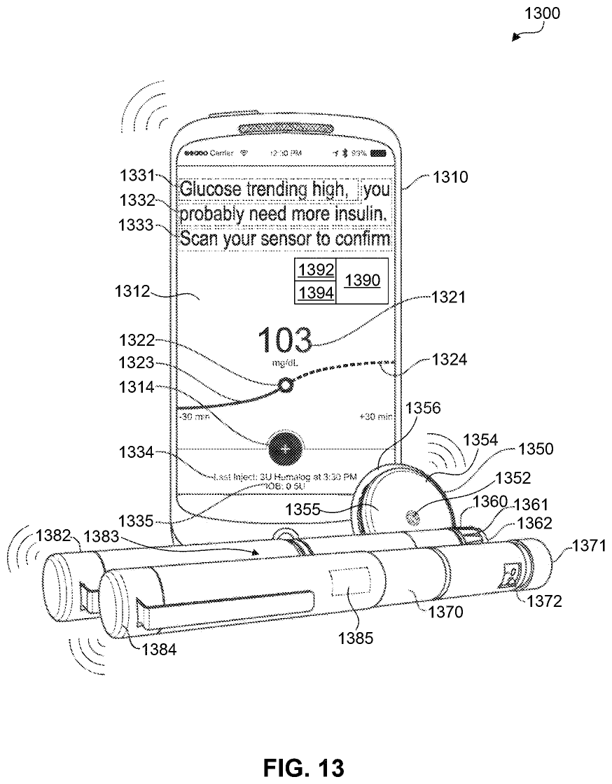

FIG. 13 illustrates therapy management system 1300, according to various exemplary aspects. Therapy management system 1300 can be configured to provide therapy escalation pathways to manage diabetes and maintain one or more glucose metrics at or within a range of one or more targets. Therapy management system 1300 can be further configured to regularly monitor one or more metrics (e.g., an A1C level) of a user. Therapy management system 1300 can be further configured to provide frequent therapy intervention and adjustment of therapy (e.g., on a daily or weekly basis rather than every 3-6 months). Therapy management system 1300 can be further configured to decrease a duration of intervention. Therapy management system 1300 can be further configured to increase user adherence, user outcomes, and user satisfaction. Therapy management system 1300 can be further configured to tailor a treatment plan to the user (e.g., based on the user's needs and values). Therapy management system 1300 can be further configured to select potential treatment plans (e.g., dosing regimen) based on past successful interventions with the user or a population (e.g., historical treatments similar to the user). Therapy management system 1300 can be further configured to provide a user and/or a medical professional with more information, more frequently, such that more options for therapy pathways are available. Therapy management system 1300 can be further configured to continuously and/or periodically monitor one or more user metrics (e.g., mean glucose level, weight, BMI, nausea, etc.) such that interventions to therapy can be timely and small. Therapy management system 1300 can be further configured to continuously monitor and improve user adherence via coaching and direct feedback (e.g., notifications, warnings, recommendations, requests for user action, etc.), such that lack of adherence and lack of therapeutic effect of the treatment can be addressed separately.

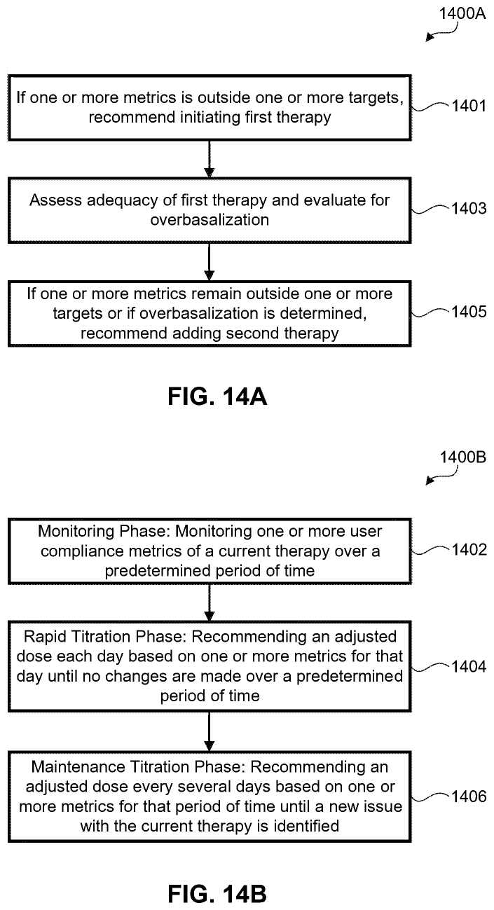

Although therapy management system 1300 is shown in FIG. 13 as a stand-alone apparatus and/or system, the aspects of this disclosure can be used with other apparatuses, systems, and/or methods, such as, but not limited to, elements in FIGS. 14A-14C, 15-18, 19A-19C, and 20-23, e.g., control diagram 1400A, therapy assessment 1400B, control diagram 1400C, therapy assessment 1430, one or more dosing regimen 1410, 1440, 1450, 1460, 1480, flow diagram 2000, flow diagram 2100, flow diagram 2200, and/or computing device 2300.

As shown in FIG. 13, therapy management system 1300 can include remote device 1310, on body unit (OBU) 1350, one or more medication delivery devices, such as a first injection pen 1360 and a second injection pen 1370, first smart cap 1382, second smart cap 1384, and/or software application 1390. In some aspects, therapy management system 1300 can include one or more memories (e.g., memory 1394) and at least one processor (e.g., processor 1392) each coupled to at least one of the memories and configured to execute a software application (app) (e.g., software application 1390) configured to monitor one or more user metrics and implement one or more therapy pathways for a user (e.g., one or more dosing regimen). In some aspects, therapy management system 1300 can include a medication delivery device (e.g., injection pen, insulin pump, etc.) in communication with software application 1390 and configured to monitor and deliver one or more medications (e.g., insulin) to the user. In some aspects, therapy management system 1300 can include a body weight scale in communication with software application 1390 and configured to measure a body weight of the user.

In some aspects, therapy management system 1300 can be configured to execute one or more therapy escalation pathways. In some aspects, therapy management system 1300 can receive glucose data of a user from a CGM, calculate one or more metrics, a processor can compare the one or more metrics to one or more thresholds stored in memory that are pre-set or user-adjustable, and output a recommendation to the user for an adjusted dose and/or a new therapy to a display of a computing device. In some aspects, therapy management system 1300 can determine if the user actually takes the recommended dose and/or new therapy, determine one or more new metrics after the user takes the recommended dose, and recommend an adjusted (titrated) dose based on the glucose data. In some aspects, therapy management system 1300 can implement a titration phase in which therapy management system 1300 requires a user to actively administer a recommended dose, reviews one or more user metrics after the user takes the medication, and then recommends a new dose based on glucose data of the user.

Remote device 1310 can be configured to receive or retrieve sensor data from analyte sensor 1352. As shown in FIG. 13, remote device 1310 can include a display 1312. Display 1312 may be a touch screen display for receiving input. Remote device 1310 may include one or more input devices for receiving input, such as one or more buttons, keys, or the like. Display 1312 may present various information to the user, such as glucose data, medication data, alerts, notifications and recommendations, among others, such as to request information from the user. The remote device 1310 may include a bolus calculator accessible via a bolus calculator button 1314. The display may show an analyte level 1321, point indicator 1322, historical analyte data 1323, trend display 1324, one or more notifications or recommendations, such as a first message 1331, second message 1332, third message 1333, first information display 1334, and second information display 1335. In some aspects, remote device 1310 can include processor 1392 coupled to memory 1394 and configured to execute software application 1390. In some aspects, remote device 1310 can include a mobile application (e.g., software application 1390) configured to perform one or more operations of therapy management system 1300 (e.g., control diagram 1400A, therapy assessment 1400B, control diagram 1400C).

As shown in FIG. 13, remote device 1310 can be in communication with OBU 1350, such as by one or more wireless communication protocols (e.g., NFC, BLE, etc.). Remote device 1310 can be in communication with one or more medication delivery devices or dose detection devices, such as a first injection pen 1360, second injection pen 1370, first smart cap 1382, second smart cap 1384, and/or software application 1390. In some aspects, remote device 1310 can include a handheld computer (e.g., smartphone, cell phone, mobile phone, PDA, smart watch, etc.), personal computer, laptop computer, a dedicated handheld device associated with the analyte sensor or medication delivery device, or any other portable communication device.

As shown in FIG. 13, in the exemplary display 1312 of the exemplary mobile app (e.g., software application 1390) running on the depicted smartphone, display 1312 can include a bolus calculator button 1314 and, optionally, one or more other buttons or inputs for a user to enter data or respond to recommendations and/or requests. In some aspects, display 1312 can include a numerical and/or a graphical display of glucose data (e.g., past, present, and/or predicted). For example, as shown in FIG. 13, display 1312 can include glucose level 1321 showing the current (or most recent) numerical estimated glucose level, point indicator 1322 graphically indicating the current (or most recent) estimated glucose value as part of an overall graph, historical analyte data 1323 showing a graph of historical glucose data (e.g., from the previous 30 minutes, etc.), and trend display 1324 showing a predicted trend or projected continuation of historical glucose data 1323.

In some aspects, display 1312 can include one or more textual messages for a user regarding the current therapy. For example, as shown in FIG. 13, display 1312 can include first message 1331 (e.g., notification), second message 1332 (e.g., recommendation), and third message 1333 (e.g., request for user action). In some aspects, first message 1331 can include a notification and/or a warning to the user, for example, explaining one or more characteristics of the glucose data (e.g., “Glucose trending high”). In some aspects, second message 1332 can include a recommendation for therapy to the user, for example, recommending one or more treatments or therapy (e.g., “You probably need more insulin”). In some aspects, second message 1332 can provide insulin, carbohydrates, and/or other therapy recommendations. In some aspects, third message 1333 can include a request for user action, for example, requesting the user to respond or perform a certain action (e.g., “Take 15 g of carbs”).

In some aspects, display 1312 can allow the user to click on the glucose data (e.g., glucose level 1321) or otherwise navigate in the mobile app (e.g., software application 1390) to obtain additional or more detailed or more complete glucose data. In some aspects, display 1312 can provide one or more insights (e.g., communication, notification, recommendation, request, etc.) for the benefit of the user (e.g., PWD). For example, an insight can communicate a tailored message to the user related to the therapy: “You normally have a meal bolus between noon and 2 PM, but did not bolus today and your glucose levels are currently rising. Did you forget to bolus?”

In some aspects, display 1312 can depict insulin data and/or dosing data. In some aspects, display 1312 can include first information display 1334 configured to display information of one or more dosing regimen, including, but not limited to, the time and/or amounts of the most recent doses of RA and/or LA insulins. In some aspects, display 1312 can include second information display 1335 configured to display information regarding one or more insulin-on-board (IOB), including, but not limited to, an amount of IOB for a particular type of insulin or insulin analog (e.g., RA, LA, etc.). In some aspects, IOB (e.g., active insulin) can be defined as an amount of insulin that has been delivered and is still active in the user's body based on an estimated duration of insulin action. In some aspects, an IOB calculation may be for both RA and LA insulin. In some aspects, display 1312 can allow the user to click on the insulin data (e.g., first information display 1334) or otherwise navigate in the mobile app (e.g., software application 1390) to obtain additional or more detailed or more complete insulin delivery data. In some aspects, display 1312 can overlay glucose data and insulin delivery data in any suitable format, for example, as a graphical display of the timing of glucose data versus the timing of insulin delivery data.

In some aspects, in use, a user (e.g., PWD and/or medical professional) can utilize therapy management system 1300 to receive recommendations regarding an appropriate insulin dosage and therapy. For example, in the case of an upcoming need to deliver LA insulin, second message 1332 can provide a recommendation for a specific LA insulin dosage. In some aspects, a recommended dosage from therapy management system 1300 (e.g., software application 1390) may appear on a display of first smart cap 1382 and/or second smart cap 1384, for example, first display 1383 and/or second display 1385, respectively.

In some aspects, the user may use bolus calculator button 1314 to enter into a bolus calculator, for example, to calculate and deliver a bolus of RA insulin. In some aspects, bolus calculator button 1314 can include any suitable bolus calculator, for example, the bolus calculator can provide a user interface for a user to enter a meal announcement as either a correction only, a small meal, a normal sized meal, or a large meal. Upon selecting a meal size or meal type (e.g., breakfast, lunch, dinner), the user interface can provide a recommended bolus dosage, for example, based on a number of carbohydrates associated with the corresponding meal size and/or meal type and, optionally, based upon glucose data.

OBU 1350 can be configured to measure and communicate sensor data of one or more analytes (e.g., glucose) of a user. OBU 1350 can be further configured to communicate data (e.g., sensor data) from analyte sensor 1352 to one or more components of therapy management system 1300 (e.g., remote device 1310, first injection pen 1360, second injection pen 1370, first smart cap 1382, second smart cap 1384, software application 1390, etc.). In some aspects, OBU 1350 can exchange data with a network and/or a remote server. As shown in FIG. 13, OBU 1350 can include analyte sensor 1352, on body electronics 1354, on body housing 1355, and/or adhesive layer 1356.

Analyte sensor 1352 can be configured to measure an analyte (e.g., glucose) of a user. Analyte sensor 1352 can be configured to measure one or more analytes (e.g., glucose, ketones) of a user. Analyte sensor 1352 can be further configured to continuously measure (e.g., in vivo) in real-time a concentration of one or more analytes of a user. In some aspects, a portion of analyte sensor 1352 (e.g., distal portion) can be positioned in vivo through a skin surface of a patient (e.g., transcutaneously) and in fluid contact with bodily fluids (e.g., interstitial fluid, etc.) of the patient. In some aspects, analyte sensor 1352 can be insertable into a body of a patient (e.g., vein, artery, skin, etc.) containing an analyte. In some aspects, analyte sensor 1352 can include CGM to continuously and automatically track glucose levels. In some aspects, analyte sensor 1352 can measure and retrieve analyte levels in real-time (e.g., about 1-60 seconds) for continuous analyte (glucose) monitoring.

In some aspects, analyte sensor 1352 can measure glucose. In some aspects, analyte sensor 1352 can measure lactate. In some aspects, analyte sensor 1352 can measure ketones. In some aspects, analyte sensor 1352 can measure one or more analytes. For example, analyte sensor 1352 can measure glucose and ketones. For example, analyte sensor 1352 can measure one or more analytes (e.g., glucose, ketones, lactate, lactic acid, oxygen, hemoglobin A1C, lactone, lactose, galactose, vitamin C, glucoronate, glycogen, mannose, phosphate, bisphosphate, fructose, glyceraldehyde, glycerol, triglycerides, sorbitol, phosphoglucono, phosphogluconate, xylulose, ribose, bile, cysteine, serine, homoserine, pyruvate, phenylpyruvate, glutamate, glycine, taurine, threonine, methionine, ethanol, acetone, acetate, oxaloacetate, alanine, phenylalanine, aspartate, asparagine, alcohol, cholesterol, vitamin D, progesterone, testosterone, estrogen, squalene, insulin, hydroxybutyrate, leucine, isoleucine, malonyl, malonate, glucagon, epinephrine, norepinephrine, palmitate, lysine, eicosanoids, melanin, dopamine, tyrosine, tryptophan, niacin, melatonin, serotonin, citrate, isocitrate, valine, porphyrins, histidine, urocanate, histamine, glutamine, proline, creatine, putrescine, spermidine, spermine, arginine, ornithine, citrulline, fumarate, succinate, argininosuccinate, succinyl, ketoglutarate, aconitate, glyoxylate, caffeine, sugars, carbs, etc.). In some aspects, analyte sensor 1352 can measure one or more analytes simultaneously with one or more corresponding electrochemical biosensors for each different analyte measured.

On body electronics 1354 can be configured to process signals from analyte sensor 1352. On body electronics 1354 can be further configured to communicate data (e.g., sensor data) from analyte sensor 1352 to one or more external devices (e.g., remote device 1310, first injection pen 1360, second injection pen 1370, first smart cap 1382, second smart cap 1384, software application 1390, etc.). On body electronics 1354 can be further configured to wirelessly communicate (e.g., NFC, WiFi, Bluetooth, BLE, Internet, etc.) analyte related sensor data. As shown in FIG. 13, on body electronics 1354 can be operatively (e.g., electrically) coupled to analyte sensor 1352 and wirelessly coupled to remote device 1310, first injection pen 1360, second injection pen 1370, first smart cap 1382, second smart cap 1384, and/or software application 1390.

In some aspects, on body electronics 1354 can include a printed circuit board (PCB) for connection to various components (e.g., analyte sensor 1352, processor, ASIC, wireless transceiver, wireless transmitter, controller, memory, etc.). In some aspects, on body electronics 1354 can store (e.g., via memory) historical analyte related data. In some aspects, on body electronics 1354 can be configured to store some or all of analyte related data (e.g., sensor data) from analyte sensor 1352 in a memory. In some aspects, on body electronics 1354 can include one or more processors and/or control logic configured to determine (e.g., via software programs and/or algorithms) current analyte levels, rates of change (ROC) of analyte levels, rates of acceleration of analyte levels, and/or analyte trend information (e.g., trend display 1324), and/or analyte fluctuation levels (e.g., standard deviation, variability, variance, etc.).

In some aspects, on body electronics 1354 can be configured to send (broadcast) analyte related data (e.g., sensor data) to one or more external devices (e.g., remote device 1310, first injection pen 1360, second injection pen 1370, first smart cap 1382, second smart cap 1384, software application 1390, etc.). In some aspects, on body electronics 1354 can be configured to send (broadcast) real-time data associated with monitored analyte levels from analyte sensor 1352 to one or more external devices of therapy management system 1300, for example, when the external device is within a communication range (e.g., BLE range) of the data broadcast from OBU 1350.

In some aspects, on body electronics 1354 can be configured to wirelessly transmit stored analyte related sensor data during a monitoring time period (e.g., sensor wear) to one or more external devices of therapy management system 1300. In some aspects, analyte related data (e.g., sensor data) sent from on body electronics 1354 can be stored in one or more memory units (e.g., permanently, temporarily), for example, memory units on one or more external devices of therapy management system 1300. In some aspects, remote device 1310 can be configured as a data conduit to pass data received from on body electronics 1354 (e.g., sensor data) to one or more external devices. In some aspects, on body electronics 1354 can be designed to store the sensor data (e.g., glucose data) from analyte sensor 1352 collected over the course of the sensor wear period (e.g., 3 days, 7 days, 14 days, 30 days, etc.), for example, over 14 days.

On body housing 1355 can be configured to provide an interior compartment for a portion of analyte sensor 1352 (e.g., proximal portion) and on body electronics 1354. As shown in FIG. 13, on body housing 1355 can include analyte sensor 1352 and on body electronics 1354 and be coupled to adhesive layer 1356. In some aspects, on body housing 1355 can include a sealed housing (e.g., hermetically sealed biocompatible housing). Adhesive layer 1356 can be configured to attach OBU 1350 to a skin surface of a user. As shown in FIG. 13, adhesive layer 1356 can be coupled to on body housing 1355 to securely position a portion of analyte sensor 1352 (e.g., distal portion) to a skin surface.

First injection pen 1360 can be configured to administer one or more dosing regimen. In some aspects, first injection pen 1360 can be combined with first smart cap 1382 to transfer medication dose data to remote device 1310, OBU 1350, and/or software application 1390. As shown in FIG. 13, first injection pen 1360 can include first dial 1361 configured to set a dosage to be delivered and first dose indicator 1362 configured to indicate a dose. In some aspects, first injection pen 1360 can include a corresponding first smart cap 1382 in wireless communication with other components of therapy management system 1300. In some aspects, first injection pen 1360 can include an insulin injection pen for a certain type of insulin, such as a rapid-acting (RA) insulin pen. For example, first injection pen 1360 can include a Humalog™ pen, a Novolog™ pen, or an Apidra™ pen. In some aspects, first injection pen 1360 can include dose-capture technology, for example, to automatically capture injection data (e.g., set dose, injected dose, dose history, date, time of dose, time since last dose, type of medication, amount of medication remaining in pen, etc.), for example, via first smart cap 1382.

Second injection pen 1370 can be configured to administer one or more dosing regimen. In some aspects, second injection pen 1370 can be combined with second smart cap 1384 to transfer medication dose data to remote device 1310, OBU 1350, and/or software application 1390. As shown in FIG. 13, second injection pen 1370 can include second dial 1371 configured to set a dosage to be delivered and second dose indicator 1372 configured to indicate a dose. In some aspects, second injection pen 1370 can include a corresponding second smart cap 1384 in wireless communication with other components of therapy management system 1300. In some aspects, second injection pen 1370 can include an insulin injection pen for a certain type of insulin, such as a long-acting (LA) insulin pen. For example, second injection pen 1370 can include a Lantus™ pen, a Levemir™ pen, a Toujeo™ pen, or a Tresiba™ pen. In some aspects, second injection pen 1370 can include dose-capture technology, for example, to automatically capture injection data (e.g., dose, dose history, date, time, etc.), for example, via second smart cap 1384.

First smart cap 1382 can be configured to be in wireless communication with remote device 1310, OBU 1350, and/or software application 1390. First smart cap 1382 can be further configured to present information to a user, including, but not limited to, any information or sensor data as described elsewhere herein (e.g., an insulin dosage recommendation, notification, communication, etc.). In some aspects, first smart cap 1382 can exchange data with (e.g., retrieve sensor data from) OBU 1350, for example, by being brought in close proximity to OBU 1350. In some aspects, first smart cap 1382 can receive a meal size from the user (e.g., user announces the meal size, selects the meal size, etc.), including, but not limited to, a small meal, a medium meal, a large meal, or a snack. In some aspects, first smart cap 1382 can include at least one input control, for example, a button (e.g., electronic switch). The input control (e.g., button) can be configured to trigger first smart cap 1382 to perform one or more operations, including, but not limited to, any operation that is described elsewhere herein. As shown in FIG. 13, first smart cap 1382 can include first display 1383.

First display 1383 can include any suitable type of display technology, including, but not limited to, a dynamic electronic display (e.g., an LED display) or a static electronic display (e.g., an e-ink display). In some aspects, first display 1383 can include a user interface for obtaining a meal bolus recommendation for different types of meals (e.g., small, medium, large; breakfast, lunch, dinner, snack; 10 grams of carbs, 20 grams of carbs, 45 grams of carbs; etc.). In some aspects, first display 1383 can display first message 1331, second message 1332, third message 1333, first information display 1334, second information display 1335, or a combination thereof.

Second smart cap 1384 can be configured to be in wireless communication with remote device 1310, OBU 1350, and/or software application 1390. In some aspects, second smart cap 1384 can exchange data with (e.g., retrieve sensor data from) OBU 1350, for example, by being brought in close proximity to OBU 1350. In some aspects, second smart cap 1384 can receive a meal size from the user (e.g., user announces the meal size, selects the meal size, etc.), including, but not limited to, a small meal, a medium meal, a large meal, or a snack. In some aspects, second smart cap 1384 can include at least one input control, for example, a button (e.g., electronic switch). The input control (e.g., button) can be configured to trigger second smart cap 1384 to perform one or more operations, including, but not limited to, any operation that is described elsewhere herein. As shown in FIG. 13, second smart cap 1384 can include second display 1385.

Second display 1385 can include any suitable type of display technology, including, but not limited to, a dynamic electronic display (e.g., an LED display) or a static electronic display (e.g., an e-ink display). In some aspects, second display 1385 can include a user interface for obtaining a meal bolus recommendation for different types of meals (e.g., small, medium, large; breakfast, lunch, dinner, snack; 10 grams of carbs, 20 grams of carbs, 45 grams of carbs; etc.). In some aspects, second display 1385 can display first message 1331, second message 1332, third message 1333, first information display 1334, second information display 1335, or a combination thereof.

In some aspects, first smart cap 1382 and/or second smart cap 1384 can be configured to record, store, and/or convey one or more types of pen capping information, for example, information about when first smart cap 1382 and/or second smart cap 1384 is secured to and/or released from the corresponding first injection pen 1360 and/or second injection pen 1370, respectively. In some aspects, pen capping information can include information about a current capping period (e.g., the time since the last capping), information about a duration of one or more uncappings (which may also be referred to herein as “decapping(s)”), and/or the timing (e.g., time-of-day or time elapsed since) of each uncapping and each capping. For example, pen capping information can include data reflecting when first smart cap 1382 and/or second smart cap 1384 was placed onto the corresponding first injection pen 1360 and/or second injection pen 1370, when first smart cap 1382 and/or second smart cap 1384 was removed from the corresponding first injection pen 1360 and/or second injection pen 1370, respectively, or both. In some aspects, pen capping information can be presented to a user on a display of first smart cap 1382 and/or second smart cap 1384 (e.g., via first display 1383 and/or second display 1385). In some aspects, first smart cap 1382 and/or second smart cap 1384 can include a speaker, a microphone, a receiver, or other audio device, and pen capping information can be detected by the receiver (e.g., microphone) and/or presented by the speaker in first smart cap 1382 and/or second smart cap 1384.

In some aspects, for example, first smart cap 1382 and/or second smart cap 1384 can provide a timer clock that counts up from the last time first smart cap 1382 and/or second smart cap 1384 was secured to first injection pen 1360 and/or second injection pen 1370, respectively. In some aspects, first smart cap 1382 and/or second smart cap 1384 can wirelessly communicate pen capping information to one or more components of therapy management system 1300 (e.g., remote device 1310) and/or a remote computing device (e.g., remote server, remote computer, network, cloud server, smartphone, tablet, etc.). In some aspects, one or more accessories or smart delivery devices can detect other events associated with medication delivery actions of therapy management system 1300 and use that information in ways that pen capping information is described herein. For example, in some cases, an injection pen accessory (e.g., smart cap) may be secured to an injection pen such that it can detect the mechanical movement of the dosing mechanism to determine a time of a dose of medication and, optionally, but not necessarily, an amount of medication delivered at that time.

In some aspects, pen capping information can be stored, displayed, and/or analyzed in combination with glucose data to determine user behaviors, such as, for example, whether the user is appropriately dosing insulin for meals and/or to correct elevated glucose levels. In some aspects, pen capping information can be presented on a graphical representation of glucose data for a user and presented to the user and/or to a medical professional. In some aspects, glucose data from a period of time after each capping event of first smart cap 1382 and/or second smart cap 1384 can be evaluated to determine whether the user appropriately dosed insulin for that capping event, e.g., appropriate dose, underdose, overdose, overbasalization, etc.

In some aspects, a pen capping event can be disregarded where other information indicates that a dose was not provided. For example, where no change in the dosage selection of first injection pen 1360 and/or second injection pen 1370 (e.g., first dial 1361 and/or second dial 1371, respectively) was detected, the event may be disregarded. In some aspects, a pen uncapping and recapping event may be disregarded if the total uncapping time is less than a first threshold (e.g., about 4-6 seconds). For example, the first threshold may be determined based on an amount of time too short to permit for an injection, but long enough to allow a user to check the end of the pen to see if there is insulin remaining or if there is a needle attached to the pen. In some aspects, the total decapping time (e.g., the time between an uncapping event and the subsequent recapping) for a decapping event can be analyzed in combination with glucose data to determine if there was an injection during that decapping event. In some aspects, if the total decapping time exceeds a second threshold (e.g., at least 15 minutes, at least 30 minutes, etc.), glucose data and the methods described herein for detecting a meal timing can be used to determine an approximate time of an injection.

In some aspects, some or all components of therapy management system 1300 can perform one or more operations or functions as described elsewhere herein. In some aspects, therapy management system 1300 can evaluate insights based on reinforcement learning and perform one or more actions (e.g., one or more dosing regimen) for one or more triggered insights. In some aspects, therapy management system 1300 can determine insulin use by detecting removal and/or replacement of first smart cap 1382 and/or second smart cap 1384. In some aspects, therapy management system 1300 can detect an amount of insulin and/or other medication administered (e.g., GLP-1, prandial insulin, MDI of prandial insulin, etc.).

Software application 1390 can be configured to monitor one or more user metrics and implement one or more therapy pathways for a user (e.g., one or more dosing regimen). Software application 1390 can be further configured to provide therapy escalation pathways to manage diabetes and maintain one or more glucose metrics at a target level or in a target range. Software application 1390 can be further configured to guide a user with diabetes in managing their insulin therapy. Software application 1390 can be further configured to provide frequent therapy intervention and adjustment of therapy (e.g., on a daily or weekly basis rather than every 3-6 months). Software application 1390 can be further configured to continuously and/or periodically monitor one or more user metrics (e.g., mean glucose level, one or more glucose metrics, body weight, BMI, nausea, etc.) such that interventions to therapy can be timely and small. Software application 1390 can be further configured to continuously monitor and improve user adherence via coaching and direct feedback (e.g., notifications, warnings, recommendations, requests for user action, etc.), such that lack of adherence and lack of therapeutic effect of the treatment can be addressed separately. As shown in FIG. 13, remote device 1310 can include processor 1392 coupled to memory 1394 and configured to execute software application 1390.

As shown in FIG. 13, software application 1390 can be executed by one or more processors (e.g., processor, controller, microprocessor, microcontroller, ASIC, etc.), for example, processor 1392 on remote device 1310. In some aspects, software application 1390 can be coupled to a memory storing instructions, for example, memory 1394 on remote device 1310, instructions that when executed cause one or more processors to perform operations, including but not limited to, recommending a first dosing regimen of basal insulin, determining overbasalization of the first dosing regimen, recommending adding a second dosing regimen of GLP-1 or dual GIP/GLP-1, recommending adding a third dosing regimen of prandial insulin with one meal, recommending adding a fourth dosing regimen of stepwise MDI of prandial insulin, and recommending adding a fifth dosing regimen of basal insulin and prandial insulin with each meal.

In some aspects, software application 1390 can be part of remote device 1310. In some aspects, software application 1390 can be part of OBU 1350, first smart injection pen 1360, second smart injection pen 1370, first smart cap 1382, and/or second smart cap 1384. In some aspects, software application 1390 can include a mobile application (app). In some aspects, software application 1390 can include a web application (app). For example, software application 1390 can be part of a remote device (e.g., in a web app), for example, at a remote location (e.g., medical professional computer, service center, etc.).

In some aspects, processing and functionality required for software application 1390 can all be contained in a mobile app. In some aspects, some or part of software application 1390 (e.g., mobile app) can be contained in a remote server that supports software application 1390, for example, the remote server can support the mobile app with processing, communication hub, and/or reporting functionality. In some aspects, functionality described herein for software application 1390 includes functionality on the mobile app, remote server, or both, noting that all or some functionality can be in either or both. In some aspects, reference to software application 1390 described herein implies both a mobile app and a web server (e.g., remote server) supporting the mobile app.

In some aspects, software application 1390 (e.g., mobile app) can retrieve continuous glucose sensor data in real-time. In some aspects, software application 1390 can retrieve various combinations of discrete and continuous analyte measurements.

In some aspects, software application 1390 can be configured to evaluate one or more insights based on reinforcement learning and determine, for each of the insights, whether to generate that insight. In some aspects, each insight can be associated with performing one or more actions. For example, performing an action can include providing a message to the user and/or automatically changing the insulin therapy. In some aspects, the insights can be tailored to cover the user's entire therapy with diabetes, including, but not limited to, management of diabetes, maintaining one or more glucose metrics at a target level or in a target range, weight loss, one or more dosing regimen, and/or one or more therapy escalation pathways. In some aspects, the insights can include detection of observations from data (e.g., sensor data, user metrics, etc.) such that certain therapy actions can be taken. In some aspects, the insights are relevant enough to the overall therapy such that a user's therapy (e.g., MDI therapy) can be significantly improved.

In some aspects, software application 1390 can be configured to implement a therapy assessment of a current dosing regimen to monitor one or more user compliance metrics to confirm user adherence to the therapy, for example, for a first period of time (e.g., 3-day cycle). In some aspects, software application 1390 can be configured to implement a therapy assessment to rapidly titrate the basal insulin, for example, for a second period of time (e.g., 3-day cycle). In some aspects, rapidly titrating can include recommending an adjusted dose at a first interval (e.g., every day) based on the one or more user metrics for that first interval until no changes are made over a predetermined period of time (e.g., 3 days). In some aspects, software application 1390 can be configured to implement a therapy assessment to monitor no change in dosing output states, for example, for a third period of time (e.g., first 4 no change outputs in a row). In some aspects, software application 1390 can be configured to implement a therapy assessment to maintain titrating the basal insulin, for example, for a fourth period of time (e.g., 14-day cycle). In some aspects, maintaining titrating can include recommending an adjusted dose at a second interval (e.g., several days, a week, 2 weeks, etc.) based on the one or more user metrics for that second interval until a new issue is identified (e.g., new medication regimen, new exercise routine, new diet, etc.).

In some aspects, software application 1390 can be configured to determine overbasalization based on comparison of one or more metrics to predetermined thresholds. The thresholds may be pre-set by the system or may be adjustable by the user or medical professional. In some aspects, overbasalization may be determined based on a ratio of TDD to body weight. For example, the threshold may be based on a total insulin dose of greater than 0.5 units (U)/kg/day. The total daily dose (TDD) may be determined by summing the total amount of insulin taken by the user in one day. In some aspects, TDD may be manually entered by the user. In some aspects, a user may manually enter each insulin dose administered, and the system may calculate the TDD by adding up the individual doses. In some aspects, doses may be automatically recorded by a medication delivery device used to administer insulin, such as an insulin pump, a smart insulin pen, or a dose monitoring device installed on a medication delivery device, such as a smart pen cap, among others. Such devices may communicate the dose information to the system for calculation of the TDD. A combination of manually entered doses and automatically captured doses may be used to determine TDD. The body weight may be entered manually by a user into the computing device. In some aspects, a scale may communicate with the system. The user may weigh himself or herself using the scale, and the scale may automatically communicate the measured weight to the system. The system may periodically prompt the user to take a weight measurement, for example at the corresponding interval of the rapid titration phase or the maintenance titration phase.

In some aspects, software application 1390 can be configured to determine overbasalization based at least in part on a morning (AM)-bedtime (PM) glucose differential and/or postprandial-preprandial glucose differential, or a rate in change in overnight glucose levels or meal-time glucose levels, or a combination thereof.

In some aspects, software application 1390 can be configured to determine overbasalization based at least in part on a hypoglycemia metric exceeding a threshold The system may use glucose data to determine a hypoglycemia metric, such as time below range (TBR). The system may be configured to output glucose alarms to the user when the users glucose levels fall below a low glucose threshold, or below a very low glucose threshold. The system may track a number of low glucose alarms or very low alarms output during a predetermined period. Hypoglycemia may be determined if the number of alarms in the predetermined period exceeds a threshold.

In some aspects, software application 1390 can be configured to determine overbasalization based at least in part on an increase in glucose variability. Overbasalization may be detected if glucose variability exceeds a predetermined variability threshold. Glucose variability may be calculated using a coefficient of variation (e.g., ratio of standard deviation to the mean). In some aspects, the glucose variability threshold can include a coefficient of variation greater than 25%, 30%, or 35%, etc. In some aspects, glucose variability may be determined based on alternate measures of variability, such as standard deviation from the mean, or interquartile range, among others.

In some aspects, software application 1390 can be configured to implement one or more dosing regimen based on one or more targets of a user. In some aspects, for example, the target can be based at least in part on one or more metrics of the user. In some aspects, for example, the target can be based at least in part on a BMI of the user and a mean glucose level of the user. In some aspects, if one or more parameters of the user exceeds the one or more targets of the user, software application 1390 can be configured to escalate the therapy to maintain the one or more parameters at or within a range of the one or more targets by recommending adding and/or modifying one or more dosing regimen.

In some aspects, therapy management system 1300 can include a processor configured to execute software application (app) 1390 configured to monitor one or more user metrics and implement one or more therapy pathways for a user (e.g., one or more dosing regimen). In some aspects, software application 1390 can be part of remote device 1310, OBU 1350, first injection pen 1360, second injection pen 1370, first smart cap 1382, and/or second smart cap 1384. In some aspects, software application 1390 can include a mobile application on remote device 1310. In some aspects, therapy management system 1300 can include a network or a remote server configured to support software application 1390. In some aspects, software application 1390 can all be contained in a user mobile application (app) or some or part of software application 1390 can be contained in a remote server (e.g., web-server, cloud server, intranet server, etc.) that support the software application, for example, with processing, communication, and/or reporting functionalities. In some aspects, software application 1390 can include one or more application programming interfaces (APIs) for two or more computer programs to communicate with each other.

In some aspects, software application 1390 can be configured to recommend one or more therapy escalation pathways. In some aspects, software application 1390 can receive glucose data of a user from a CGM (e.g., OBU 1350), calculate one or more user metrics (e.g., as described herein), compare the one or more user metrics to one or more targets (thresholds) stored in memory that are pre-set or user-adjustable, and output a recommendation to the user for an adjusted dose and/or a new therapy to display 1312 of remote device 1310. In some aspects, software application 1390 can determine if the user actually takes the recommended dose and/or new therapy (e.g., monitoring one or more glucose metrics), determine one or more new metrics after the user takes the recommended dose, and recommend an adjusted (titrated) dose based on the glucose data. In some aspects, software application 1390 can recommend a titration phase in which software application 1390 requires a user to actively administer a recommended dose, reviews one or more user metrics after the user takes the medication, and then recommends a new dose based on glucose data of the user.

In some aspects, software application 1390 can be configured to determine a number of days for one or more dosing regimen (e.g., basal insulin regimen). For example, the number of days can be defined by a time between LA doses. In some aspects, software application 1390 can define a number of days for basal titration based on a time between LA doses. In some aspects, for example, a short day of less than a first interval (e.g., about 18 hours) between LA doses can be deemed invalid and discarded for determining days for basal titration. In some aspects, for example, a short day of more than a second interval (e.g., about 18 hours) between LA doses can be deemed valid and used for determining days for basal titration. In some aspects, for example, a long day of more than a third interval (e.g., about 24 hours) between LA doses can be deemed valid and used for determining days for basal titration. In some aspects, for example, a long day of more than a fourth interval (e.g., about 24 hours) between LA doses can be deemed a good day (e.g., 24 hour duration) and used for determining days for basal titration. In some aspects, for example, a long day of more than a fifth interval (e.g., about 42 hours) between LA doses can be deemed a missed dose day (e.g., 42 hours since last dose) and used for determining days for basal titration. In some aspects, a missed dose day can repeat for a sixth interval (e.g., every 24 hours) until another LA dose is taken and be used for determining days for basal titration.

### Exemplary Control Diagrams

FIG. 14A illustrates control diagram 1400A to implement one or more therapy pathways, according to various exemplary aspects. Control diagram 1400C can be configured to provide therapy escalation pathways to manage diabetes. Control diagram 1400C can be further configured to implement one or more therapy pathways for a user (e.g., one or more dosing regimen). Control diagram 1400C can be further configured to assess an adequacy of each dosing regimen and evaluate for overbasalization.

Although control diagram 1400A is shown in FIG. 14A as a stand-alone process, apparatus, and/or system, the aspects of this disclosure can be used with other processes, apparatuses, systems, and/or methods, such as, but not limited to, elements in FIGS. 13, 14B, 14C, 15-18, 19A-19C, and 20-23, e.g., therapy management system 1300, software application 1390, therapy assessment 1400B, control diagram 1400C, therapy assessment 1430, one or more dosing regimen 1410, 1440, 1450, 1460, 1480, flow diagram 2000, flow diagram 2100, flow diagram 2200, and/or computing device 2300.

As shown in FIG. 14A, control diagram 1400A can include a first therapy 1401, a therapy assessment 1403, and a second therapy 1405. In some aspects, control diagram 1400A can be implemented by software application 1390. In some aspects, control diagram 1400A can be implemented for one or more dosing regimen (e.g., one or more dosing regimen 1410, 1440, 1450, 1460, 1480) to provide one or more therapy escalation pathways. In some aspects, control diagram 1400A can include first dosing regimen 1410, second dosing regimen 1440, third dosing regimen 1450, fourth dosing regimen 1460, fifth dosing regimen 1480, or a combination thereof.

First therapy 1401 can be configured to recommend a first therapy if one or more user metrics is outside of one or more targets. In some aspects, first therapy 1401 can include first dosing regimen 1410 (e.g., basal insulin), second dosing regimen 1440 (e.g., GLP-1 or dual GIP/GLP-1), or a combination thereof. In some aspects, the one or more user metrics can include one or more glucose metrics (e.g., average glucose, median glucose, glucose TIR, glucose TBR, glucose TAR, glucose TVL, GMI, MMG, MPDG, PPG, BeAM, or a combination thereof), BBR, ISF, PRA-CG, body weight, BMI, TDD (e.g., of insulin), ratio of TDD to body weight, A1C level (e.g., percentage of glycated hemoglobin, mmol/ml), heart rate, blood pressure, or a combination thereof. In some aspects, the one or more targets can include a target glucose level (e.g., about 110 mg/dL), a target glucose level range (e.g., about 110 mg/dL to about 155 mg/dL), a target basal state (e.g., MMG of about 100 mg/dL to about 130 mg/dL), a target meal state (e.g., MPDG of about 100 mg/dL to about 120 mg/dL), a target correction ISF state (e.g., PRA-CG of about 70 mg/dL to about 180 mg/dL), a target body weight, a target A1C level (e.g., 5.7%), a target heart rate, a target blood pressure, or a combination thereof. In some aspects, the one or more targets can be based at least in part on a BMI and a mean glucose level of the user.

Therapy assessment 1403 can be configured to assess the adequacy of first therapy 1401 and evaluate for overbasalization. Therapy assessment 1403 can be further configured to confirm user adherence to first therapy 1401. In some aspects, therapy assessment 1403 can be configured to determine overbasalization based at least in part on medication delivery information. For example, overbasalization can be determined based at least in part on a ratio of TDD of insulin to a body weight of the user exceeding a predetermined threshold (e.g., exceeding about 0.5 U/kg/day). In some aspects, therapy assessment 1403 can be configured to determine overbasalization based at least in part on one or more glucose metrics. For example, overbasalization can be determined based at least in part on a change in glucose level in an overnight period (e.g., a morning (AM)-bedtime (PM) differential), a change in glucose level in a meal period (e.g., a postprandial-preprandial differential), a hypoglycemia metric subceeding a predetermined threshold (e.g., a glucose concentration of about 70 mg/dL), a glucose variability exceeding a predetermined threshold (e.g., pre-meal rises exceeding about 30 mg/dL, post-meal spikes exceeding about 110 mg/dL), or a combination thereof.

Second therapy 1403 can be configured to recommend adding a second therapy if the one or more user metrics remains outside of the one or more targets or if overbasalization is determined. In some aspects, second therapy 1403 can include second dosing regimen 1440 (e.g., GLP-1 or dual GIP/GLP-1), third dosing regimen 1450 (e.g., prandial insulin with one meal), fourth dosing regimen 1460 (e.g., stepwise MDI of prandial insulin), fifth dosing regimen 1480 (e.g., basal insulin and prandial insulin with each meal), or a combination thereof.

FIG. 14B illustrates therapy assessment 1400B, according to an exemplary aspect. Therapy assessment 1400B can be configured to assess adequacy of a current therapy, confirm user adherence, and recommend adjusting one or more dose amounts based on one or more user metrics. Therapy assessment 1400B can be further configured to be implemented for any of the dosing regimen described herein (e.g., one or more dosing regimen 1410, 1440, 1450, 1460, 1480).

Although therapy assessment 1400B is shown in FIG. 14B as a stand-alone process, apparatus, and/or system, the aspects of this disclosure can be used with other processes, apparatuses, systems, and/or methods, such as, but not limited to, elements in FIGS. 13, 14A, 14C, 15-18, 19A-19C, and 20-23, e.g., therapy management system 1300, software application 1390, control diagram 1400A, control diagram 1400C, therapy assessment 1430, one or more dosing regimen 1410, 1440, 1450, 1460, 1480, flow diagram 2000, flow diagram 2100, flow diagram 2200, and/or computing device 2300.

As shown in FIG. 14B, therapy assessment 1400B can include monitoring phase 1402, rapid titration phase 1404, and maintenance titration phase 1406. In some aspects, therapy assessment 1400B can be implemented by software application 1390. In some aspects, therapy assessment 1400B can be implemented for one or more dosing regimen (e.g., one or more dosing regimen 1410, 1440, 1450, 1460, 1480) to assess the current therapy and adjust the dose amount based on one or more user metrics.

Monitoring phase 1402 can be configured to monitor one or more user compliance metrics of a current therapy over a predetermined period of time. Monitoring phase 1402 can be further configured to determine whether the user is properly wearing and using a CGM (e.g., OBU 1350) and properly and consistently administering medication. In some aspects, the one or more user compliance metrics can include a frequency of glucose data (scans), time between gaps in glucose data, time of dose, amount of dose (e.g., relative to recommended dose), receipt of user input, or a combination thereof. In some aspects, the predetermined period of time can be in a range from about 1 day to about 7 days, for example, 3 days. In some aspects, once the one or more user compliance metrics are received (met) for the predetermined period of time (e.g., 3 days), the current therapy can proceed to rapid titration phase 1404.

Rapid titration phase 1404 can be configured to recommend an adjusted dose for a first interval (e.g., each day at midnight) based on one or more user metrics (e.g., listed above) during that first interval. In some aspects, rapid titration phase 1404 can continue the above process until no changes are made over a predetermined period of time (e.g., 3 days, 6 days over 7 days, etc.). In some aspects, dose adjustments can be at fixed intervals (e.g., 0.25 U increase, 10% increase, etc.) or can be proportional to the degree of glycemic dysfunction (e.g., higher dose changes if significantly outside of glucose targets). In some aspects, once no changes are received for the predetermined period of time (e.g., 3 days, 6 days over 7 days, etc.), the current therapy can proceed to maintenance titration phase 1406.

Maintenance titration phase 1406 can be configured to recommend an adjusted dose for a second interval (e.g., several days, a week) based on one or more user metrics (e.g., listed above) during that second interval. In some aspects, maintenance titration phase 1406 can continue the above process until a new issue with the current therapy is identified. In some aspects, dose adjustments can be at fixed intervals (e.g., 0.25 U increase, 10% increase, etc.) or can be proportional to the degree of glycemic dysfunction (e.g., higher dose changes if significantly outside of glucose targets). In some aspects, the new issue can include a new medication, new exercise routine, new diet, or a combination thereof.

### Exemplary Dosing Regimen

FIGS. 14C, 15-18 and 19A-19C illustrate control diagram 1400C to implement one or more dosing regimen 1410, 1440, 1450, 1460, 1480, according to various exemplary aspects. Control diagram 1400C can be configured to provide therapy escalation pathways to manage diabetes. Control diagram 1400C can be further configured to implement one or more therapy pathways for a user (e.g., one or more dosing regimen). Control diagram 1400C can be further configured to assess an adequacy of each dosing regimen and evaluate for overbasalization.

Although control diagram 1400C is shown in FIG. 14C as a stand-alone process, apparatus, and/or system, the aspects of this disclosure can be used with other processes, apparatuses, systems, and/or methods, such as, but not limited to, elements in FIGS. 13, 14A, 14B, 15-18, 19A-19C, and 20-23, e.g., therapy management system 1300, software application 1390, control diagram 1400A, therapy assessment 1400B, therapy assessment 1430, one or more dosing regimen 1410, 1440, 1450, 1460, 1480, flow diagram 2000, flow diagram 2100, flow diagram 2200, and/or computing device 2300.

As shown in FIG. 14C, control diagram 1400C can include first dosing regimen 1410 (e.g., basal insulin), therapy assessment 1430, second dosing regimen 1440 (e.g., GLP-1 or dual GIP/GLP-1), third dosing regimen 1450 (e.g., prandial insulin with one meal), fourth dosing regimen 1460 (e.g., stepwise MDI of prandial insulin), and/or fifth dosing regimen 1480 (e.g., basal insulin and prandial insulin with each meal). In some aspects, control diagram 1400C can implement one or more therapies to a user that include one or more of first dosing regimen 1410, therapy assessment 1430, second dosing regimen 1440, third dosing regimen 1450, fourth dosing regimen 1460, fifth dosing regimen 1480, or a combination thereof. In some aspects, control diagram 1400C can be implemented by software application 1390.

In some aspects, for example, software application 1390 can monitor user data (e.g., one or more user metrics) of therapy management system 1300 (e.g., via OBU 1350, first injection pen 1360, second injection pen 1370, first smart cap 1382, and/or second smart cap 1384), send one or more control signals or instructions to therapy management system 1300 (e.g., via remote device 1310, first smart cap 1382, and/or second smart cap 1384) to perform or instruct the user to perform one or more dosing regimen of control diagram 1400C, and assess whether any overbasalization has occurred for the one or more dosing regimen.

First dosing regimen 1410 can be configured to initiate basal insulin therapy. In some aspects, if one or more user metrics is outside of one or more targets (e.g., average glucose, median glucose, glucose TIR, glucose TBR, glucose TVL, GMI, MMG, PPG, BBR, BeAM, ISF, weight, BMI, A1C level, heart rate, blood pressure, or a combination thereof), software application 1390 can implement first dosing regimen 1410. In some aspects, the one or more targets can include a target basal state (e.g., MMG of about 100 mg/dL to about 130 mg/dL). In some aspects, first dosing regimen 1410 can be based at least in part on user-specific considerations, for example, choice of basal insulin can be based on cost and any pre-existing conditions of the user. In some aspects, first dosing regimen 1410 can be based at least in part on a prescription of glucagon for emergent hypoglycemia. In some aspects, first dosing regimen 1410 can have an initial starting dose of basal insulin of about 0.1 U/kg/day to about 0.2 U/kg/day. In some aspects, first dosing regimen 1410 can include adding a CGM (e.g., OBU 1350) to the user to set an initial glucose target (e.g., glucose target can be in a range of about 117 mg/dL to about 154 mg/dL).

In some aspects, first dosing regimen 1410 can include a monitoring phase to ensure the user is properly using the CGM and is complying with the therapy regimen. This may be determined by monitoring one or more of system use, sensor wear, sensor scanning or completeness of glucose data, and dosing administered by the user (correspondence of dose administered to recommended dose). For example, the monitoring phase can be for a first time period. The first time period may be one or more days. One or more compliance metrics may be assessed on a daily basis, wherein therapy adherence is determined for each day based on the compliance metrics. One a compliance threshold is reached, such as 3 days in a row where compliance targets are met, the user may be deemed to adhere to therapy. In some aspects, if the user is not adhering to first dosing regimen 1410, software application 1390 can provide coaching and interventions to help the user adhere to first dosing regimen 1410 more effectively. For example, the coaching can include alarms and reminders (e.g., message to remind the user to take the therapy), education or encouragement (e.g., message to advise the user of the importance of regular therapy use, encouragement to continue to use the therapy), and/or long form education (e.g., general in-person or virtual diabetes education to better understand what is happening to the user's body and the value of using the therapy). The notifications may be based on the compliance metrics. For example, if the compliance metric relates to timing of insulin doses, and the user is not taking the insulin dose at a consistent time, the notification may alert the user, and provide a recommendation to take the dose at a particular time. If the compliance metric indicates incomplete glucose data (e.g., glucose data for 70% or less of the day), the notification may be to alert the user to ensure the sensor is worn and the remote device is kept in range, or to scan the sensor to obtain glucose data more frequently.

In some aspects, the coaching can be user directed. For example, the coaching can include short educational messages (e.g., “Did you know that BEHAVIOR A is associated with OUTCOME Y,” “Why don't you take a 45 minute walk today,” “Did you know a walk can MESSAGE Z”), enrollment in adherence alarms or alerts (e.g., missed dose alerts, low alerts, get moving alerts), motivational messages to encourage behavior (e.g., “You scanned your CGM at least 4 times EVERY day for the last week, keep it up!”, “You took your DRUG A on time 95% of the time over the last month, on days where you took DRUG A, you told us you felt better than on days where you did not”), and/or long form education (e.g., take a cooking class, enroll in a virtual Type-2 diabetes education class).

In some aspects, the intervention can be medical professional directed. For example, the intervention can include user is not taking medication every day so encourage user to take medication (e.g., no need to escalate therapy), user is not taking medication so consider an easier medication to dose (e.g., once-a-day medication, oral vs. injectable, etc.), user is taking medication but not seeing therapeutic benefits so consider intensifying current therapy (e.g., patterns of physiological markers do not match expected behavior, insulin not dropping glucose levels, GLP-1 not reducing weight), user is taking medication but not seeing therapeutic benefits so consider adding therapy, and/or user is taking medication but not seeing therapeutic benefits so consider an alternative therapy (e.g., basal insulin→basal insulin+GLP-1).

In some aspects, software application 1390 can define a preferred therapy algorithm (e.g., control diagram 1400A, control diagram 1400C). For example, the preferred therapy algorithm can be defined by the user (e.g., user's value and goals) or by one or more medical professionals. In some aspects, for example, the preferred therapy algorithm can include a predetermined therapy escalation pathway (e.g., use diet and exercise→metformin→metformin+GLP-1→GLP-1+basal insulin→GLP-1+MDI). In some aspects, for example, the preferred therapy algorithm can remove specific medications and/or omit certain therapy pathways from consideration (e.g., do not use SGLT-2 agents or flozins, do not use diet and exercise, etc.). In some aspects, software application 1390 can allow a medical professional to specify what changes to the preferred therapy algorithm can be made directly by the user (e.g., user can increase insulin dose by 20%, user can increase GLP-1 dose by 1-step (10 pg for 2 weeks), etc.).

In some aspects, if the user is adhering to first dosing regimen 1410 but not seeing a therapeutic effect, software application 1390 can provide one or more options to adjust the therapy (e.g., therapy pathways). For example, for a user on injectable medications, software application 1390 can adjust the dosing (e.g., basal insulin dose). For example, for a user on oral medications, software application 1390 can send messages to the user's HCP to adjust the dosing of the oral medications and/or request adding other oral medications to the user's current therapy. For example, software application 1390 can escalate the user's therapy to higher intensity medication (e.g., basal insulin→MDI, metformin→GLP-1, etc.). In some aspects, if the user is adhering to first dosing regimen 1410 but not seeing a therapeutic effect, software application 1390 can implement second dosing regimen 1440 (e.g., basal insulin→basal insulin+GLP-1).

Therapy assessment 1430 can be configured to assess the adequacy of one or more dosing regimen and evaluate for overbasalization. Therapy assessment 1430 can be further configured to determine overbasalization by comparing one or more metrics to one or more thresholds for those metrics. In some aspects, therapy assessment 1430 can be performed for first dosing regimen 1410. In some aspects, therapy assessment 1430 can determine overbasalization based at least in part on a ratio of total daily dose (TDD) of insulin to a body weight of the user. In some aspects, for example, the ratio of TDD of insulin to the body weight can be compared to a predetermined threshold (e.g., about 0.5 U/kg/day). In some aspects, therapy assessment 1430 can determine overbasalization based at least in part on glucose variability. In some aspects, for example, glucose variability can be compared to one or more predetermined thresholds (e.g., pre-meal rise of about 30 mg/dL, post-meal spike of about 110 mg/dL, total change in glucose level from a pre-meal period to a post-meal period). In some aspects, therapy assessment 1430 can determine overbasalization based at least in part on a hypoglycemia metric. In some aspects, for example, the hypoglycemia metric can include a low blood glucose index (LBGI), time below range (TBR), number of low glucose alarms, number of very low glucose alarms, among other hypoglycemia indicators. In some aspects, for example, the hypoglycemia metric can be compared to a predetermined threshold (e.g., glucose concentration below about 70 mg/dL). In some aspects, therapy assessment 1430 can determine overbasalization based at least in part on a change in glucose level in a meal period. In some aspects, for example, the change in glucose level in the meal period can be compared to a predetermined threshold (e.g., postprandial-preprandial differential). In some aspects, therapy assessment 1430 can determine overbasalization based at least in part on a change in glucose level in an overnight period. In some aspects, for example, the change in glucose level in the overnight period can be compared to a predetermined threshold (e.g., morning (AM)-bedtime (PM) differential).

If overbasalization is detected, software application 1390 may be configured to recommend that the user initiate a second dosing regimen. Remote device 1310 may output a notification on display 1312 to provide a recommendation to start a second dosing regimen. The system may alternatively or additionally provide a notification to a computing device of the HCP to recommend initiating the second dosing regimen. In some aspects, computing device may generate a report that includes the recommended dose regimen.

Second dosing regimen 1440 may include addition of a GLP-1 to the existing therapy, e.g., basal insulin therapy. GLP-1 may be added if GLP-1 is not counter-indicated for the patient, and is not already part of the user's therapy regimen. System may retrieve current therapy information to determine if the therapy regimen already includes a GLP-1. System may include information regarding counter-indications, which may be entered by the user or HCP to determine if GLP-1 is counter-indicated.

In some aspects, upon initiation of GLP-1 receptor agonist (RA) therapy, basal insulin is subject to an initial reduction of a predetermined amount. For example, the current basal insulin dose may be reduced by 20% of the current basal insulin dose. The basal insulin dose may be adjusted, by further decreasing or increasing the basal insulin dose during the rapid titration phase as described herein.

GLP-1 RA may be titrated simultaneously or sequentially with basal insulin or other existing therapies. GLP-1 RA may be titrated between approved ranges of the particular GLP-1 RA. GLP-1 RA may be initiated at a first dose and may be increased incrementally toward a maximum dose. The dose may be increased incrementally, e.g., on a unit, or fractional unit basis as long as GLP-1 RA is tolerated by the user.

In some aspects, the system may recommend increasing the dose of GLP 1 RA when no side effects, such as nausea, are reported or detected. The system may prompt the user for input on side-effects. The system may specifically ask if the user is experiencing nausea or vomiting. The system may ask how the user is feeling by free-form text. The system may ask the user to select from a predetermined list of side-effects. In some aspects, the system may be configured to detect nausea automatically (e.g., via an external sensor). If nausea is reported, the dose of GLP-1 RA may be maintained at the current level and no further increase is recommended. Alternatively, if nausea is reported, the dose of GLP-1 RA may return to the previous dose of GLP-1 RA administered that did not result in nausea as reported by the user.

In some aspects, GLP-1 RA is also titrated based on the user's body weight. The system may determine a target weight for the user. The target weight may be input by the user, such as based on input from an HCP, or may be input directly by the HCP, or may be a default setting. The target weight may be based on BMI. The user's weight may be determined by a smart scale. The smart scale may automatically communicate weight measurements to the system. The system may prompt the user to weigh himself or herself. The user may be prompted to take a weight measurement. The user may be prompted on a periodic basis, once every two days, once every three days, once per week, among other intervals. Alternatively, the system may prompt the user for a weight measurement if no weight measurement has been received for a predetermined period of time. If the user's weight drops below the target weight, no further increases to GLP-1 RA may be recommended in order to avoid excessive weight loss. Alternatively, if the user's weight drops below the target weight, the GLP-1 RA dose may be decreased to inhibit further weight loss.

Second dosing regimen 1440 can be configured to add GLP-1 receptor agonists therapy or dual GIP/GLP-1 receptor agonists therapy. In some aspects, if one or more user metrics is above one or more targets (e.g., average glucose, median glucose, glucose TIR, glucose TBR, glucose TVL, GMI, MMG, PPG, BBR, BeAM, ISF, weight, BMI, A1C level, heart rate, blood pressure, or a combination thereof), software application 1390 can implement second dosing regimen 1440. In some aspects, the one or more targets can include a target basal state (e.g., MMG of about 100 mg/dL to about 130 mg/dL), a target weight, a target meal state (e.g., minimum post-dose glucose (MPDG) of about 100 mg/dL to about 120 mg/dL), a target correction ISF state (e.g., post rapid-acting dose with correction glucose (PRA-CG) of about 70 mg/dL to about 180 mg/dL), or a combination thereof. In some aspects, second dosing regimen 1440 can add GLP-1 receptor agonists or dual GIP/GLP-1 receptor agonists in free combination or in a fixed-ratio combination with insulin (e.g., basal insulin). In some aspects, second dosing regimen 1440 (e.g., GLP-1 or dual GIP/GLP-1) can be added to first dosing regimen 1410 (e.g., basal insulin). For example, if one or more user metrics remains above one or more targets after first dosing regimen 1410 or if overbasalization is determined for first dosing regimen 1410, software application 1390 can recommend adding second dosing regimen 1440 to first dosing regimen 1410. In some aspects, second dosing regimen 1440 (e.g., GLP-1 or dual GIP/GLP-1) can be recommended to be added to first dosing regimen 1410 (e.g., basal insulin). For example, if one or more user metrics remains above one or more targets after first dosing regimen 1410 or if overbasalization is determined for first dosing regimen 1410, software application 1390 can recommend adding second dosing regimen 1440 to first dosing regimen 1410.

If overbasalization is detected, and the user's therapy regimen already includes GLP-1 RA and is fully titrated, or GLP-1 RA is counter-indicated, the system determines whether the user has a high pre-to-post-meal glucose differential for one meal. The pre-to-post-meal glucose differential may be based on the change in glucose level from the glucose level at the start of a meal or a time before the start of a meal and the maximum glucose level following the meal, or the maximum glucose level in a predetermined time period following the meal (e.g., 2-5 hours).

Third dosing regimen 1450 can be configured to add prandial insulin therapy with one meal. In some aspects, if a post-meal glucose rise for one meal exceeds a predetermined threshold, software application 1390 can implement third dosing regimen 1450. In some aspects, the predetermined threshold can be defined by a total change (delta between minimum and maximum) in glucose level from a pre-meal period to a post-meal period, a rate of change (slope) in glucose level, a postprandial-preprandial differential, or a combination thereof. In some aspects, prandial insulin therapy can include bolus insulin, RA insulin, “mealtime” insulin, aspart, glulisine, lispro, or a combination thereof. In some aspects, third dosing regimen 1450 (e.g., prandial insulin with one meal) can include one dose of prandial insulin with the user's largest meal. In some aspects, third dosing regimen 1450 (e.g., prandial insulin with one meal) can include one dose of prandial insulin with the user's meal having the greatest PPG excursion. In some aspects, third dosing regimen 1450 (e.g., prandial insulin with one meal) can be recommended to be added to second dosing regimen 1440 (e.g., GLP-1 or dual GIP/GLP-1). For example, if a post-meal glucose rise for one meal exceeds a predetermined threshold after second dosing regimen 1440 or if overbasalization is determined for second dosing regimen 1440, software application 1390 can recommend adding third dosing regimen 1450 to second dosing regimen 1440.

In some aspects, third dosing regimen 1450 can have an initial starting dose of prandial insulin with one meal of a predetermined amount, or based on a ratio or proportion of prandial insulin to basal insulin. For example, an initial prandial insulin dose may be about 4 U/day or about 10% of once-daily basal insulin dose. In some aspects, if the user's A1C level is less than 8% (e.g., about 64 mmol/mol), third dosing regimen 1450 can recommend lowering the basal insulin dose by about 4 U/day or by about 10% of once-daily basal insulin dose. In some aspects, for hypoglycemia, software application 1390 can determine the cause of hypoglycemia by analyzing one or more user metrics, such as based on a low blood glucose index (LBGI), time below range (TBR), number of low glucose alarms, number of very low glucose alarms, among other hypoglycemia indicators. In some aspects, if there is no clear reason for occurrence of hypoglycemia (e.g., based on above metrics), for safety reasons, the dose of once-daily insulins can be lowered by about 10% to about 20%.

In some aspects, the prandial dose is added to the meal that is providing the glucose excursion, such as the meal having the highest pre-to-post meal glucose rise. In some aspects, the prandial dose is added to the largest meal of the day.

Fourth dosing regimen 1460 can be configured to add stepwise MDI of prandial insulin therapy with corresponding meals. If the system determines based on glucose data that there is a high pre-to-post-meal glucose differential for more than one meal, the system may recommend adding multiple daily injections (MDI). Rather than adding a prandial insulin dose for a single meal, prandial insulin doses may be added for two meals, or for each of the three main meals in a day (i.e., breakfast, lunch and dinner). In some aspects, MDI is the final therapy escalation recommended by the system.

In some aspects, the one or more targets can include a target basal state (e.g., MMG of about 100 mg/dL to about 130 mg/dL and BBR<1.5), a target meal state (e.g., MPDG of about 100 mg/dL to about 120 mg/dL), a target correction ISF state (e.g., PRA-CG of about 70 mg/dL to about 180 mg/dL), or a combination thereof. In some aspects, fourth dosing regimen 1460 can include stepwise MDI of prandial insulin such that the therapy includes two and then three additional injections daily (e.g., with each meal). In some aspects, fourth dosing regimen 1460 (e.g., stepwise MDI of prandial insulin) can be recommended to be added to third dosing regimen 1450 (e.g., prandial insulin with one meal). For example, if one or more user metrics remains above one or more targets after third dosing regimen 1450, software application 1390 can recommend adding fourth dosing regimen 1460 to third dosing regimen 1450.

Fifth dosing regimen 1480 can be configured to add basal insulin and prandial insulin therapy with each meal. In some aspects, if one or more user metrics is above one or more targets (e.g., average glucose, median glucose, glucose TIR, glucose TBR, glucose TVL, GMI, MMG, PPG, BBR, BeAM, ISF, weight, BMI, A1C level, heart rate, blood pressure, or a combination thereof), software application 1390 can recommend adding fifth dosing regimen 1480. In some aspects, the one or more targets can include a target basal state (e.g., MMG of about 100 mg/dL to about 130 mg/dL and BBR<1.5), a target meal state (e.g., MPDG of about 100 mg/dL to about 120 mg/dL), a target correction ISF state (e.g., PRA-CG of about 70 mg/dL to about 180 mg/dL), or a combination thereof. In some aspects, fifth dosing regimen 1480 (e.g., basal insulin and prandial insulin therapy with each meal) can be recommended to be added to fourth dosing regimen 1460 (e.g., stepwise MDI of prandial insulin). For example, if one or more user metrics remains above one or more targets after fourth dosing regimen 1460, software application 1390 can recommend adding fifth dosing regimen 1480 to fourth dosing regimen 1460.

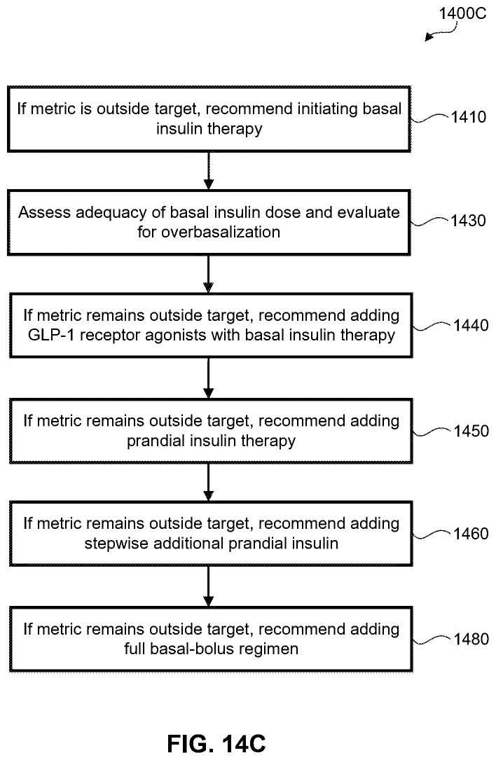

FIG. 15 illustrates therapy assessment 1430, according to an exemplary aspect. Although therapy assessment 1430 is shown in FIG. 15 as a stand-alone process, apparatus, and/or system, the aspects of this disclosure can be used with other processes, apparatuses, systems, and/or methods, such as, but not limited to, elements in FIGS. 13, 14A-14C, 16-18, 19A-19C, and 20-23, e.g., therapy management system 1300, software application 1390, control diagram 1400A, therapy assessment 1400B, control diagram 1400C, one or more dosing regimen 1410, 1440, 1450, 1460, 1480, flow diagram 2000, flow diagram 2100, flow diagram 2200, and/or computing device 2300.

As shown in FIG. 15, therapy assessment 1430 can include monitoring phase 1432, rapid titration phase 1434, no change phase 1436, and maintenance titration phase 1438. In some aspects, therapy assessment 1430 can be based at least in part on a ratio of TDD of insulin to a body weight of the user, glucose variability, a hypoglycemia metric, a change in glucose level in a meal period or an overnight period, or a combination thereof. In some aspects, therapy assessment 1430 can be based at least in part on a ratio of TDD of insulin to a body weight of the user exceeding a predetermined threshold (e.g., a basal insulin dose exceeding about 0.5 U/kg/day), an increase in morning (AM)-bedtime (PM) differential and/or postprandial-preprandial differential, a hypoglycemia metric subceeding a predetermined threshold, a glucose variability exceeding a predetermined threshold (e.g., pre-meal rises exceeding about 30 mg/dL and/or post-meal spikes exceeding about 110 mg/dL), or a combination thereof.

Monitoring phase 1432 can be configured to monitor one or more user compliance metrics of a current therapy over a predetermined period of time to ensure user adherence of the therapy. In some aspects, software application 1390 can monitor one or more of system use, sensor wear, scanning of analyte sensor, administration of medication doses, or a combination thereof by wirelessly tracking sensor data over time (e.g., via OBU 1350) to ensure the user is wearing the sensor enough and scanning the sensor enough (e.g., tracking number of scans with remote device 1310, glucose data is collected for a predetermined proportion of the day, glucose TBR, glucose TVL, number of low events, etc.), and by wirelessly tracking medication (dosing) data over time (e.g., via a medication delivery device, first smart cap 1382) to ensure the user is properly dosing (e.g., tracking doses per week, time between doses, dose regularity, etc.).

In some aspects, software application 1390 can implement monitoring phase 1432 until a compliance threshold is met. The compliance threshold may be based on one or more compliance metrics (e.g., as described herein) being met for a predetermined period of time, for example, 3 consecutive days. In some aspects, monitoring phase 1432 can continue to be implemented, with no therapy changes or escalations being permitted, until user adherence to the current therapy (e.g., compliance threshold is met) is observed for a period of time, for example, for 3 days in a row.

Once monitoring phase is complete, application proceeds to a rapid titration phase 1434. During the rapid titration phase 1434, the system recommends a medication dose at a first interval, such as once per day. Rapid titration phase 1434 can titrate the dose by fixed increments, e.g., increasing or decreasing by a fixed number of units, or by a fixed percentage of the current dose. In some aspects, rapid titration phase 1434 may be configured to apply a risk based rapid dose adjustment of first dosing regimen 1410. In some aspects, the dose adjustment may be proportional to the deviation of glucose metrics from the corresponding targets, for example, if the glucose metric is far from the target, a largest dose adjustment may be recommended than if the glucose metric is closer to the target. In some aspects, the user administers the medication dose, and system can determine one or more user metrics in the period following administration of the dose to determine if the user's glucose control is improved and is within a target level or within a target range for one or more glucose metrics. If glucose metrics are not within the target range, the system may recommend increasing or decreasing the medication dose. The system may also monitor one or more medication (dosing) data during rapid titration phase 1434 to titrate the medication within the standard dosing range of the medication. The system may generate a notification to the user and/or to the HCP to adjust the dose of medication.

In some aspects, during rapid titration phase 1434, software application 1390 can recommend a dose adjustment at a first interval, such as on a daily basis or 24 hour basis, to basal insulin. In some aspects, software application 1390 can monitor the user's reaction to the therapy during rapid titration phase 1434 by determining one or more glucose metrics as discussed herein, and comparing the metrics to target levels or ranges. For example, the system may wirelessly track sensor glucose data over time (e.g., tracking how far from target range fasting or morning glucose is, glucose TAR, glucose TBR, etc.) and medication (dosing) data over time (e.g., tracking dosed LA insulin the day before, no overnight treatments of hyperglycemia and/or hypoglycemia, sufficient overnight and/or morning data, sufficient time between last RA dose and morning glucose, etc.). In some aspects, during rapid titration phase 1434, any day with excessive low glucose, as may be determined based on TBR exceeding a TBR threshold, a number of low glucose alarms exceeding a predetermined number of low glucose alarms, will result in software application 1390 recommending decreasing a dose of basal insulin by a predetermined amount (e.g., decreased by about 10% to about 20%) in attempt to remedy the low glucose.

In some aspects, software application 1390 can implement rapid titration phase 1434 for a period of time, for example, for a 3-day cycle. In some aspects, rapid titration phase 1434 can be implemented for 3 days of dosing (e.g., basal insulin) in a row with sufficient glucose. In some aspects, rapid titration phase 1434 can continue to be implemented, with no therapy changes or escalations being permitted, until the user exhibits sufficient glucose control (e.g., glucose metrics at or within a range of one or more targets) for a predetermined period of time, for example, for 3 days of dosing in a row with sufficient glucose.

No change phase 1436 can be configured to monitor the user's reaction to first dosing regimen 1410 (e.g., basal insulin). No change phase 1436 can be further configured to monitor any side-effects of first dosing regimen 1410. In some aspects, software application 1390 can monitor one or more basal states 1436a-1436h and a range from a target basal state (e.g., MMG of about 100 mg/dL to about 130 mg/dL) during no change phase 1436. In some aspects, software application 1390 can monitor the user's reaction to the therapy during no change phase 1436 by wirelessly tracking sensor data over time (e.g., tracking overall glucose TBR, glucose TVL, MMG, BeAM, overbasalization indicators, etc.) and by wirelessly tracking medication (dosing) data over time (e.g., tracking treated overnight low, etc.).

In some aspects, software application 1390 can implement no change phase 1436 for a period of time, for example, for a no change cycle. In some aspects, no change phase 1436 can be implemented for 4 consecutive no change outputs (e.g., basal states 1436a-1436h) in a row. In some aspects, no change phase 1436 can continue to be implemented, with no therapy changes or escalations being permitted, until the user exhibits no change (e.g., no side-effects) for a sequential period of time, for example, for the first 4 no change outputs in a row.

As shown in FIG. 15, no change phase 1436 can be based on consecutive no changes to one or more basal states 1436a-1436h. In some aspects, one or more basal states 1436a-1436h can include first basal state 1436a (e.g., excessively below target), second basal state 1436b (e.g., significantly below target), third basal state 1436c (e.g., below target), fourth basal state 1436d (e.g., target), fifth basal state 1436e (e.g., above target), sixth basal state 1436f (e.g., significantly above target), seventh basal state 1436g (e.g., excessively above target, saturation), eighth basal state 1436h (e.g., overbasalization), or a combination thereof.

In some aspects, first basal state 1436a can be determined based on an overall glucose TBR of greater than about 4% or glucose TVL of less than about 1%. In some aspects, second basal state 1436b can be determined based on a MMG of less than about 80 mg/dL or a treated overnight low greater than about 30 minutes. In some aspects, third basal state 1436c can be determined based on a MMG of about 80 mg/dL to about 100 mg/dL or a BeAM of less than about −20 mg/dL/hr. In some aspects, fourth basal state 1436d can be determined based on a MMG of about 100 mg/dL to about 130 mg/dL. In some aspects, fifth basal state 1436e can be determined based on a MMG of about 130 mg/dL to about 150 mg/dL. In some aspects, sixth basal state 1436f can be determined based on a MMG of about 150 mg/dL to about 180 mg/dL. In some aspects, seventh basal state 1436g can be determined based on a MMG of greater than about 180 mg/dL. In some aspects, eighth basal state 1436h can be determined by therapy assessment 1430 or by one or more overbasalization metrics described above (e.g., ratio of TDD to body weight, glucose variability, hypoglycemia metric, change in glucose in meal period, change in glucose in overnight period).

Once rapid titration phase is complete, software application 1390 (e.g., algorithm) proceeds to maintenance titration phase 1438. Maintenance titration phase 1438 can be configured to apply first dosing regimen 1410 (e.g., basal insulin) for an extended period of time and monitor the user's reaction to the therapy. Maintenance titration phase 1438 can be further configured to account for large scale changes in the life of the user (e.g., adaptation to new diet and/or exercise, addition of new anti-diabetes medications, ongoing changes to insulin sensitivity). In some aspects, during maintenance titration phase 1438, software application 1390 can recommend increasing doses much slower than lowering doses. For example, a single day with excessive TBR may result in a recommendation to decrease the daily dose, but a pattern of multiple days of high MMG may be required for an increase in dose to be recommended). In some aspects, software application 1390 can monitor the user's reaction to the therapy during maintenance titration phase 1438 by determining one or more glucose metrics as discussed herein, and comparing the metrics to target levels or ranges. For example, the system may wirelessly track sensor glucose data over time (e.g., tracking average glucose, weekly average glucose, glucose TIR, etc.) and medication (dosing) data over time (e.g., tracking time between doses, time between last LA dose, no overnight treatments of hyperglycemia and/or hypoglycemia, sufficient overnight and/or morning data, sufficient time between last RA dose and morning glucose, etc.).

In some aspects, software application 1390 can implement maintenance titration phase 1438 for a period of time, for example, for a 14-day cycle. In some aspects, maintenance titration phase 1438 can be implemented for 12 days of dosing (e.g., basal insulin) over 14 days with sufficient glucose. In some aspects, maintenance titration phase 1438 can continue to be implemented, with no therapy changes or escalations being permitted, until the user exhibits sufficient glucose control (e.g., glucose metrics at or within a range of one or more targets) for a predetermined period of time, for example, for 12 days of dosing over 14 days with sufficient glucose control.

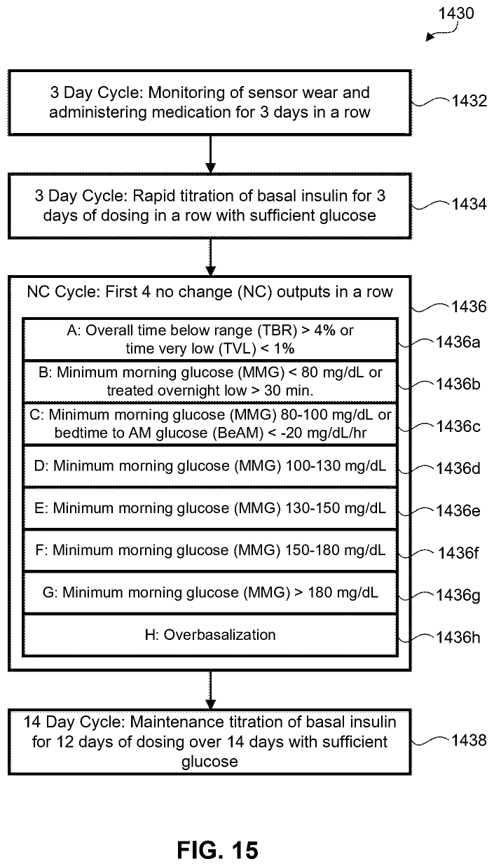

FIG. 16 illustrates second dosing regimen 1440, according to an exemplary aspect. Although second dosing regimen 1440 is shown in FIG. 16 as a stand-alone process, apparatus, and/or system, the aspects of this disclosure can be used with other processes, apparatuses, systems, and/or methods, such as, but not limited to, elements in FIGS. 13, 14A-14C, 15, 17, 18, 19A-19C, and 20-23, e.g., therapy management system 1300, software application 1390, control diagram 1400A, therapy assessment 1400B, control diagram 1400C, therapy assessment 1430, one or more dosing regimen 1410, 1450, 1460, 1480, flow diagram 2000, flow diagram 2100, flow diagram 2200, and/or computing device 2300.

The aspects of therapy assessment 1430 shown in FIG. 15, for example, and the aspects of second dosing regimen 1440 shown in FIG. 16 may be similar. Similar reference numbers are used to indicate features of the aspects of therapy assessment 1430 shown in FIG. 15 and the similar features of the aspects of second dosing regimen 1440 shown in FIG. 16. One difference between the aspects of therapy assessment 1430 shown in FIG. 15 and the aspects of second dosing regimen 1440 shown in FIG. 16 is that second dosing regimen 1440 includes a GLP-1 receptor antagonist or a dual GIP/GLP-1 receptor antagonist and rapid titration phase 1444 (e.g., 6 days of dosing over 7 days with sufficient glucose), rather than therapy assessment 1430 with basal insulin, rapid titration phase 1434, and no change phase 1436.

As shown in FIG. 16, second dosing regimen 1440 can include monitoring phase 1442, rapid titration phase 1444, and maintenance titration phase 1446. Monitoring phase 1442 can be configured to monitor one or more user compliance metrics of a current therapy over a predetermined period of time to ensure user adherence of the therapy. Rapid titration phase 1444 can be configured to introduce and apply a rapid dose of second dosing regimen 1440 (e.g., both GLP-1 receptor antagonist or dual GIP/GLP-1 receptor antagonist and LA insulin) and monitor the user's reaction to the additional therapy. Maintenance titration phase 1446 can be configured to apply second dosing regimen 1440 (e.g., both GLP-1 receptor antagonist or dual GIP/GLP-1 receptor antagonist and LA insulin) for an extended period of time and monitor the user's reaction to the therapy.

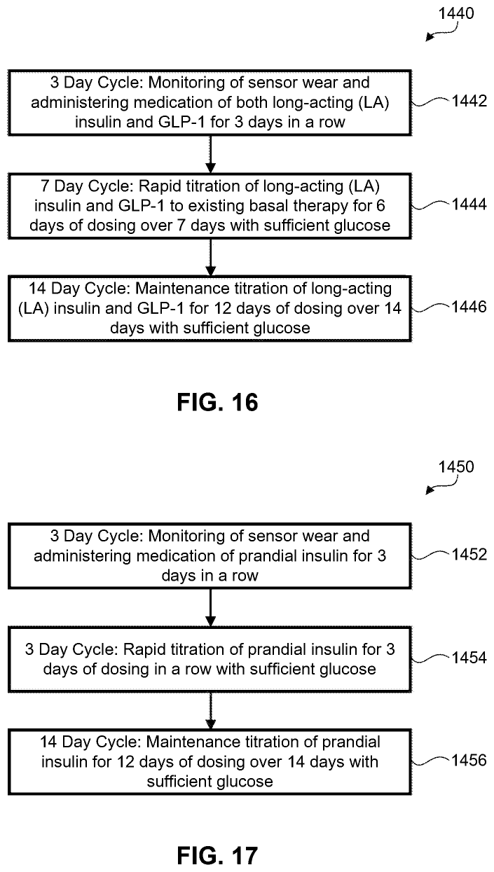

FIG. 17 illustrates third dosing regimen 1450, according to an exemplary aspect. Although third dosing regimen 1450 is shown in FIG. 17 as a stand-alone process, apparatus, and/or system, the aspects of this disclosure can be used with other processes, apparatuses, systems, and/or methods, such as, but not limited to, elements in FIGS. 13, 14A-14C, 15, 16, 18, 19A-19C, and 20-23, e.g., therapy management system 1300, software application 1390, control diagram 1400A, therapy assessment 1400B, control diagram 1400C, therapy assessment 1430, one or more dosing regimen 1410, 1440, 1460, 1480, flow diagram 2000, flow diagram 2100, flow diagram 2200, and/or computing device 2300.

The aspects of therapy assessment 1430 shown in FIG. 15, for example, and the aspects of third dosing regimen 1450 shown in FIG. 17 may be similar. Similar reference numbers are used to indicate features of the aspects of therapy assessment 1430 shown in FIG. 15 and the similar features of the aspects of third dosing regimen 1450 shown in FIG. 17. One difference between the aspects of therapy assessment 1430 shown in FIG. 15 and the aspects of third dosing regimen 1450 shown in FIG. 17 is that third dosing regimen 1450 includes prandial insulin with one meal, rather than therapy assessment 1430 with basal insulin and no change phase 1436.

As shown in FIG. 17, third dosing regimen 1450 can include monitoring phase 1452, rapid titration phase 1454, and maintenance titration phase 1456. Monitoring phase 1452 can be configured to monitor one or more user compliance metrics of a current therapy over a predetermined period of time to ensure user adherence of the therapy. Rapid titration phase 1454 can be configured to introduce and apply a rapid dose of third dosing regimen 1450 (e.g., prandial insulin with one meal) and monitor the user's reaction to the additional therapy. Maintenance titration phase 1456 can be configured to apply third dosing regimen 1450 (e.g., prandial insulin with one meal) for an extended period of time and monitor the user's reaction to the therapy.

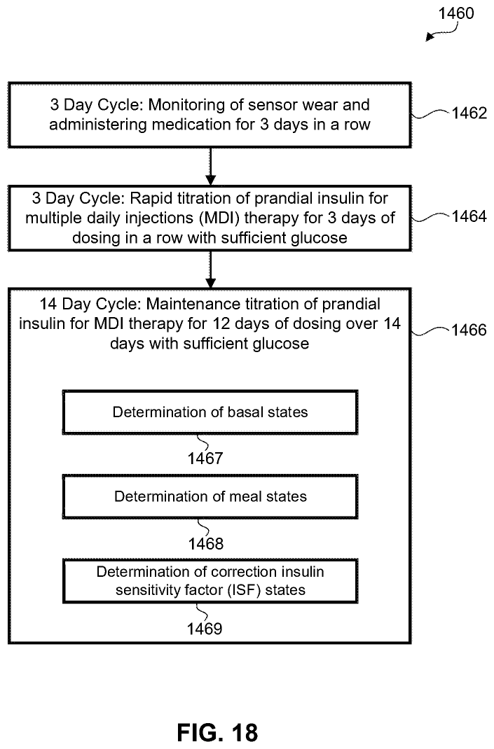

FIG. 18 illustrates fourth dosing regimen 1460, according to an exemplary aspect. Although fourth dosing regimen 1460 is shown in FIG. 18 as a stand-alone process, apparatus, and/or system, the aspects of this disclosure can be used with other processes, apparatuses, systems, and/or methods, such as, but not limited to, elements in FIGS. 13, 14A-14C, 15-17, 19A-19C, and 20-23, e.g., therapy management system 1300, software application 1390, control diagram 1400A, therapy assessment 1400B, control diagram 1400C, therapy assessment 1430, one or more dosing regimen 1410, 1440, 1450, 1480, flow diagram 2000, flow diagram 2100, flow diagram 2200, and/or computing device 2300.

The aspects of therapy assessment 1430 shown in FIG. 15, for example, and the aspects of fourth dosing regimen 1460 shown in FIG. 18 may be similar. Similar reference numbers are used to indicate features of the aspects of therapy assessment 1430 shown in FIG. 15 and the similar features of the aspects of fourth dosing regimen 1460 shown in FIG. 18. One difference between the aspects of therapy assessment 1430 shown in FIG. 15 and the aspects of fourth dosing regimen 1460 shown in FIG. 18 is that fourth dosing regimen 1460 includes stepwise MDI of prandial insulin with a plurality of meals and maintenance titration phase 1466 that includes basal assessment 1467, meal assessment 1468, and correction ISF assessment 1469, rather than therapy assessment 1430 with basal insulin, no change phase 1436 with basal states 1436a-1436h, and maintenance titration phase 1438.

As shown in FIG. 18, fourth dosing regimen 1460 can include monitoring phase 1462, rapid titration phase 1464, and maintenance titration phase 1466. Monitoring phase 1462 can be configured to monitor one or more user compliance metrics of a current therapy over a predetermined period of time to ensure user adherence of the therapy. Rapid titration phase 1464 can be configured to introduce and apply a rapid dose of fourth dosing regimen 1460 (e.g., stepwise MDI of prandial insulin with a plurality of meals) and monitor the user's reaction to the additional therapy. Maintenance titration phase 1466 can be configured to apply fourth dosing regimen 1460 (e.g., stepwise MDI of prandial insulin with a plurality of meals) for an extended period of time and monitor the user's reaction to the therapy.

As shown in FIG. 18, maintenance titration phase 1466 can include basal assessment 1467, meal assessment 1468, and/or correction ISF assessment 1469. In some aspects, basal assessment 1467, meal assessment 1468, and/or correction ISF assessment 1469 can be used with one or more dosing regimen (e.g., control diagram 1400A) to determine overbasalization and/or monitor for any changes or side-effects of the current therapy.

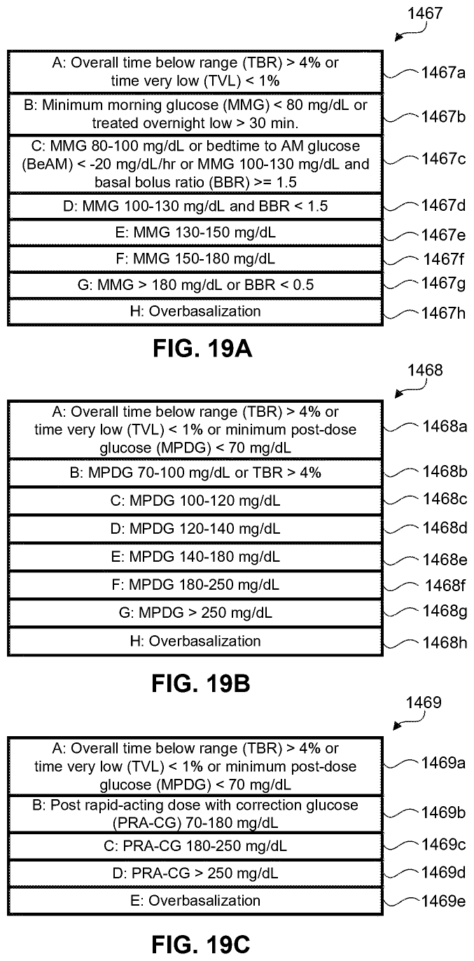

Basal assessment 1467 can be configured to monitor any changes to one or more basal states (e.g., basal states 1467a-1467h shown in FIG. 19A), for example, during maintenance titration phase 1466. Basal assessment 1467 can be further configured to determine overbasalization and any changes or side-effects of the current therapy. Basal assessment 1467 can be further configured to assist software application 1390 in determining any trends (e.g., increasing, decreasing, no change) of basal states over time and whether any meal action and/or correction action should be recommended.

Meal assessment 1468 can be configured to monitor any changes to one or more meal states (e.g., meal states 1468a-1468h shown in FIG. 19B), for example, during maintenance titration phase 1466. Meal assessment 1468 can be further configured to determine overbasalization and any changes or side-effects of the current therapy. Meal assessment 1468 can be further configured to assist software application 1390 in determining any trends (e.g., increasing, decreasing, no change) of meal states over time and whether any basal action and/or correction action should be recommended.

Correction ISF assessment 1469 can be configured to monitor any changes to one or more correction ISF states (e.g., correction ISF states 1469a-1469e shown in FIG. 19C), for example, during maintenance titration phase 1466. Correction ISF assessment 1469 can be further configured to determine overbasalization and any changes or side-effects of the current therapy. Correction ISF assessment 1469 can be further configured to assist software application 1390 in determining any trends (e.g., increasing, decreasing, no change) of correction ISF states over time and whether any basal action and/or meal action should be recommended.

FIG. 19A illustrates basal assessment 1467, according to an exemplary aspect. Although basal assessment 1467 is shown in FIG. 19A as a stand-alone process, apparatus, and/or system, the aspects of this disclosure can be used with other processes, apparatuses, systems, and/or methods, such as, but not limited to, elements in FIGS. 13, 14A-14C, 15-18, and 20-23, e.g., therapy management system 1300, software application 1390, control diagram 1400A, therapy assessment 1400B, control diagram 1400C, therapy assessment 1430, one or more dosing regimen 1410, 1440, 1450, 1460, 1480, flow diagram 2000, flow diagram 2100, flow diagram 2200, and/or computing device 2300.

As shown in FIG. 19A, basal assessment 1467 can include monitoring one or more basal states 1467a-1467h. In some aspects, one or more basal states 1467a-1467h can include first basal state 1467a (e.g., excessively below target), second basal state 1467b (e.g., significantly below target), third basal state 1467c (e.g., below target), fourth basal state 1467d (e.g., target), fifth basal state 1467e (e.g., above target), sixth basal state 1467f (e.g., significantly above target), seventh basal state 1467g (e.g., excessively above target, saturation), eighth basal state 1467h (e.g., overbasalization), or a combination thereof.

In some aspects, first basal state 1467a can be determined based on an overall glucose TBR of greater than about 4% or glucose TVL of less than about 1%. In some aspects, second basal state 1467b can be determined based on a MMG of less than about 80 mg/dL or a treated overnight low greater than about 30 minutes. In some aspects, third basal state 1467c can be determined based on a MMG of about 80 mg/dL to about 100 mg/dL or a BeAM of less than about −20 mg/dL/hr, or a MMG of about 100 mg/dL to about 130 mg/dL and a BBR of greater than or equal to about 1.5. In some aspects, fourth basal state 1467d can be determined based on a MMG of about 100 mg/dL to about 130 mg/dL and a BBR of less than about 1.5. In some aspects, fifth basal state 1467e can be determined based on a MMG of about 130 mg/dL to about 150 mg/dL. In some aspects, sixth basal state 1467f can be determined based on a MMG of about 150 mg/dL to about 180 mg/dL. In some aspects, seventh basal state 1467g can be determined based on a MMG of greater than about 180 mg/dL or a BBR of less than about 0.5. In some aspects, eighth basal state 1467h can be determined by therapy assessment 1430 or by one or more overbasalization metrics described above (e.g., ratio of TDD to body weight, glucose variability, hypoglycemia metric, change in glucose in meal period, change in glucose in overnight period).

FIG. 19B illustrates meal assessment 1468, according to an exemplary aspect. Although meal assessment 1468 is shown in FIG. 19B as a stand-alone process, apparatus, and/or system, the aspects of this disclosure can be used with other processes, apparatuses, systems, and/or methods, such as, but not limited to, elements in FIGS. 13, 14A-14C, 15-18, and 20-23, e.g., therapy management system 1300, software application 1390, control diagram 1400A, therapy assessment 1400B, control diagram 1400C, therapy assessment 1430, one or more dosing regimen 1410, 1440, 1450, 1460, 1480, flow diagram 2000, flow diagram 2100, flow diagram 2200, and/or computing device 2300.

As shown in FIG. 19B, meal assessment 1468 can include monitoring one or more meal states 1468a-1468h. In some aspects, one or more meal states 1468a-1468h can include first meal state 1468a (e.g., significantly below target), second meal state 1468b (e.g., below target), third meal state 1468c (e.g., target), fourth meal state 1468d (e.g., above target), fifth meal state 1468e (e.g., significantly above target), sixth meal state 1468f (e.g., excessively above target), seventh meal state 1468g (e.g., saturation), eighth meal state 1468h (e.g., overbasalization), or a combination thereof.

In some aspects, first meal state 1468a can be determined based on an overall glucose TBR of greater than about 4% or glucose TVL of less than about 1%, or a MPDG of less than about 70 mg/dL. In some aspects, second meal state 1468b can be determined based on a MPDG of about 70 mg/dL to about 100 mg/dL or a glucose TBR of greater than about 4%. In some aspects, third meal state 1468c can be determined based on a MPDG of about 100 mg/dL to about 120 mg/dL. In some aspects, fourth meal state 1468d can be determined based on a MPDG of about 120 mg/dL to about 140 mg/dL. In some aspects, fifth meal state 1468e can be determined based on a MPDG of about 140 mg/dL to about 180 mg/dL. In some aspects, sixth meal state 1468f can be determined based on a MPDG of about 180 mg/dL to about 250 mg/dL. In some aspects, seventh meal state 1468g can be determined based on a MPDG of greater than about 250 mg/dL. In some aspects, eighth meal state 1468h can be determined by therapy assessment 1430 or by one or more overbasalization metrics described above (e.g., ratio of TDD to body weight, glucose variability, hypoglycemia metric, change in glucose in meal period, change in glucose in overnight period).

FIG. 19C illustrates correction ISF assessment 1469, according to an exemplary aspect. Although correction ISF assessment 1469 is shown in FIG. 19C as a stand-alone process, apparatus, and/or system, the aspects of this disclosure can be used with other processes, apparatuses, systems, and/or methods, such as, but not limited to, elements in FIGS. 13, 14A-14C, 15-18, and 20-23, e.g., therapy management system 1300, software application 1390, control diagram 1400A, therapy assessment 1400B, control diagram 1400C, therapy assessment 1430, one or more dosing regimen 1410, 1440, 1450, 1460, 1480, flow diagram 2000, flow diagram 2100, flow diagram 2200, and/or computing device 2300.

As shown in FIG. 19C, correction ISF assessment 1469 can include monitoring one or more correction ISF states 1469a-1469e. In some aspects, one or more correction ISF states 1469a-1469e can include first correction ISF state 1469a (e.g., below target), second correction ISF state 1469b (e.g., target), third correction ISF state 1469c (e.g., above target), fourth correction ISF state 1469d (e.g., significantly above target), fifth correction ISF state 1469e (e.g., overbasalization), or a combination thereof.

In some aspects, first correction ISF state 1469a can be determined based on an overall glucose TBR of greater than about 4% or glucose TVL of less than about 1%, or a MPDG of less than about 70 mg/dL. In some aspects, second correction ISF state 1469b can be determined based on a PRA-CG of about 70 mg/dL to about 180 mg/dL. In some aspects, third correction ISF state 1469c can be determined based on a PRA-CG of about 180 mg/dL to about 250 mg/dL. In some aspects, fourth correction ISF state 1469d can be determined based on a PRA-CG of greater than about 250 mg/dL. In some aspects, fifth correction ISF state 1469e can be determined by therapy assessment 1430 or by one or more overbasalization metrics described above (e.g., ratio of TDD to body weight, glucose variability, hypoglycemia metric, change in glucose in meal period, change in glucose in overnight period).

### Exemplary Flow Diagram

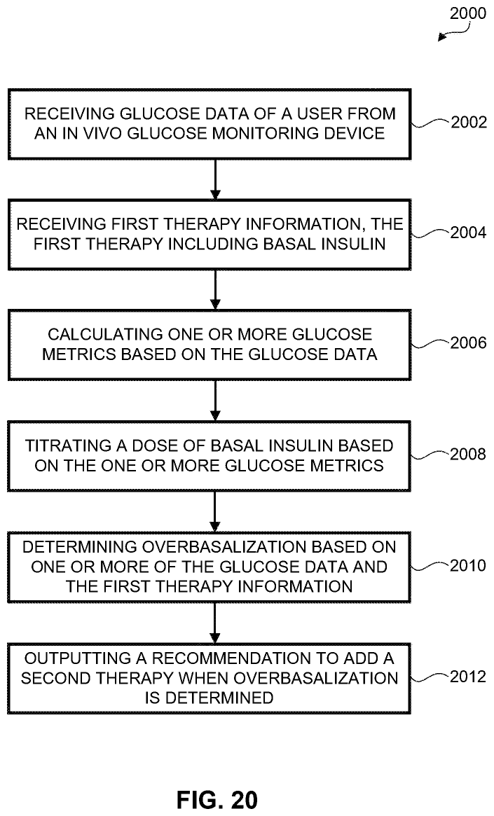

FIG. 20 illustrates flow diagram 2000 according to an exemplary aspect. For example, flow diagram 2000 can be for therapy management system 1300 shown in FIG. 13. Flow diagram 2000 can be configured to measure one or more metrics (e.g., glucose metrics, therapy metrics) of a first therapy (e.g., basal insulin) and titrate a dose of the first therapy based at least in part on the one or more metrics. Flow diagram 2000 can be further configured to determine overbasalization of the first therapy based on the one or more metrics. Flow diagram 2000 can be further configured to output a recommendation to add a second therapy (e.g., insulin analog, GLP-1, prandial insulin, MDI of prandial insulin, etc.) if overbasalization is determined or if one or more user metrics remains outside of one or more targets (e.g., one or more glucose metrics, A1C level, body weight).

It is to be appreciated that not all steps in FIG. 20 are needed to perform the disclosure provided herein. Further, some of the steps may be performed simultaneously, sequentially, and/or in a different order than shown in FIG. 20. Flow diagram 2000 shall be described with reference to FIGS. 13, 14A-14C, 15-18, 19A-19C, and 23. However, flow diagram 2000 is not limited to those example aspects. Although flow diagram 2000 is shown in FIG. 20 as a stand-alone method, the aspects of this disclosure can be used with other processes, apparatuses, systems, and/or methods, such as, but not limited to, elements in FIGS. 13, 14A-14C, 15-18, 19A-19C, and 21-23, e.g., control diagram 1400A, therapy assessment 1400B, control diagram 1400C, therapy assessment 1430, one or more dosing regimen 1410, 1440, 1450, 1460, 1480, flow diagram 2100, flow diagram 2200, and/or computing device 2300. In some aspects, flow diagram 2000 can be implemented by software application 1390. In some aspects, flow diagram 2000 can be implemented by one or more models or algorithms run on one or more processors and/or computing devices based on one or more instructions stored in one or more memories.

In step 2002, as shown in the example of FIGS. 13-18, 19A-19C, and 23, glucose data of a user can be received from an in vivo glucose monitoring device. In some aspects, glucose data can be received from OBU 1350 of therapy management system 1300 (e.g., via software application 1390).

In step 2004, as shown in the example of FIGS. 13-18, 19A-19C, and 23, first therapy information can be received. In some aspects, first therapy can include basal insulin. In some aspects, first therapy information can be received from a medication delivery device (e.g., first injection pen 1360, second injection pen 1370, insulin pump, smart pill bottle, etc.) of therapy management system 1300 (e.g., via software application 1390). In some aspects, first therapy information can include one or more user metrics, including but not limited to, user input, user feedback, data from the medication delivery device, a body weight measurement, applied dose, TDD, ratio of TDD to body weight, time between doses, type of medication, side-effects of first therapy, BMI, heart rate, blood pressure, meal data, exercise data, or a combination thereof.

In step 2006, as shown in the example of FIGS. 13-18, 19A-19C, and 23, one or more glucose metrics can be calculated based on the glucose data. In some aspects, software application 1390 of therapy management system 1300 can receive the glucose data and calculate the one or more glucose metrics. In some aspects, the one or more glucose metrics can include average glucose, glucose TIR, glucose TBR, glucose TVL, GMI, MMG, MPDG, PPG, BeAM, a change in glucose level in a meal period or an overnight period, a post-meal glucose rise for one or more meals, or a combination thereof.

In step 2008, as shown in the example of FIGS. 13-18, 19A-19C, and 23, a dose of basal insulin can be titrated based on the one or more glucose metrics. In some aspects, software application 1390 of therapy management system 1300 can titrate the dose of basal insulin based on the one or more glucose metrics.

In step 2010, as shown in the example of FIGS. 13-18, 19A-19C, and 23, overbasalization of first therapy can be determined based on one or more of the glucose data and the first therapy information. In some aspects, software application 1390 of therapy management system 1300 can determine whether overbasalization has occurred based on one or more of the glucose data and the first therapy information. In some aspects, software application 1390 can determine overbasalization based at least in part on a ratio of TDD of insulin to a body weight of the user, glucose variability, a hypoglycemia metric, a change in glucose level in a meal period or an overnight period, or a combination thereof.

In step 2012, as shown in the example of FIGS. 13-18, 19A-19C, and 23, a recommendation to add a second therapy can be outputted when overbasalization is determined. In some aspects, software application 1390 of therapy management system 1300 can output a recommendation (e.g., via remote device 1310) to the user or a HCP to add or initiate the second therapy. In some aspects, the second therapy can include basal analogs, a GLP-1 receptor agonist, a dual GIP/GLP-1 receptor agonist, prandial insulin, MDI of prandial insulin, or a combination thereof.

In some aspects, flow diagram 2000 can further include recommending decreasing the basal insulin dose when the second therapy is added. In some aspects, flow diagram 2000 can further include titrating a dose of the second therapy. In some aspects, for example, titrating the dose of the second therapy can include receiving a user input (e.g., feedback) regarding any side-effects of the second therapy and recommending an increased dose when the user input indicates no side-effects, for example, for a monitoring period (e.g., 14 days). In some aspects, for example, titrating the dose of the second therapy can include receiving a body weight measurement of the user (e.g., via a weight scale in communication with software application 1390) and recommending an increased dose when the body weight measurement is above a target body weight (e.g., 80 kg).

In some aspects, flow diagram 2000 can further include recommending initiating a prandial (bolus) insulin dose for a first meal when a post-meal glucose rise for one meal exceeds a threshold. In some aspects, flow diagram 2000 can further include recommending initiating a prandial (bolus) insulin dose for a plurality of meals (e.g., MDI) when the post-meal glucose rise for the plurality of meals exceeds a threshold.

### Exemplary Non-Adherence Detection

Users may not comply, intentionally or unintentionally with the dose guidance system and its recommendations. The titration algorithm is not effective if the user is not taking the recommended doses, such as skipping doses or taking other dose amounts. Further, if the system recommends a dose and the user's glucose levels remain high because of non-adherence, the system may continue to recommend higher and higher doses. If the user then decides to take a dose after a period of non-compliance, the dose may be too high and may be unsafe.

It would be beneficial to detect if the user is not complying with the dose guidance, and to encourage the user to start taking the recommended doses or otherwise to provide guidance to the user to education them and improve therapy compliance.

Some embodiments described herein relate to determining non-adherence to a therapy recommendation. The system may determine non-adherence based on analysis of glucose metrics before and after a dose recommendation. The system may determine non-adherence based on determining a glucose metric before and after the dose recommendation and determining if there is a change in the glucose metric. The system may also determine an expected change and compare the actual change to the expected change to determine adherence.

The system may determine a minimum glucose metric in a first time period preceding a dose recommendation, and may determine a minimum glucose metric in a second time period following the dose recommendation. The minimum glucose metric may be based on a fasting glucose level. The system may determine non-adherence if the change in the minimum glucose metric is less than a threshold change in the minimum glucose metric. This is because administration of the recommended dose is expected to result in a change in the minimum glucose metric. Thus, no change or minimal change may indicate that the user did not administer the recommended dose. The system may detect if an increased dose recommendation resulted in a drop in glucose levels or in a glucose metric. Alternatively, the system may detect if a decreased dose recommendation resulted in an increase in glucose levels or in a glucose metric. A change in the expected direction may be sufficient confirmation of adherence to the therapy, whereas if the glucose levels increase despite an increasing dose recommendation, that may indicate non-adherence. The system may predict an expected range of glucose levels or an expected range for the glucose metric for a given dose recommendation. The system may determine if the actual glucose levels following the dose recommendation are within the predicted range of values to determine adherence. Upon detecting non-adherence, the system may initiate one or more remedial actions.

The minimum glucose metric may be a minimum morning glucose (MMG). A longest fasting period is typically an overnight period when the user is sleeping. Thus, the MMG may be used as a proxy for fasting glucose by taking a glucose level in a morning window, such as from 4 AM-8 AM, however other windows may be used. The use of MMG may require the system to have more information about the user, such as to confirm when the user is awake or sleeping to ensure the morning glucose level is a good proxy for fasting glucose levels. Further, MMG may be a poor proxy for fasting glucose when the user is not on a typical schedule, such as shift workers who work overnight, or when the user is traveling to different time zones, or otherwise maintains an irregular schedule. Another metric is the daily minimum hourly average glucose (DMHAG). The average glucose level may be computed for each hour of the day, and the daily minimum hourly average may be the lowest average glucose level during the day. This metric may be beneficial as it does not require identification of the fasting period, or that the fasting period occur at a particular time of day. However, DMHAG is only applicable for patients on basal insulin only therapy regimens, as the rapid-acting insulin doses taken at meals would influence the low glucose levels in addition to the basal insulin.

A system as described below can detect non-adherence of a user to dose recommendations based on glucose data. In some aspects, the system can receive glucose data before and after an administered dose to determine whether the user is adhering to a dose regimen. In some aspects, the system can calculate one or more glucose metrics before and after an administered dose. In some aspects, the system can calculate one or more minimum glucose metrics before and after an administered dose. In some aspects, for example, the minimum glucose metric can be MMG. In some aspects, for example, the minimum glucose metric can be DMHAG.

In some aspects, the system can determine adherence or non-adherence of the user to the dose regimen (e.g., a recommended dose) based on whether the one or more minimum glucose metrics changes by a predetermined amount (e.g., MMG outside a range of a predicted level). In some aspects, the system can determine adherence or non-adherence of the user to the dose regimen (e.g., a recommended dose) based on whether the one or more glucose metrics changes in a correct direction. For example, for an increased dose amount, adherence can be determined if there is a measured drop in glucose levels. In some aspects, the system can determine adherence or non-adherence of the user by predicting what the glucose level and/or the one or more glucose metrics should be after an administered dose and whether measured glucose data and/or one or more glucose metrics is outside the predicted range. In some aspects, the system can determine adherence or non-adherence of the user based on historic glucose data to determine glucose level changes per administered dose and/or one or more glucose metrics changes per administered dose. In some aspects, for example, the system can predict and/or extrapolate an impact of a unit dose change based on the determined glucose level changes per administered dose and/or one or more glucose metrics changes per administered dose.

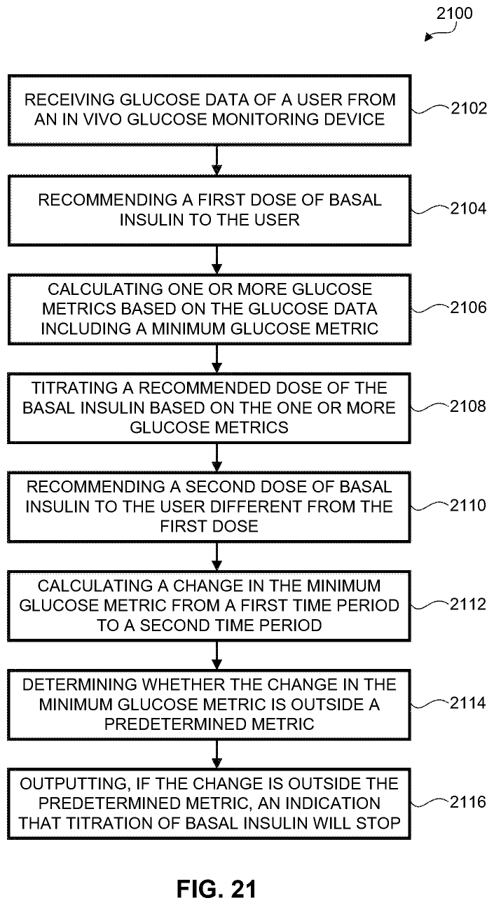

FIG. 21 illustrates flow diagram 2100 according to an exemplary aspect. For example, flow diagram 2100 can be for therapy management system 1300 shown in FIG. 13. Flow diagram 2100 can be configured to measure one or more metrics (e.g., minimum glucose metrics) of a first therapy (e.g., basal insulin) and titrate a dose of the first therapy based at least in part on the one or more metrics. Flow diagram 2100 can be further configured to determine non-adherence to one or more dose recommendations by the user, for example, non-adherence to one or more basal insulin dose recommendations. Flow diagram 2100 can be further configured to output an indication or notification (e.g., to the user, to a third party, etc.) that titration of basal insulin will stop if a change in the minimum glucose metric (e.g., fasting glucose, daily minimum glucose (DMG), minimum morning glucose (MMG), daily minimum hourly average glucose (DMHAG), etc.) is outside a predetermined metric.

It is to be appreciated that not all steps in FIG. 21 are needed to perform the disclosure provided herein. Further, some of the steps may be performed simultaneously, sequentially, and/or in a different order than shown in FIG. 21. Flow diagram 2100 shall be described with reference to FIGS. 13, 14A-14C, 15-18, 19A-19C, and 23. However, flow diagram 2100 is not limited to those example aspects. Although flow diagram 2100 is shown in FIG. 21 as a stand-alone method, the aspects of this disclosure can be used with other processes, apparatuses, systems, and/or methods, such as, but not limited to, elements in FIGS. 13, 14A-14C, 15-18, 19A-19C, 20, 22, and 23, e.g., control diagram 1400A, therapy assessment 1400B, control diagram 1400C, therapy assessment 1430, one or more dosing regimen 1410, 1440, 1450, 1460, 1480, flow diagram 2000, flow diagram 2200, and/or computing device 2300. In some aspects, flow diagram 2100 can be implemented by software application 1390. In some aspects, flow diagram 2100 can be implemented by one or more models or algorithms run on one or more processors and/or computing devices based on one or more instructions stored in one or more memories.

In step 2102, as shown in the example of FIGS. 13-18, 19A-19C, and 23, glucose data of a user can be received from an in vivo glucose monitoring device. In some aspects, glucose data can be received from OBU 1350 of therapy management system 1300 (e.g., via software application 1390).

In step 2104, as shown in the example of FIGS. 13-18, 19A-19C, and 23, a first dose of basal insulin can be recommended.

In step 2106, as shown in the example of FIGS. 13-18, 19A-19C, and 23, one or more glucose metrics can be calculated based on the glucose data. The one or more glucose metrics can include a minimum glucose metric. In some aspects, software application 1390 of therapy management system 1300 can receive the glucose data and calculate the one or more glucose metrics. For example, the one or more glucose metrics can be calculated in the period following the first dose recommendation. In some aspects, the one or more glucose metrics can include average glucose, glucose TIR, glucose TBR, glucose TVL, GMI, MMG, MPDG, PPG, BeAM, a change in glucose level in a meal period or an overnight period, a post-meal glucose rise for one or more meals, or a combination thereof. In some aspects, the minimum glucose metric can include fasting glucose, daily minimum glucose (DMG), minimum morning glucose (MMG), average glucose for a predetermined time period, daily minimum hourly average glucose (DMHAG), or a combination thereof.

In some aspects, one or more minimum glucose metrics (e.g., fasting glucose, DMG, MMG, DMHAG, etc.) can be calculated based on the glucose data. The one or more minimum glucose metrics can be preferred over other glucose metrics to provide a more accurate indicator of the glucose level of a user. For example, MMG can be used as a proxy for fasting glucose and be based on a glucose level in a morning window, which is typically at the end of a longest fasting period of an overnight period when the user is sleeping, that can provide a more accurate and safer indicator of the fasting glucose level of the user since it is measured after the longest fasting period.

In some aspects, the one or more minimum glucose metrics can be based on DMHAG. For example, DMHAG can be used as a proxy for fasting glucose and be based on an hour of the day having the lowest average glucose, which can be determined irrespective of a fasting period or a particular time of day, that can provide an alternative indicator of the fasting glucose level of the user. In some aspects, DMHAG can be used as a safer alternative than MMG since DMHAG has similar accuracy to MMG but does not require meal and dosing data. For example, DMHAG can be used for a user on an irregular schedule, for example, shift workers who work overnight, travelers in different time zones, or any other user that maintains an irregular schedule.

In step 2108, as shown in the example of FIGS. 13-18, 19A-19C, and 23, a recommended dose of the basal insulin can be titrated based on the one or more glucose metrics. In some aspects, software application 1390 of therapy management system 1300 can titrate the recommended dose of basal insulin based on the one or more glucose metrics. In some aspects, flow diagram 2100 does not require basal insulin dose amount data and can titrate the recommended dose (e.g., via software application 1390) based on the one or more glucose metrics (e.g., via OBU 1350) and/or basal insulin dose time data (e.g., via first smart cap 1382 and/or second smart cap 1384).

In step 2110, as shown in the example of FIGS. 13-18, 19A-19C, and 23, a second dose of basal insulin can be recommended. In some aspects, the second dose can be different from the first dose, for example, due to titrating the recommended dose.

In step 2112, as shown in the example of FIGS. 13-18, 19A-19C, and 23, a change in the minimum glucose metric can be calculated. In some aspects, the change in the minimum glucose metric can be calculated from a first time period to a second time period. In some aspects, for example, the change can be calculated from a first time period prior to recommending the second dose to a second time period after recommending the second dose. In some aspects, software application 1390 of therapy management system 1300 can receive the glucose data and calculate a change in the minimum glucose metric. In some aspects, the change in the minimum glucose metric can include a change in fasting glucose, DMG, MMG, average glucose for a predetermined time period, DMHAG, or a combination thereof.

In step 2114, as shown in the example of FIGS. 13-18, 19A-19C, and 23, whether the change in the minimum glucose metric is outside a predetermined metric can be determined. In some aspects, software application 1390 of therapy management system 1300 can determine whether the change in the minimum glucose metric is outside a predetermined metric. In some aspects, the predetermined metric can include the change in the minimum glucose metric being less than 5 mg/dL, less than 10 mg/dL, or less than 15 mg/dL, among other amounts. In some aspects, the predetermined metric can be based on which minimum glucose metric is utilized. For example, the predetermined metric for MMG can include a change in MMG being less than 5 mg/dL, less than 10 mg/dL, or less than 15 mg/dL, among other amounts. In some aspects, the predetermined metric for DMHAG can include a change in DMHAG being less than 0.1 mg/dL, less than 1 mg/dL, less than 5 mg/dL, less than 10 mg/dL, among other amounts.

In some aspects, the predetermined metric can include the change in the minimum glucose metric being less than a predetermined percentage of an expected change of the minimum glucose metric. The predetermined percentage may be 50% of an expected change of the minimum glucose metric. In some aspects, the expected change of the minimum glucose metric can be based on one or more user metrics. In some aspects, the one or more user metrics can include body weight, BMI, age, ISF, TDD, ratio of TDD to body weight, A1C level, heart rate, blood pressure, or a combination thereof. A patient with a higher insulin sensitivity would exhibit a smaller change in glucose levels for a given dose of insulin relative to a patient with a lower insulin sensitivity. Further, as weight may be a proxy for insulin sensitivity, increasing the dose by 4 U would be expected to have a smaller impact on glucose levels of a patient having a greater weight than a 4 U dose administered to a person having a relatively low weight. In some aspects, the expected change of the minimum glucose metric can be based on historical glucose data of the user. For example, if a previous increase in dose by 1 U resulted in a 10 mg/dl decrease in glucose levels, the system may predict or expect that a further increase by 1 U would result in a further 10 mg/dL decrease in glucose levels. Further, the system may determine a trend or extrapolate based on historical data. For example, if a 1 U dose previously resulted in a 10 mg/dL decrease in glucose levels, a 2 U dose may be expected to result in a 20 mg/dL decrease. In some aspects, the expected change of the minimum glucose metric can be proportional to a change between the first dose and the second dose.

In some aspects, the predetermined metric can include a statistical test of the minimum glucose metric that does not support a hypothesis that the minimum glucose metric has changed. In some aspects, the statistical test can include an insulin sensitivity test (IST), an insulin tolerance test (ITT), an oral glucose tolerance test (OGTT), a fasting plasma glucose (FPG) test, a random plasma glucose test, or a combination thereof.

In step 2116, as shown in the example of FIGS. 13-18, 19A-19C, and 23, an indication that titration of basal insulin with stop can be outputted if the change in the minimum glucose metric is outside the predetermined metric. In some aspects, software application 1390 of therapy management system 1300 can output an indication (e.g., via remote device 1310) to the user or a HCP (e.g., third party) that titration of basal insulin will stop (e.g., to avoid a dangerous dosing event, hypoglycemia event, hyperglycemia event).

In some aspects, flow diagram 2100 can output a notification to the user. The notification may indicate that the user has not taken the recommended dose. The notification may encourage the user to administer the recommended dose. The notification may prompt the user to confirm administration of the second dose. In some aspects, flow diagram 2100 can further include resuming, if the user confirms administration of the second dose, dose guidance to the user. In some aspects, flow diagram 2100 can further include recommending, if the user does not confirm administration of the second dose, a third dose based on titrating the second dose. The notification may provide educational materials or direct the user to educational materials regarding their therapy and/or operation of the dose guidance system.

In some aspects, flow diagram 900 can further include outputting a prompt to the user to confirm the user is following the recommended dose. In some aspects, flow diagram 900 can further include outputting a prompt to the user to have the user seek guidance from a health care professional. In some aspects, flow diagram 900 can further include outputting a quiz to the user to verify the user is following the recommended dose. In some aspects, for example, software application 190 of therapy management system 100 can output an quiz (e.g., via remote device 110) to the user (e.g., “What is the current basal insulin dose amount?”, “What was the last basal insulin dose amount?”, “What is the recommended basal insulin dose amount?”, etc.).The titration may continue if the user answers correctly. If the user enters a different dose than was recommended, the titration algorithm may proceed based on the user entered dose as the dose amount for titration.

In some aspects, flow diagram 2100 can further include recommending limiting a change in dose of basal insulin if there is no change in the minimum glucose metric after titrating the recommended dose for a predetermined time period. In some aspects, the predetermined time period can include at least 3 days. In some aspects, the predetermined time period can include at least 5 days. In some aspects, the predetermined time period can include at least 7 days. In some aspects, the predetermined time period can include a range of 1 day to 14 days.

In some aspects, flow diagram 2100 can further include recommending stopping upward titration (i.e., increasing the dose amount) of basal insulin if the minimum glucose metric (e.g., fasting glucose, MMG, DMHAG, etc.) does not decrease after titrating the recommended dose for a predetermined time period. In some aspects, flow diagram 2100 can further include recommending stopping downward titration (i.e., decreasing the dose amount) of basal insulin if the minimum glucose metric (e.g., fasting glucose, MMG, DMHAG, etc.) does not increase after titrating the recommended dose for a predetermined time period.

In some aspects, flow diagram 2100 can further include activating a blinded mode such that glucose data is not displayed to the user. In some aspects, the blinded mode can prevent display of glucose data to the user to encourage adherence by the user to the dose regimen and decrease improper dosing and/or interaction by the user to correct their glucose levels (e.g., prevent the user from deviating outside the recommended dose).

In some aspects, flow diagram 2100 can further include initiating, if the change in the minimum glucose metric is outside the predetermined metric, a counter configured to monitor a number of titration cycles. In some aspects, flow diagram 2100 can further include recommending stopping titration of basal insulin if the change in the minimum glucose metric remains outside the predetermined metric after a predetermined number of titration cycles with increasing or decreasing doses of basal insulin.

In some aspects, flow diagram 2100 can further include receiving basal insulin dose administration time data. In some aspects, basal insulin dose administration time data can be determined via software application 1390 based on a change in glucose levels (e.g., received from OBU 1350) and/or received from a medication delivery device and/or pen cap (e.g., first smart cap 1382, second smart cap 1384) of therapy management system 1300 (e.g., via software application 1390). In some aspects, the basal insulin dose administration time data can supplement one or more glucose metrics in determining a recommended dose.

In some aspects, flow diagram 2100 can detect non-adherence to a recommended basal insulin dose. In some aspects, flow diagram 2100 can prevent an unsafe continual increase or decrease of a recommended dose that may lead to an unsafe basal insulin dose. In some aspects, flow diagram 2100 can include one or more outputs (e.g., indication, notification, prompt, etc.) to the user to have the user confirm they are following the dose recommendations, to seek guidance from their HCP, and/or other verification means (e.g., quiz, query, etc.) to confirm the user is following the dose recommendations.

In some aspects, the non-adherence determination may be applied to rapid-acting doses for a meal. However, the glucose metric would be based on a glucose metric in the post-prandial period following the meal dose rather than MMG or DMHAG. The glucose metric for the post-prandial period for the meal prior to the new dose recommendation and after the dose recommendation can be compared to determine a change in the metric.

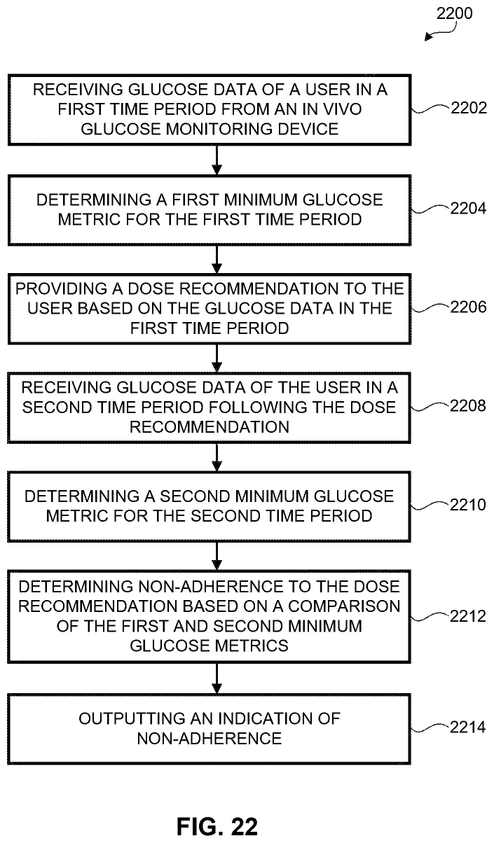

FIG. 22 illustrates flow diagram 2200 according to an exemplary aspect. For example, flow diagram 2200 can be for therapy management system 1300 shown in FIG. 13. Flow diagram 2200 can be configured to measure one or more metrics (e.g., minimum glucose metrics) of a therapy (e.g., basal insulin) to determine non-adherence to one or more dose recommendations by the user. Flow diagram 2200 can be further configured to output an indication or notification (e.g., to the user, to a third party, etc.) of non-adherence to a dose recommendation by the user.

It is to be appreciated that not all steps in FIG. 22 are needed to perform the disclosure provided herein. Further, some of the steps may be performed simultaneously, sequentially, and/or in a different order than shown in FIG. 22. Flow diagram 2200 shall be described with reference to FIGS. 13, 14A-14C, 15-18, 19A-19C, and 23. However, flow diagram 2200 is not limited to those example aspects. Although flow diagram 2200 is shown in FIG. 22 as a stand-alone method, the aspects of this disclosure can be used with other processes, apparatuses, systems, and/or methods, such as, but not limited to, elements in FIGS. 13, 14A-14C, 15-18, 19A-19C, 20, 21, and 23, e.g., control diagram 1400A, therapy assessment 1400B, control diagram 1400C, therapy assessment 1430, one or more dosing regimen 1410, 1440, 1450, 1460, 1480, flow diagram 2000, flow diagram 2100, and/or computing device 2300. In some aspects, flow diagram 2200 can be implemented by software application 1390. In some aspects, flow diagram 2200 can be implemented by one or more models or algorithms run on one or more processors and/or computing devices based on one or more instructions stored in one or more memories.

In step 2202, as shown in the example of FIGS. 13-18, 19A-19C, and 23, glucose data of a user in a first time period can be received from an in vivo glucose monitoring device. In some aspects, glucose data can be received from OBU 1350 of therapy management system 1300 (e.g., via software application 1390). In some aspects, the first time period can include a morning window or fasting period.

In step 2204, as shown in the example of FIGS. 13-18, 19A-19C, and 23, a first minimum glucose metric can be determined for the first time period. In some aspects, software application 1390 of therapy management system 1300 can receive glucose data in the first time period and calculate the first minimum glucose metric. In some aspects, the first minimum glucose metric can include fasting glucose, DMG, MMG, average glucose for a predetermined time period, DMHAG, or a combination thereof.

In step 2206, as shown in the example of FIGS. 13-18, 19A-19C, and 23, a dose recommendation can be provided to the user based on the glucose data in the first time period. In some aspects, software application 1390 of therapy management system 1300 can calculate the dose recommendation based on glucose levels and/or one or more glucose metrics (e.g., minimum glucose metrics) of the glucose data in the first time period.

In step 2208, as shown in the example of FIGS. 13-18, 19A-19C, and 23, glucose data of the user in a second time period following the dose recommendation can be received from the in vivo glucose monitoring device. In some aspects, glucose data can be received from OBU 1350 of therapy management system 1300 (e.g., via software application 1390). In some aspects, the second time period can include a window after the dose recommendation is provided. In some aspects, the second time period can include a window after the user confirms administration of the dose recommendation.

In step 2210, as shown in the example of FIGS. 13-18, 19A-19C, and 23, a second minimum glucose metric can be determined for the second time period. In some aspects, software application 1390 of therapy management system 1300 can receive glucose data in the second time period and calculate the second minimum glucose metric. In some aspects, the second minimum glucose metric can include fasting glucose, DMG, MMG, average glucose for a predetermined time period, DMHAG, or a combination thereof. In some aspects, the first and second minimum glucose metrics can be the same type (e.g., both MMG, both DMHAG, etc.). In some aspects, the first and second minimum glucose metrics can be different (e.g., first minimum glucose can be MMG, second minimum glucose can be DMHAG, etc.).

In step 2212, as shown in the example of FIGS. 13-18, 19A-19C, and 23, non-adherence to the dose recommendation can be determined based on a comparison of the first and second minimum glucose metrics. In some aspects, the comparison can be based on whether the first and second minimum glucose metrics change by a predetermined amount (e.g., change in MMG is outside a range of a predicted change). In some aspects, the comparison can be based on whether the first and second minimum glucose metrics change in a correct direction. For example, for a dose recommendation that increases a dose amount, non-adherence can be determined if there is not a subsequent measured drop in glucose levels. In some aspects, software application 1390 of therapy management system 1300 can receive the glucose data for the first and second time periods, determine the first and second minimum glucose metrics, and compare the first and second minimum glucose metrics.

In step 2214, as shown in the example of FIGS. 13-18, 19A-19C, and 23, an indication of non-adherence can be outputted, for example, based on the comparison. In some aspects, software application 1390 of therapy management system 1300 can output an indication of non-adherence (e.g., via remote device 1310) to the user and/or a HCP (e.g., third party).

In some aspects, software application 1390 can include one or more models or algorithms (e.g., as described herein) run on one or more processors and/or computing devices based on one or more instructions stored in one or more memories. In some aspects, software application 1390 can be stored and/or executed on a phone (e.g., remote device 1310), a medication delivery device (e.g., first injection pen 1360, second injection pen 1370), a pen cap (e.g., first smart cap 1382, second smart cap 1384), on a cloud server, or a combination thereof. In some aspects, the one or more algorithms as described above (e.g., control diagram 1400A, therapy assessment 1400B, control diagram 1400C, therapy assessment 1430, flow diagram 2000, flow diagram 2100, etc.) for titration and/or dose recommendations can be stored and/or executed on a phone (e.g., remote device 1310), a medication delivery device (e.g., first injection pen 1360, second injection pen 1370), a pen cap (e.g., first smart cap 1382, second smart cap 1384), on a cloud server, or a combination thereof.

In some aspects, the one or more therapy regimen can include pre-mixed insulin comprising a combination of long-acting (LA) insulin and rapid-acting (RA) insulin. In some aspects, basal insulin therapy and/or prandial insulin therapy can utilize pre-mixed insulin. For example, third dosing regimen 1450 can utilize pre-mixed insulin with one meal for a single combined dose of LA (basal) insulin and RA (bolus) insulin.

In some aspects, software application 1390 can include one or more titration algorithms. In some aspects, software application 1390 can utilize multiple titration algorithms (e.g., general, personalized, optimized). In some aspects, software application 1390 can provide an option for a user to select a particular titration algorithm. In some aspects, software application 1390 can include a general titration algorithm based on American Diabetes Association (ADA) guidelines (e.g., fasting blood glucose levels of 80-130 mg/dL, starting dose of insulin of 10 units/day or 0.1-0.2 units/kg/day, insulin dose increments of 5-15% or 1-4 units, etc.). In some aspects, software application 1390 can include a personalized titration algorithm that is personalized to the user (e.g., control diagram 1400A, therapy assessment 1400B, control diagram 1400C, therapy assessment 1430, flow diagram 2000, flow diagram 2100, etc.), for example, based on one or more glucose metrics and/or one or more user metrics as described herein. In some aspects, software application 1390 can include an optimized titration algorithm that is based on Al and/or machine learning, for example, a constructed user profile can be generated using an Al module trained using analytics. In some aspects, the optimized titration algorithm can utilize simulated annealing, gradient descent, finite difference, interpolation, population models, regression, parameter adaptation, supervised machine learning, unsupervised machine learning, neural networks, classification models, clustering, vector quantization, stochastic gradient descent, implicit updates, leaky averaging, momentum methods, adaptive gradient (AdaGrad), backpropagation, root mean square propagation (RMSProp), adaptive moment estimation (Adam), or a combination thereof.

### Exemplary Computing Device

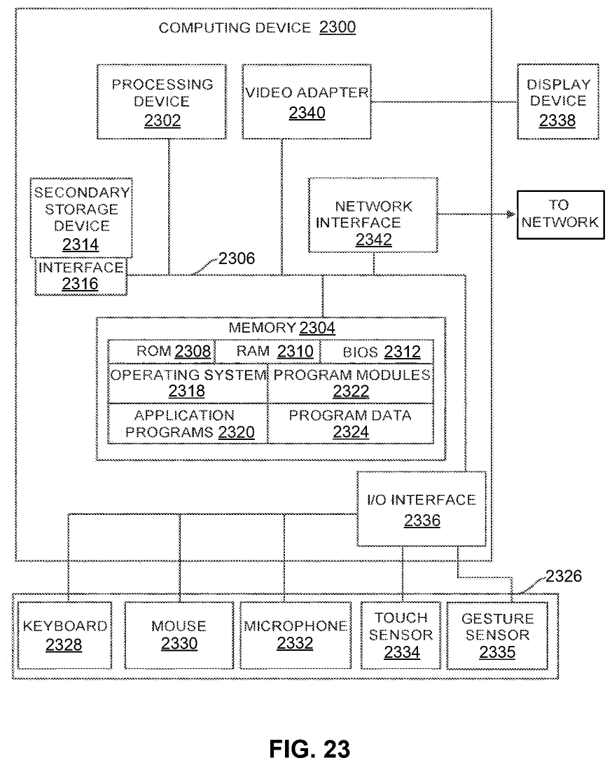

FIG. 23 illustrates computing device 2300, according to various exemplary aspects. Computing device 2300 can be configured to implement the operations described herein. Computing device 2300 can be further configured to execute the operating system, application programs, control diagrams, flow diagrams, and/or software modules (e.g., software engines) described herein. For example, computing device 2300 can be configured to implement control diagram 1400A shown in FIG. 14A. For example, computing device 2300 can be configured to implement flow diagram 2000 shown in FIG. 20. For example, computing device 2300 can be configured to implement flow diagram 2100 shown in FIG. 21. For example, computing device 2300 can be configured to implement flow diagram 2200 shown in FIG. 22. Although computing device 2300 is shown in FIG. 23 as a stand-alone apparatus and/or system, the aspects of this disclosure can be used with other apparatuses, systems, and/or methods, such as, but not limited to, elements in FIGS. 14A-18, 19A-19C, and 20-22, e.g., control diagram 1400A, therapy assessment 1430, one or more dosing regimen 1410, 1440, 1450, 1460, 1480, flow diagram 2000, flow diagram 2100, and/or flow diagram 2200.

The aspects of computing device 1200 shown in FIG. 12, for example, and the aspects of computing device 2300 shown in FIG. 23 may be similar. Similar reference numbers are used to indicate features of the aspects of computing device 1200 shown in FIG. 12 and the similar features of the aspects of computing device 2300 shown in FIG. 23. Computing device 1200 can implement the operations described herein and execute the operating system, application programs, control diagrams, flow diagrams, and/or software modules (e.g., software engines) described herein.

As shown in FIG. 23, computing device 2300 can include processing device 2302 (e.g., one or more processors, one or more microprocessors, microcontroller), system memory 2304 (e.g., one or more memories), system bus 2306, read-only memory (ROM) 2308, random-access memory (RAM) 2310, basic input/output system (BIOS) 2312, secondary storage device 2314 (e.g., a hard drive), secondary storage interface 2316, operating system (OS) 2318, application programs (apps) 2320, program modules 2322 (e.g., software engines, algorithms), program data 2324, input devices 2326 (e.g., keyboard 2328, mouse 2330, microphone 2332, touch sensor 2334, gesture sensor 2335), input/output (I/O) interface 2336, display device 2338, video adapter 2340, and network interface 2342.

In some aspects, computing device 2300 can include at least one processing device 2302 (e.g., a processor), such as a central processing unit (CPU). A variety of processing devices are available from a variety of manufacturers, for example, Intel or Advanced Micro Devices. In this example, computing device 2300 also includes system memory 2304 and system bus 2306 that couples various system components including system memory 2304 to processing device 2302. System bus 2306 can be any number of types of bus structures that can be used, including, but not limited to, a memory bus, or memory controller; a peripheral bus; or a local bus using any of a variety of bus architectures.

Examples of computing devices that can be implemented using computing device 2300 include a desktop computer, a laptop computer, a tablet computer, a mobile computing device (such as a smart phone, a touchpad mobile digital device, or other mobile devices), or other devices configured to process digital instructions.

System memory 2304 includes ROM 2308 and RAM 2310. A BIOS 2312 containing basic routines that act to transfer information within computing device 2300, such as during start up, can be stored in ROM 2308.

In some aspects, computing device 2300 can include secondary storage device 2314, such as a hard disk drive, for storing digital data. Secondary storage device 2314 can be connected to system bus 2306 by secondary storage interface 2316. Secondary storage device 2314 and its associated computer readable media can provide nonvolatile and non-transitory storage of computer readable instructions (e.g., including application programs and program modules), data structures, and other data for computing device 2300.

Although the example environment described herein employs a hard disk drive as a secondary storage device, other types of computer readable storage media can be used in other aspects. Examples of these other types of computer readable storage media can include magnetic cassettes, flash memory cards, digital video disks, Bernoulli cartridges, compact disc read only memories, digital versatile disk read only memories, random access memories, or read only memories. Some aspects can include non-transitory media. For example, a computer program product can be tangibly embodied in a non-transitory storage medium. Additionally, such computer readable storage media can include local storage or cloud-based storage.

A number of program modules can be stored in secondary storage device 2314 and/or system memory 2304, including OS 2318, one or more application programs 2320, other program modules 2322 (such as the software applications and software engines described herein), and program data 2324. Computing device 2300 can utilize any suitable operating system, such as Microsoft Windows™, Google Chrome™ OS, Apple OS, Unix, or Linux and variants and any other operating system suitable for a computing device. Other examples can include Microsoft, Google, or Apple operating systems, or any other suitable operating system used in tablet computing devices.

In some aspects, a user can provide one or more inputs to computing device 2300 through one or more input devices 2326. Examples of input devices 2326 can include keyboard 2328, mouse 2330, microphone 2332 (e.g., for voice and/or other audio input), touch sensor 2334 (e.g., a touchpad and/or touch sensitive display), and gesture sensor 2335 (e.g., for gestural input). In some aspects, input devices 2326 can provide detection based on presence, proximity, and/or motion. In some aspects, a user can walk into their home, and this can trigger an input into processing device 2302. For example, input devices 2326 may then facilitate an automated experience for the user. Other aspects can include other input devices 2326. Input devices 2326 can be connected to processing device 2302 through I/O interface 2336 that can be coupled to system bus 2306. Input devices 2326 can be connected by any number of I/O interfaces, such as a parallel port, serial port, game port, and/or a universal serial bus. In some aspects, input devices 2326 can be in wireless communication with I/O interface 2336, for example, including infrared, BLUETOOTH® wireless technology, 802.11a/b/g/n, cellular, ultra-wideband (UWB), ZigBee, or other radio frequency (RF) communication systems.

In this example aspect, display device 2338, such as a monitor, liquid crystal display device, light-emitting diode display device, projector, or touch sensitive display device, can also be connected to system bus 2306 via an interface, such as a video adapter 2340. In some aspects, in addition to display device 2338, computing device 2300 can include various other peripheral devices (not shown), such as speakers or a printer.

Computing device 2300 can be connected to one or more networks through network interface 2342. Network interface 2342 can provide for wired and/or wireless communication. In some aspects, network interface 2342 can include one or more antennas for transmitting and/or receiving wireless signals. In some aspects, when used in a local area networking environment or a wide area networking environment (e.g., such as the Internet), network interface 2342 can include an Ethernet interface. Other possible aspects can use other communication devices. For example, some aspects of computing device 2300 can include a modem for communicating across the network.

Computing device 2300 can include at least some form of computer readable media. Computer readable media can include any available media that can be accessed by computing device 2300. For example, computer readable media can include computer readable storage media and computer readable communication media.

Computer readable storage media can include volatile and nonvolatile, removable and non-removable media implemented in any device configured to store information, such as computer readable instructions, data structures, program modules, or other data. Computer readable storage media can include, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by computing device 2300.

Computer readable communication media can include computer readable instructions, data structures, program modules, or other data in a modulated data signal, such as a carrier wave or other transport mechanism and can include any information delivery media. The term “modulated data signal” refers to a signal that has one or more of its characteristics set or changed in such a manner as to encode information in the signal. For example, computer readable communication media can include wired media, such as a wired network or direct-wired connection, and wireless media such as acoustic, radio frequency, infrared, and other wireless media. Combinations of any of the above are also included within the scope of computer readable media.

Computing device 2300 illustrated in FIG. 23 is also an example of programmable electronics, which can include one or more such computing devices, and when multiple computing devices are included, such computing devices can be coupled together with a suitable data communication network so as to collectively perform the various functions, methods, or operations disclosed herein.

Various aspects of the disclosure can be implemented on a computing device by software, firmware, hardware, or a combination thereof. FIG. 23 illustrates an exemplary computing device 2300 in which the contemplated aspects, or portions thereof, can be implemented as computer-readable code. For example, the methods illustrated by control diagrams described herein can be implemented in computing device 2300. Various aspects are described in terms of this exemplary computing device 2300. After reading the above description, it will become apparent to a person skilled in the relevant art how to implement the aspects using other computer systems and/or computer architectures.

It is to be understood that the phraseology or terminology herein is for the purpose of description and not of limitation, such that the terminology or phraseology of the present specification is to be interpreted by those skilled in relevant art(s) in light of the teachings herein.

The above examples are illustrative, but not limiting, of the aspects of this disclosure. Other suitable modifications and adaptations of the variety of conditions and parameters normally encountered in the field, and which would be apparent to those skilled in the relevant art(s), are within the spirit and scope of the disclosure.

While specific aspects have been described above, it will be appreciated that the aspects may be practiced otherwise than as described. The description is not intended to limit the scope of the claims.

It is to be appreciated that the Detailed Description section, and not the Summary and Abstract sections, is intended to be used to interpret the claims. The Summary and Abstract sections may set forth one or more but not all exemplary aspects as contemplated by the inventor(s), and thus, are not intended to limit the aspects and the appended claims in any way.

The aspects have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the aspects that others can, by applying knowledge within the skill of the art, readily modify and/or adapt for various applications such specific aspects, without undue experimentation, without departing from the general concept of the aspects. Therefore, such adaptations and modifications are intended to be within the meaning and range of equivalents of the disclosed aspects, based on the teaching and guidance presented herein.

The breadth and scope of the aspects should not be limited by any of the above-described exemplary aspects, but should be defined only in accordance with the following claims and their equivalents.

The present invention may also be described in accordance with the following clauses:
Clause 1. A non-transitory computer-readable storage medium, the non-transitory computer-readable storage medium having stored thereon instructions that when executed by a processor causes the processor to perform operations of a management process for insulin therapy, the operations comprising:
performing a survey of a person with diabetes (PWD) and receiving survey information in response to the survey;
performing a high-touch interaction phase with regard to the PWD, the high-touch interaction phase including repeated adjustments of the insulin therapy based on collected data and a publicly available guideline regarding the insulin therapy, the high-touch interaction phase establishing, for the PWD, at least a basal dose setting, a meal dose setting, and a correction dose setting; and
performing, after termination of the high-touch interaction phase, an ongoing phase with regard to the PWD, the ongoing phase including collecting performance metrics for the PWD, applying reinforcement learning to the performance metrics, and automatically performing an action with regard to the PWD based on the reinforcement learning.
Clause 2. The non-transitory computer-readable storage medium of clause 1, wherein the high-touch interaction phase is performed for a predetermined length of time.
Clause 3. The non-transitory computer-readable storage medium storage medium of clause 1, wherein the high-touch interaction phase has a goal-based duration.
Clause 4. The non-transitory computer-readable storage medium of clause 3, wherein the goal-based duration depends on a determination that a blood glucose level of the PWD is within a target range, and that the PWD is taking insulin as specified in the insulin therapy.
Clause 5. The non-transitory computer-readable storage medium of clause 1, the operations further comprising evaluating each of multiple insights and determining whether the insight is triggered in view of at least the performance metrics and the collected data, wherein the action is selected based on at least one of the insights being triggered.
Clause 6. The non-transitory computer-readable storage medium of clause 5, wherein the insights are evaluated according to priority with regard to the PWD.
Clause 7. The non-transitory computer-readable of clause 6, wherein the insights being evaluated are selected from a set of insights based on having highest priority.
Clause 8. The non-transitory computer-readable storage medium of clause 5, wherein evaluating at least one of the insights comprises a multimodal evaluation.
Clause 9. The non-transitory computer-readable storage medium of clause 1, the operations further comprising performing observation with regard to a state of the PWD after automatically performing the action.
Clause 10. The non-transitory computer-readable storage medium of clause 9, wherein the reinforcement learning comprises providing positive feedback or negative feedback to a selection of the action.
Clause 11. The non-transitory computer-readable storage medium of clause 10, the operations further comprising performing a cooldown after termination of the observation.
Clause 12. The non-transitory computer-readable storage medium of clause 11, the operations further comprising evaluating each of multiple insights and determining whether the insight is triggered in view of at least the performance metrics and the collected data, wherein the action is selected based on at least one of the insights being triggered.
Clause 13. The non-transitory computer-readable storage medium of clause 12, the operations further comprising checking, before selecting the action, whether the cooldown involves the action.
Clause 14. The non-transitory computer-readable storage medium of clause 13, wherein if the cooldown involves the action, the operations instead evaluate a next insight of the multiple insights.
Clause 15. The non-transitory computer-readable storage medium of clause 9, the operations further comprising evaluating each of multiple insights and determining whether the insight is triggered in view of at least the performance metrics and the collected data, wherein the action is selected based on at least one of the insights being triggered.
Clause 16. The non-transitory computer-readable storage medium of clause 15, the operations further comprising checking, before selecting the action, whether the observation involves the insight.
Clause 17. The non-transitory computer-readable storage medium of clause 14, wherein if the observation involves the insight, the operations evaluate an improvement criterion regarding the insight in view of at least the performance metrics and the collected data.
Clause 18. The non-transitory computer-readable storage medium of clause 17, wherein evaluating the improvement criterion comprises a multimodal evaluation.
Clause 19. The non-transitory computer-readable storage medium of clause 14, wherein for any of the insights not being triggered in the evaluation, the operations further comprise determining whether that insight has not been triggered for at least a predetermined time, and performing a streak action in view of the determination.
Clause 20. The non-transitory computer-readable storage medium of clause 1, wherein automatically performing the action comprises selecting one or more goals for the PWD, and presenting the one or more goals to the PWD.
Clause 21. The non-transitory computer-readable storage medium of clause 20, the operations further comprising receiving an opt-in input or an opt-out input by the PWD.
Clause 22. A method of therapy escalation for patients with diabetes, the method comprising:
receiving glucose data of a user from an in vivo glucose monitoring device;
receiving first therapy information of a first therapy, wherein the first therapy comprises basal insulin;
calculating one or more glucose metrics based on the received glucose data;
titrating a dose of the basal insulin based on the one or more glucose metrics; and
determining overbasalization based on one or more of the glucose data and the first therapy information.
Clause 23. The method of clause 22, further comprising outputting a recommendation to add a second therapy when overbasalization is determined.
Clause 24. The method of clause 22 or clause 23, wherein the one or more glucose metrics comprises average glucose, median glucose, glucose time-in-range (TIR), glucose time below range (TBR), glucose time above range (TAR), glucose time very low (TVL), glucose management indicator (GMI), minimum morning glucose (MMG), minimum post-dose glucose (MPDG), post-prandial glucose (PPG), bedtime to AM glucose (BeAM), or a combination thereof.
Clause 25. The method of any one of clauses 22-24, wherein the first therapy information is manually entered via an input of a computing device.
Clause 26. The method of any one of clauses 22-25, wherein the first therapy information is collected by a medication delivery device and is communicated to a computing device.
Clause 27. The method of any one of clauses 22-26, wherein overbasalization is determined based on a ratio of total daily dose (TDD) of insulin to a body weight of the user.
Clause 28. The method of any one of clauses 22-27, wherein overbasalization is determined based on glucose variability.
Clause 29. The method of any one of clauses 22-28, wherein overbasalization is determined based on a hypoglycemia metric.
Clause 30. The method of any one of clauses 22-29, wherein overbasalization is determined based on a change in glucose level in a meal period or an overnight period.
Clause 31. The method of any one of clauses 22-30, further comprising decreasing the basal insulin dose when the second therapy is added.
Clause 32. The method of any one of clauses 22-31, wherein the second therapy comprises a glucagon-like peptide-1 (GLP-1) receptor agonist.
Clause 33. The method of clause 32, further comprising titrating a dose of the GLP-1 receptor agonist, wherein titrating the dose comprises:
receiving a user input on side-effects of the second therapy, and
recommending an increased dose when the user input is associated with no side-effects.
Clause 34. The method of clause 32, further comprising titrating a dose of the GLP-1 receptor agonist, wherein titrating the dose comprises:
receiving a body weight measurement of the user, and
recommending an increased dose when the body weight measurement is above a target body weight.
Clause 35. The method of any one of clauses 22-34, wherein the one or more glucose metrics comprises a post-meal glucose rise for each of a plurality of meals.
Clause 36. The method of clause 35, further comprising recommending initiating a prandial insulin dose for a first meal when the post-meal glucose rise for one meal exceeds a threshold.
Clause 37. The method of clause 35, further comprising recommending initiating a prandial insulin dose for a plurality of meals when the post-meal glucose rise for each of the plurality of meals exceeds a threshold.
Clause 38. A system of therapy escalation for patients with diabetes, the system comprising:
an in vivo glucose monitoring device configured to measure glucose data of a user;
a remote device in communication with the in vivo glucose monitoring device, wherein the remote device is configured to receive or retrieve glucose data from the in vivo glucose monitoring device;
a medication delivery device in communication with the in vivo glucose monitoring device and the remote device, wherein the medication delivery device is configured to administer one or more dosing regimen; and
a processor in communication with the analyte measurement system, the remote device, and the medication delivery device, wherein the processor is coupled to a memory storing instructions that when executed cause the processor to perform operations comprising:
receiving glucose data of the user from the in vivo glucose monitoring device;
receiving first therapy information of a first therapy from the remote device, the medication delivery device, or both, wherein the first therapy comprises basal insulin;
calculating one or more glucose metrics based on the received glucose data;
titrating a dose of the basal insulin based on the one or more glucose metrics; determining overbasalization based on one or more of the glucose data and the first therapy information; and
outputting a recommendation to the remote device to add a second therapy when overbasalization is determined.
Clause 39. The system of clause 38, wherein the operations further comprise outputting a recommendation to the remote device to add a second therapy when overbasalization is determined.
Clause 40. The system of clause 38 or clause 39, wherein the one or more glucose metrics comprises average glucose, median glucose, glucose TIR, glucose TBR, glucose TAR, glucose TVL, GMI, MMG, MPDG, PPG, BeAM, a post-meal glucose rise for each of a plurality of meals, or a combination thereof.
Clause 41. The system of any one of clauses 38-40, wherein overbasalization is determined based on a ratio of TDD of insulin to a body weight of the user.
Clause 42. The system of any one of clauses 38-41, wherein overbasalization is determined based on glucose variability, a hypoglycemia metric, a change in glucose level in a meal period or an overnight period, or a combination thereof.
Clause 43. The system of any one of clauses 38-42, wherein the second therapy comprises a GLP-1 receptor agonist, a prandial insulin dose for a first meal, a prandial insulin dose for a plurality of meals, or a combination thereof.
Clause 44. A method of therapy escalation for patients with diabetes to detect non-adherence to a basal insulin recommendation, the method comprising:
receiving glucose data of a user from an in vivo glucose monitoring device;
recommending a first dose of basal insulin to the user;
calculating one or more glucose metrics based on the received glucose data, wherein the one or more glucose metrics comprises a minimum glucose metric;
titrating a recommended dose of the basal insulin based on the one or more glucose metrics;
recommending a second dose of basal insulin to the user, wherein the second dose is different from the first dose due to titrating the recommended dose;
calculating a change in the minimum glucose metric from a first time period prior to recommending the second dose to a second time period after recommending the second dose;
determining whether the change in the minimum glucose metric is outside a predetermined metric; and
outputting, if the change in the minimum glucose metric is outside the predetermined metric, an indication that titration of the basal insulin will stop.
Clause 45. The method of clause 44, wherein the minimum glucose metric comprises daily minimum hourly average glucose (DMHAG).
Clause 46. The method of clause 44 or clause 45, wherein the predetermined metric comprises the change in the minimum glucose metric being less than a predetermined percentage of an expected change of the minimum glucose metric.
Clause 47. The method of clause 46, wherein the expected change of the minimum glucose metric is based on one or more user metrics.
Clause 48. The method of clause 47, wherein the one or more user metrics comprises insulin sensitivity factor (ISF).
Clause 49. The method of clause 46, wherein the expected change of the minimum glucose metric is based on historical glucose data of the user.
Clause 50. The method of clause 46, wherein the expected change of the minimum glucose metric is proportional to a change between the first dose and the second dose.
Clause 51. The method of any one of clauses 44-50, wherein the predetermined metric comprises the change in the minimum glucose metric is less than a predetermined change in the glucose level.
Clause 52. The method of any one of clauses 44-51, wherein the predetermined metric comprises determining a change in the minimum glucose metric based on a statistical method.
Clause 53. The method of any one of clauses 44-52, further comprising prompting the user to confirm administration of the second dose.
Clause 54. The method of clause 53, further comprising, if the user confirms administration of the second dose, resuming titration.
Clause 55. The method of clause 53, further comprising recommending, if the user does not confirm administration of the second dose, a third dose based on titrating the second dose.
Clause 56. The method of any one of clauses 44-55, further comprising recommending limiting a change in dose amount of basal insulin if there is no change in the minimum glucose metric after titrating the recommended dose for a predetermined time period.
Clause 57. The method of clause 56, wherein the predetermined time period comprises at least 3 days.
Clause 58. The method of any one of clauses 44-57, further comprising stopping upward titration of basal insulin if the minimum glucose metric does not decrease after titrating the recommended dose for a predetermined time period.
Clause 59. The method of any one of clauses 44-58, further comprising stopping downward titration of basal insulin if the minimum glucose metric does not increase after titrating the recommended dose for a predetermined time period.
Clause 60. The method of any one of clauses 44-59, further comprising activating a blinded mode such that glucose data is not displayed to the user.
Clause 61. The method of any one of clauses 44-60, further comprising initiating, if the change in the minimum glucose metric is outside the predetermined metric, a counter configured to monitor a number of titration cycles; and recommending stopping titration of basal insulin if the change in the minimum glucose metric remains outside the predetermined metric after a predetermined number of titration cycles with increasing or decreasing doses of basal insulin.
Clause 62. The method of any one of clauses 44-61, further comprising outputting a prompt to the user to have the user seek guidance from a health care professional.
Clause 63. The method of any one of clauses 44-62, further comprising outputting a quiz to the user to verify the user is following the recommended dose
Clause 64. A method of detecting non-adherence to a dose recommendation, the method comprising:
receiving glucose data of a user in a first time period from an in vivo glucose monitoring device;
determining a first minimum glucose metric for the first time period;
providing a dose recommendation to the user based on the glucose data in the first time period;
receiving glucose data of the user in a second time period following the dose recommendation;
determining a second minimum glucose metric for the second time period;
determining non-adherence to the dose recommendation based on a comparison of the first and second minimum glucose metrics; and
outputting an indication of non-adherence.
Clause 65. The method of clause 64, wherein the first and second minimum glucose metrics comprise daily minimum hourly average glucose (DMHAG).
Clause 66. The method of clause 64 or clause 65, wherein the comparison comprises a change between the first and second minimum glucose metrics.
Clause 67. The method of clause 66, wherein determining non-adherence comprises comparing the change between the first and second minimum glucose metrics to a predetermined percentage change threshold.
Clause 68. The method of clause 66, wherein determining non-adherence comprises comparing the change between the first and second minimum glucose metrics to a predetermined change in the glucose level.
Clause 69. The method of any one of clauses 64-68, wherein the comparison comprises a direction of the first and second minimum glucose metrics.
Clause 70. The method of clause 69, wherein the direction is inversely proportional to the dose recommendation.

1-21. (canceled)

22. A method of therapy escalation for patients with diabetes, the method comprising:
receiving glucose data of a user from an in vivo glucose monitoring device;
receiving first therapy information of a first therapy, wherein the first therapy comprises basal insulin;
calculating one or more glucose metrics based on the received glucose data;
titrating a dose of the basal insulin based on the one or more glucose metrics; and
determining overbasalization based on one or more of the glucose data and the first therapy information.

23. The method of claim 22, further comprising outputting a recommendation to add a second therapy when overbasalization is determined.

24. The method of claim 22, wherein the one or more glucose metrics comprises average glucose, median glucose, glucose time-in-range (TIR), glucose time below range (TBR), glucose time above range (TAR), glucose time very low (TVL), glucose management indicator (GMI), minimum morning glucose (MMG), minimum post-dose glucose (MPDG), post-prandial glucose (PPG), bedtime to AM glucose (BeAM), or a combination thereof.

25. The method of claim 22, wherein the first therapy information is manually entered via an input of a computing device.

26. The method of claim 22, wherein the first therapy information is collected by a medication delivery device and is communicated to a computing device.

27. The method of claim 22, wherein overbasalization is determined based on a ratio of total daily dose (TDD) of insulin to a body weight of the user.

28. The method of claim 22, wherein overbasalization is determined based on glucose variability.

29. The method of claim 22, wherein overbasalization is determined based on a hypoglycemia metric.

30. The method of claim 22, wherein overbasalization is determined based on a change in glucose level in a meal period or an overnight period.

31. The method of claim 22, further comprising decreasing the basal insulin dose when the second therapy is added.

32. The method of claim 22, wherein the second therapy comprises a glucagon-like peptide-1 (GLP-1) receptor agonist.

33. The method of claim 32, further comprising titrating a dose of the GLP-1 receptor agonist, wherein titrating the dose comprises:
receiving a user input on side-effects of the second therapy, and
recommending an increased dose when the user input is associated with no side-effects.

34. The method of claim 32, further comprising titrating a dose of the GLP-1 receptor agonist, wherein titrating the dose comprises:
receiving a body weight measurement of the user, and
recommending an increased dose when the body weight measurement is above a target body weight.

35. The method of claim 22, wherein the one or more glucose metrics comprises a post-meal glucose rise for each of a plurality of meals.

36. The method of claim 35, further comprising recommending initiating a prandial insulin dose for a first meal when the post-meal glucose rise for one meal exceeds a threshold.

37. The method of claim 35, further comprising recommending initiating a prandial insulin dose for a plurality of meals when the post-meal glucose rise for each of the plurality of meals exceeds a threshold.

38. A system of therapy escalation for patients with diabetes, the system comprising:
an in vivo glucose monitoring device configured to measure glucose data of a user;
a remote device in communication with the in vivo glucose monitoring device, wherein the remote device is configured to receive or retrieve glucose data from the in vivo glucose monitoring device;
a medication delivery device in communication with the in vivo glucose monitoring device and the remote device, wherein the medication delivery device is configured to administer one or more dosing regimen; and
a processor in communication with the analyte measurement system, the remote device, and the medication delivery device, wherein the processor is coupled to a memory storing instructions that when executed cause the processor to perform operations comprising:
receiving glucose data of the user from the in vivo glucose monitoring device;
receiving first therapy information of a first therapy from the remote device, the medication delivery device, or both, wherein the first therapy comprises basal insulin;
calculating one or more glucose metrics based on the received glucose data;
titrating a dose of the basal insulin based on the one or more glucose metrics; and
determining overbasalization based on one or more of the glucose data and the first therapy information.

39. The system of claim 38, wherein the operations further comprise outputting a recommendation to the remote device to add a second therapy when overbasalization is determined.

40. The system of claim 38, wherein the one or more glucose metrics comprises average glucose, median glucose, glucose TIR, glucose TBR, glucose TAR, glucose TVL, GMI, MMG, MPDG, PPG, BeAM, a post-meal glucose rise for each of a plurality of meals, or a combination thereof.

41. The system of claim 38, wherein overbasalization is determined based on a ratio of TDD of insulin to a body weight of the user.

42-70. (canceled)
